SEQUENCE LISTING (6 sequences)

SEQ ID NO: 1 - DNA, 21 bp, Artificial Sequence
  Description of Artificial Sequence Synthetic primer
gactttgctttccttggtcag

SEQ ID NO: 2 - DNA, 24 bp, Artificial Sequence
  Description of Artificial Sequence Synthetic primer
ggcttatatccaacacttcgtggg

SEQ ID NO: 3 - DNA, 26 bp, Artificial Sequence
  Description of Artificial Sequence Synthetic probe
atggtcaaggtcgcaagcttgctggt

SEQ ID NO: 4 - DNA, 18 bp, Artificial Sequence
  Description of Artificial Sequence Synthetic primer
tgtgcagaaggatggagt

SEQ ID NO: 5 - DNA, 20 bp, Artificial Sequence
  Description of Artificial Sequence Synthetic primer
ctggtgcttctctcaggata

SEQ ID NO: 6 - DNA, 25 bp, Artificial Sequence
  Description of Artificial Sequence Synthetic probe
tggaatatgccctgcgtaaactgga

(19) United States
(12) Patent Application Publication
(10) Pub. No.: US 2012/0095200 A1
(43) Pub. Date: Apr. 19, 2012

(54) COMPOSITIONS AND METHODS FOR THE SPECIFIC INHIBITION OF GENE EXPRESSION BY NUCLEIC ACID CONTAINING A DICER SUBSTRATE AND A RECEPTOR BINDING REGION

(75) Inventor: Bob Dale Brown, Millington, NJ (US)

(73) Assignee: Dicerna Pharmaceuticals, Inc., Watertown, MA (US)

(21) Appl. No.: 13/298,928

(22) Filed: Nov. 17, 2011

Related U.S. Application Data
(63) Continuation-in-part of application No. PCT/US10/37586, filed on Jun. 7, 2010.
(60) Provisional application No. 61/184,735, filed on Jun. 5, 2009.

(51) Int. Cl. C07H 21/02; C12P 19/34

(52) U.S. Cl. 536/23.1; 435/91.3

(57) ABSTRACT

The invention features compositions and methods that are useful for reducing the expression or activity of a specified gene in a eukaryotic cell, involving contacting a cell with an isolated nucleic acid containing a Dicer substrate and a receptor binding region in an amount effective to reduce expression of a target gene in a cell.

### CROSS-REFERENCE TO RELATED APPLICATIONS

The present application is a Continuation-in-Part of international application No. PCT/US2010/037586, filed Jun. 7, 2010, designating the United States, which claims priority under 35 U.S.C. §119(e) to U.S. provisional patent application No. 61/184,735, filed Jun. 5, 2009, the entire teachings of which are incorporated herein by reference.

### BACKGROUND OF THE INVENTION

Double-stranded RNA (dsRNA) molecules possessing strand lengths of 25 to 35 nucleotides have been described as effective inhibitors of target gene expression in mammalian cells (Rossi et al., U.S. Patent Application Nos. 2005/0244858 and US 2005/0277610). dsRNA molecules of such length are believed to be processed by the Dicer enzyme of the RNA interference (RNAi) pathway, leading such molecules to be termed “Dicer substrate siRNA” (“DsiRNA”) molecules. Certain modified structures of DsiRNA molecules were previously described (Rossi et al., U.S. Patent Application No. 2007/0265220). While robust, sequence-specific target gene silencing efficacy has been identified for 25-35 nucleotide length dsRNA molecules, a need exists for improved design of such molecules, including design of DsiRNA molecules possessing enhanced in vitro and in vivo efficacy.

Nucleic acid molecules that bind receptors can be isolated by in vitro selection methods (e.g., systematic evolution of ligands by exponential enrichment; “SELEX”). For example, SELEX has been used to identify nucleic acid aptamers that adopt conformations which allow them to bind molecules other than nucleic acids, such as polypeptides, specifically and with high affinity via non-Watson-Crick interactions.

### BRIEF SUMMARY OF THE INVENTION

In one aspect, the invention provides an isolated nucleic acid molecule containing a polynucleotide strand having a 5′ terminus and a 3′ terminus that is 53-142 nucleotides in length, the 5′ terminus and the 3′ terminus forming a double-stranded region of at least 21-25 base pairs, where the double-stranded region contains at least 19 nucleotides complementary to a target RNA, where the nucleic acid molecule selectively binds a receptor with an affinity of at least 100 μm, where Dicer cleavage of the nucleic acid molecule in the double-stranded region reduces target gene expression in a mammalian cell, and where Dicer cleavage of the nucleic acid molecule in the double-stranded region reduces the ability of the isolated nucleic acid to bind selectively to the receptor.

In another aspect, the invention provides an isolated nucleic acid molecule containing a first polynucleotide strand having a 5′ terminus and a 3′ terminus that is 33-121 nucleotides in length and a second polynucleotide strand having a 5′ terminus and a 3′ terminus that is 33-121 nucleotides in length, the 5′ terminus of the first polynucleotide strand and the 3′ terminus of the second polynucleotide strand forming a double-stranded region of at least 21-25 base pairs, where the double-stranded region comprises at least 19 nucleotides complementary to a target RNA, where the nucleic acid molecule selectively binds a receptor with an affinity of at least 100 μm, where Dicer cleavage of the nucleic acid molecule in the double-stranded region reduces target gene expression in a mammalian cell, and where Dicer cleavage of the nucleic acid molecule in the double-stranded region reduces the ability of the isolated nucleic acid to bind selectively to the receptor.

In one aspect, the invention provides a method of making a nucleic acid molecule that selectively binds a receptor and is a Dicer substrate, involving providing a nucleic acid molecule containing a single polynucleotide strand having a 5′ terminus and a 3′ terminus that is 53-142 nucleotides in length, the 5′ terminus and the 3′ terminus forming a double-stranded region of at least 21-25 base pairs, where the double-stranded region contains at least 19 nucleotides complementary to a target RNA; contacting the nucleic acid molecule with a receptor; isolating the nucleic acid molecule bound to the receptor; and contacting the isolated nucleic acid molecule with Dicer enzyme, where Dicer cleavage of the nucleic acid molecule in the double-stranded region reduces the ability of the aptamer to bind selectively to the receptor, thereby making a nucleic acid molecule that selectively binds a receptor and is a Dicer substrate.

In another aspect, the invention provides a method of making a nucleic acid molecule that selectively binds a receptor and is a Dicer substrate, involving providing a nucleic acid molecule containing a first polynucleotide strand having a 5′ terminus and a 3′ terminus that is 33-121 nucleotides in length and a second polynucleotide strand having a 5′ terminus and a 3′ terminus that is 33-121 nucleotides in length, the 5′ terminus of the first polynucleotide strand and the 3′ terminus of the second polynucleotide strand forming a double-stranded region of at least 21-25 base pairs, where the double-stranded region contains at least 19 nucleotides complementary to a target RNA; contacting the nucleic acid molecule with a receptor; isolating the nucleic acid molecule bound to the receptor; and contacting the isolated nucleic acid molecule with Dicer enzyme, where Dicer cleavage of the nucleic acid molecule in the double-stranded region reduces the ability of the aptamer to bind selectively to the receptor, thereby making a nucleic acid molecule that selectively binds a receptor and is a Dicer substrate.

In yet another aspect, the invention provides a method of making a nucleic acid molecule that selectively binds a receptor and is a Dicer substrate, involving providing a nucleic acid molecule containing (a) an aptamer containing a single polynucleotide strand having a 5′ terminus and a 3′ terminus that is 12-100 nucleotides in length, and (b) a double-stranded RNA (dsRNA) containing a first strand that is 25-30 nucleotides in length and a second strand that is 25-34 nucleotides in length, where the 3′ terminus of the first strand is covalently attached to the 5′ terminus of the aptamer and the 5′ end of the second strand is covalently attached to the 3′ terminus of the aptamer; contacting the nucleic acid molecule with a receptor; isolating the nucleic acid molecule bound to the receptor; and contacting the isolated nucleic acid molecule with Dicer enzyme, where Dicer cleavage of the dsRNA reduces the ability of the aptamer to bind selectively to the receptor, thereby making a nucleic acid molecule that selectively binds a receptor and is a Dicer substrate.

In still another aspect, the invention provides a method of making a nucleic acid molecule that selectively binds a receptor and is a Dicer substrate, involving providing a nucleic acid molecule containing (a) an aptamer containing a first polynucleotide strand having a 5′ terminus and a 3′ terminus that is 12-100 nucleotides in length and a second polynucleotide strand having a 5′ terminus and a 3′ terminus that is 12-100 nucleotides in length, and (b) a double-stranded RNA (dsRNA) containing a first strand that is 25-30 nucleotides in length and a second strand that is 25-34 nucleotides in length, where the 3′ terminus of the first strand of the dsRNA is covalently attached to the 5′ terminus of the first strand of the aptamer and the 5′ end of the second strand of the dsRNA is covalently attached to the 3′ terminus of the aptamer; contacting the nucleic acid molecule with a receptor; isolating the nucleic acid molecule bound to the receptor; and contacting the nucleic acid molecule with Dicer enzyme, where Dicer cleavage of the dsRNA reduces the ability of the aptamer to bind selectively to the receptor, thereby making a nucleic acid molecule that selectively binds a receptor and is a Dicer substrate.

In an another aspect, the invention provides an isolated nucleic acid molecule made by a method of any of the above aspects or any aspect delineated herein.

In an another aspect, the invention provides compositions and pharmaceutical compositions containing an isolated nucleic acid molecule of any of the above aspects or any aspect delineated herein

In various embodiments of any of the above aspects or any aspect delineated herein, the 5′ terminus and the 3′ terminus form a blunt end. In various embodiments of any of the above aspects or any aspect delineated herein, the 5′ terminus of the first polynucleotide strand and the 3′ terminus of the second polynucleotide strand form a blunt end. In various embodiments of any of the above aspects or any aspect delineated herein, the 5′ terminus and the 3′ terminus form a 1-4 nucleotide 3′ overhang. In specific embodiments, the nucleotides of the 3′ overhang contain a modified nucleotide. In specific embodiments, the 3′ overhang is two nucleotides in length and where the modified nucleotide of the 3′ overhang is a 2′-O-methyl modified ribonucleotide. In various embodiments of any of the above aspects or any aspect delineated herein, the 5′ terminus of the first polynucleotide strand and the 3′ terminus of the second polynucleotide strand form a 1-4 nucleotide 3′ overhang. In various embodiments of any of the above aspects or any aspect delineated herein, the first two nucleotides of the 5′ terminus and the ultimate and penultimate nucleotides of the 3′ terminus form one or two mismatched base pairs. In various embodiments of any of the above aspects or any aspect delineated herein, the 5′ terminus of the first polynucleotide strand and the 3′ terminus of the second polynucleotide strand form one or two mismatched base pairs.

In various embodiments of any of the above aspects or any aspect delineated herein, the receptor binding affinity is 1-100 μm. In various embodiments of any of the above aspects or any aspect delineated herein, the receptor binding affinity is 1-100 nm. In various embodiments of any of the above aspects or any aspect delineated herein, the receptor binding affinity is 1-100 μm.

In various embodiments of any of the above aspects or any aspect delineated herein, the isolated nucleic acid contains an internally base-paired region and a single-stranded region forming a hairpin, the internally base-paired region containing 4 consecutive base pairs and the single-stranded region containing 5 consecutive non-base paired nucleotides, where the receptor binding affinity is dependent upon the presence of the hairpin in the isolated nucleic acid.

In various embodiments of any of the above aspects or any aspect delineated herein, the receptor is expressed on the surface of a cell. In particular various embodiments, the receptor is selected from the list consisting of nucleolin, a human epidermal growth factor receptor 2 (HER2), CD20, a transferrin receptor, an asialoglycoprotein receptor, a thyroid-stimulating hormone (TSH) receptor, a fibroblast growth factor (FGF) receptor, CD3, the interleukin 2 (IL-2) receptor, a growth hormone receptor, an insulin receptor, an acetylcholine receptor, an adrenergic receptor, a vascular endothelial growth factor (VEGF) receptor, a protein channel, cadherin, a desmosome, and a viral receptor. In various embodiments of any of the above aspects or any aspect delineated herein, the receptor is internalized into a mammalian cell by an amount (expressed by %) selected from the group consisting of: at least 10%, at least 50% and at least 80-90%.

In various embodiments of any of the above aspects or any aspect delineated herein, the isolated nucleic acid molecule is cleaved endogenously in a mammalian cell to produce a double-stranded ribonucleic acid (dsRNA) of 19-23 nucleotides in length that reduces target gene expression. In various embodiments of any of the above aspects or any aspect delineated herein, the isolated nucleic acid molecule reduces target gene expression in a mammalian cell in vitro by an amount (expressed by %) selected from the group consisting of: at least 10%, at least 50% and at least 80-90%. In various embodiments of any of the above aspects or any aspect delineated herein, the isolated nucleic acid molecule, when introduced into a mammalian cell, reduces target gene expression in comparison to a reference dsRNA. In various embodiments of any of the above aspects or any aspect delineated herein, the isolated nucleic acid molecule, when introduced into a mammalian cell, reduces target gene expression by at least 70% when transfected into the cell at a concentration selected from the group consisting of: 1 nM or less, 200 pM or less, 100 pM or less, 50 pM or less, 20 pM or less and 10 pM or less.

In various embodiments of any of the above aspects or any aspect delineated herein, Dicer cleavage results in unfolding of the aptamer by an amount (expressed by %) selected from the group consisting of: at least 10%, at least 50% and at least 80-90%. In various embodiments of any of the above aspects or any aspect delineated herein, Dicer cleavage decreases the stability of the isolated nucleic acid molecule by an amount (expressed by %) selected from the group consisting of: at least 10%, at least 50% and at least 80-90%. In various embodiments of any of the above aspects or any aspect delineated herein, Dicer cleavage increases the degradation of the isolated nucleic acid molecule by an amount (expressed by %) selected from the group consisting of: at least 10%, at least 50% and at least 80-90%.

In various embodiments of any of the above aspects or any aspect delineated herein, the isolated nucleic acid contains a modified nucleotide. In various specific embodiments, the modified nucleotide residue is selected from the group consisting of: 2′-O-methyl, 2′-methoxyethoxy, 2′-fluoro, 2′-allyl, 2′-O -[2-(methylamino)-2-oxoethyl], 4′-thio, 4′-CH2-O-2′-bridge, 4′-(CH2)2-O-2′-bridge, 2′-LNA, 2′-amino and 2′-O-(N-methlycarbamate). In various embodiments of any of the above aspects or any aspect delineated herein, the isolated nucleic acid molecule has increased nuclease resistance relative to a reference dsRNA. In various embodiments of any of the above aspects or any aspect delineated herein, Dicer cleavage decreases the nuclease resistance of the isolated nucleic acid molecule by an amount (expressed by %) selected from the group consisting of: at least 10%, at least 50% and at least 80-90%.

In various embodiments of any of the above aspects or any aspect delineated herein, the isolated nucleic acid molecule does not inhibit Dicer.

In various embodiments of any of the above aspects or any aspect delineated herein, the isolated nucleic acid molecule is identified using systematic evolution of ligands by exponential enrichment (SELEX).

In various embodiments of any of the above aspects or any aspect delineated herein, the method further involves contacting the isolated nucleic acid molecule Dicer cleaved nucleic acid molecule with the receptor and determining binding to the receptor. In various embodiments of any of the above aspects or any aspect delineated herein, the method involves systematic evolution of ligands by exponential enrichment (SELEX).

A Dicer substrate molecule covalently attached to a nucleic acid that binds a receptor imparts certain advantages to the DsiRNA molecule, including, e.g., receptor binding, enhanced delivery, enhanced efficacy (including enhanced potency and/or improved duration of effect). It is appreciated that the receptor binding property of a nucleic acid molecule of the invention is a useful method at least for targeting a Dicer substrate to a cell having a receptor on its surface. Such receptors include polypeptide and carbohydrate molecules present on the cell surface. It is further contemplated that, when bound to a cell surface receptor, the aptamer is internalized into the cell, thus delivering the DsiRNA across the plasma membrane and into the cell.

Among the additional advantages of the instant invention, the nucleic acid molecules suitable for systemic use in vivo normally require very high levels of chemical modification in the receptor binding region and are highly nuclease resistant. They can accumulate and potentially cause detrimental effects due to the function of the receptor binding region. If Dicer processing results in the degradation or inactivity of the receptor binding portion of the nucleic acid molecule after internalization, then off target effects from the receptor binding portion should be minimized Another advantage of the invention is that it creates a molecule that can be made from one or two polynucleotide strands. Indeed, the aptamers of the invention are suited for high throughput, small scale synthesis to meet research needs as well as large scale manufacturing for therapeutic applications. A potential advantage of the invention is an increase the nuclease resistance of the DsiRNA because of its association with the chemically modified receptor binding region. Increased nuclease resistance allows a reduction in the extent of unnatural chemical modifications normally required on the DsiRNA.

Thus, in certain aspects, the instant invention allows for design of RNA inhibitory molecules possessing new properties and enhanced efficacies compared to previously described RNA inhibitory molecules, thereby allowing for generation of dsRNA molecules possessing enhanced efficacy, delivery, pharmacokinetic, pharmacodynamic and biodistribution attributes, as well as improved ability.

The invention provides compositions useful in RNAi for inhibiting gene expression and provides methods for their use. In addition, the invention provides RNAi compositions and methods designed to enhance delivery, resistance to nucleases (e.g., serum nucleases), cellular targeting, and intracellular uptake, and decrease toxicity. Additionally, various embodiments of the invention are suited for high throughput, small scale synthesis to meet research needs as well as large scale manufacturing for therapeutic applications. These and other advantages of the invention, as well as additional inventive features, will be apparent from the description of the invention provided herein.

### BRIEF DESCRIPTION OF THE DRAWINGS

FIG. 1 depicts a schematic representation of the structure and predicted Dicer-mediated processing of a Dicer substrate aptamer formed by a polynucleotide strand to yield a RISC-active duplex. A Dicer substrate aptamer adopts a secondary and/or tertiary structure that is capable of binding a receptor. The dsRNA stabilizes the secondary and/or tertiary structure of the Dicer substrate aptamer. In accordance with the invention, a Dicer substrate aptamer comprises a Dicer substrate inhibitory RNA molecule (“DsiRNA”). Arrows denote Dicer cleavage site. Each of the two strands of the DsiRNA are each connected to one of the 5′ and 3′ terminal ends of the aptamer. Dicer-mediated processing of a Dicer substrate aptamer forms a RISC-active duplex and an unfolded aptamer. Because the Dicer site is within the required stem, cleavage by Dicer within the required stem results in unfolding of the aptamer. The unfolded aptamer does not bind a receptor and is nuclease-labile. Preferably the Dicer substrate of the Dicer substrate aptamer is 21-25 bp long and has a 3′ terminal structure that orients Dicer (e.g., 3′ overhang) to ensure formation of RISC-active duplexes directed to a target gene.

FIG. 2 depicts a schematic representation of the structure and predicted Dicer-mediated processing of a Dicer substrate aptamer formed by two polynucleotide strands to yield a RISC-active duplex. A Dicer substrate aptamer adopts a secondary and/or tertiary structure that is capable of binding a receptor. The dsRNA stabilizes the secondary and/or tertiary structure of the Dicer substrate aptamer. In accordance with the invention, a Dicer substrate aptamer comprises a Dicer substrate inhibitory RNA molecule (“DsiRNA”). Arrows denote Dicer cleavage site. Each of the two strands of the Dicer substrate are connected to one of the 5′ and 3′ terminal ends of the aptamer. Dicer-mediated processing of a Dicer substrate aptamer forms a RISC-active duplex and an unfolded aptamer. Because the Dicer site is within the required stem, cleavage by Dicer within the required stem results in unfolding of the aptamer into aptamer pieces. The unfolded aptamer does not bind a receptor and is nuclease-labile. Preferably the DsiRNA of the Dicer substrate aptamer is 21-25 bp long and has a 3′ terminal structure that orients Dicer (e.g., 3′ overhang) to ensure formation of RISC-active duplexes directed to a target gene.

FIGS. 3A-3C depict three embodiments of the Dicer substrate aptamers of the invention formed by a polynucleotide strand. FIG. 3A depicts a schematic representation of a Dicer substrate aptamer where the double stranded region formed by the 5′ terminus and the 3′ terminus has a blunt end. FIG. 3B depicts a schematic representation of a Dicer substrate aptamer where the double stranded region formed by the 5′ terminus and the 3′ terminus has a 3′ overhang (e.g., a 2 nt 3′ overhang). The Dicer substrate aptamer depicted in FIG. 3C may also be referred to as an “asymmetric” Dicer substrate aptamer in reference to the 3′ overhang. FIG. 3B depicts a schematic representation of a Dicer substrate aptamer where the double stranded region formed by the 5′ terminus and the 3′ terminus has mismatched base pairs (e.g., 1-2). The Dicer substrate aptamer depicted in FIG. 3C may also be referred to as “frayed” in reference to the terminal mismatched bases.

FIGS. 4A-4C depict three embodiments of the Dicer substrate aptamers of the invention formed by two polynucleotide strands. FIG. 4A depicts a schematic representation of a Dicer substrate aptamer where the double stranded region formed by the 5′ terminus of a first strand and the 3′ terminus of a second strand has a blunt end. FIG. 4B depicts a schematic representation of a Dicer substrate aptamer where the double stranded region formed by the 5′ terminus of a first strand and the 3′ terminus of a second strand has a 3′ overhang (e.g., a 2 nt 3′ overhang). The Dicer substrate aptamer depicted in FIG. 4B may also be referred to as an “asymmetric”

Dicer substrate aptamer in reference to the 3′ overhang. FIG. 4C depicts a schematic representation of a Dicer substrate aptamer where the double stranded region formed by the 5′ terminus of a first strand and the 3′ terminus of a second strand has mismatched base pairs (e.g., 1-2). The Dicer substrate aptamer depicted in FIG. 4C may also be referred to as “frayed” in reference to the terminal mismatched bases.

FIG. 5 depicts a schematic representation of a method of making a Dicer substrate aptamer of the invention formed by a polynucleotide strand. The method involves contacting a Dicer substrate aptamer with a receptor, isolating the Dicer substrate aptamer bound to the receptor, and contacting the isolated Dicer substrate aptamer with Dicer enzyme. A Dicer substrate aptamer has the property that Dicer cleavage of the nucleic acid molecule in the double-stranded region reduces the ability of the aptamer to bind selectively to the receptor. A Dicer substrate aptamer is capable of being processed by Dicer, including in the presence of the receptor. This method may be incorporated into a selection scheme to identify a Dicer substrate aptamer. As an additional step to such a scheme, the products of the Dicer cleavage (i.e., the receptor binding region or aptamer) may additionally be selected for receptor binding. In this selection, the Dicer cleavage products that do not bind the receptor identify Dicer substrate aptamers. The methods described herein may employ one or more selections using systematic evolution of ligands by exponential enrichment (SELEX).

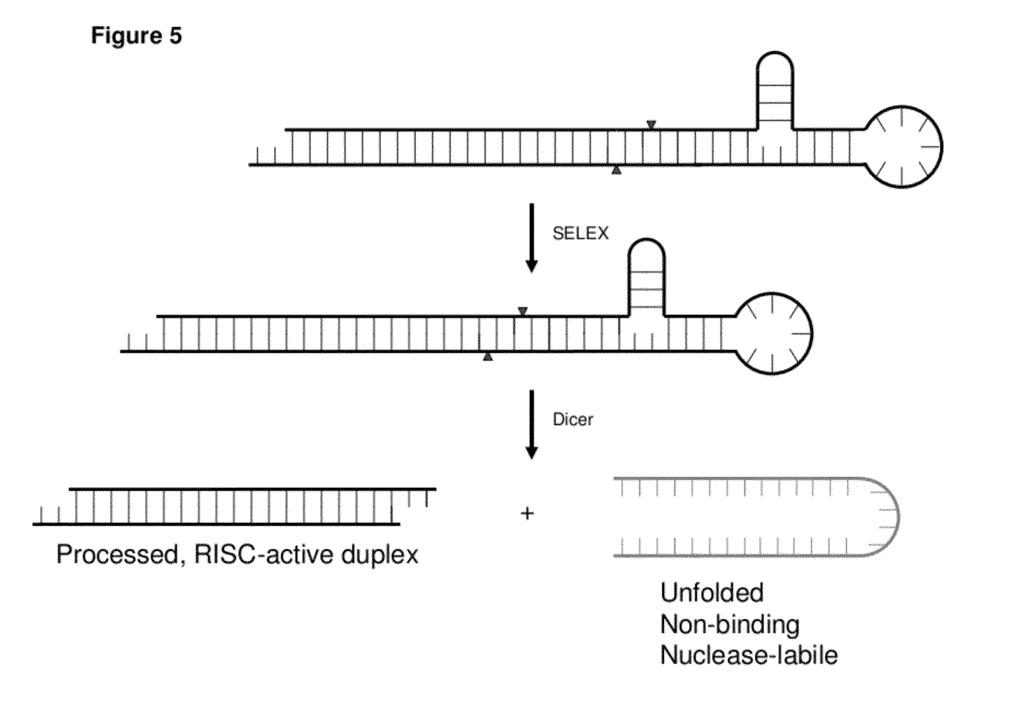

FIG. 6 depicts a schematic representation of a method of making a Dicer substrate aptamer of the invention formed by two polynucleotide strands. The method involves contacting a Dicer substrate aptamer with a receptor, isolating the Dicer substrate aptamer bound to the receptor, and contacting the isolated Dicer substrate aptamer with Dicer enzyme. A Dicer substrate aptamer has the property that Dicer cleavage of the nucleic acid molecule double-stranded region reduces the ability of the aptamer to bind selectively to the receptor. In this embodiment, a Dicer substrate aptamer is capable of being processed by Dicer, including in the presence of the receptor. The methods described herein may employ one or more selections using systematic evolution of ligands by exponential enrichment (SELEX).

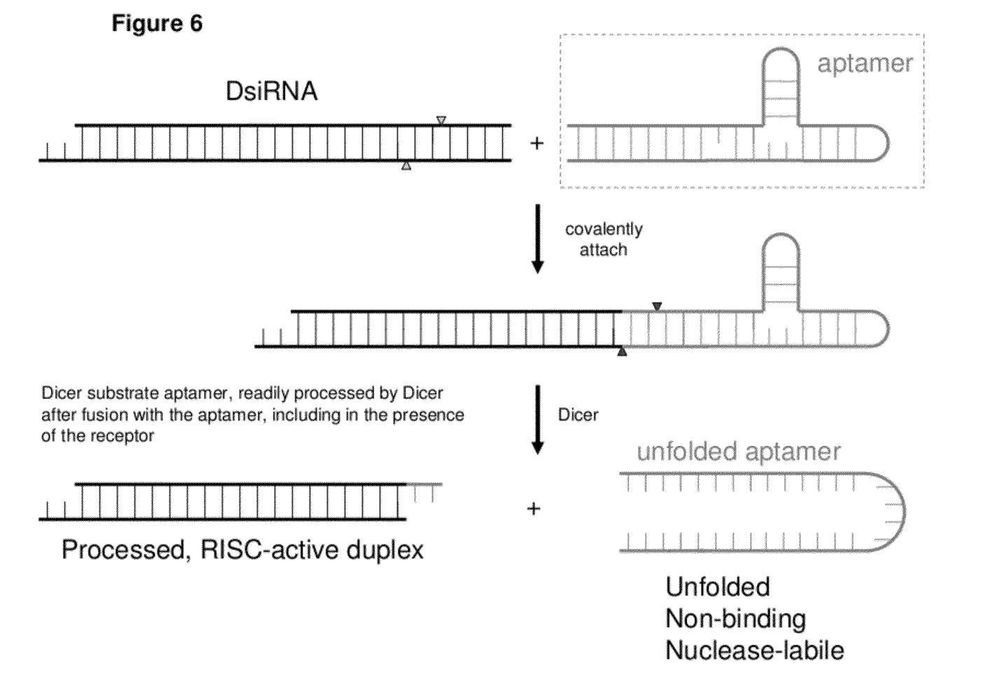

FIG. 7 depicts a schematic representation of a method of making a Dicer substrate aptamer. A mixture of candidate Dicer substrate aptamers are generated, and systematic evolution of ligands by exponential enrichment (SELEX) is performed on the mixture. Candidate Dicer substrate aptamers which bind the receptor in SELEX are selected. Candidate Dicer substrate aptamers that bind in SELEX are then exposed to Dicer enzyme. Candidate Dicer substrate aptamers that are cleaved by Dicer enzyme are selected. SELEX with the same receptor is again performed on the Dicer generated cleavage products of the remaining candidate Dicer substrate aptamers. Dicer generated cleavage products which do not bind the receptor in SELEX correspond to Dicer substrate aptamers. Thus, this method identifies Dicer substrate aptamers which bind a receptor, but do not bind a receptor after Dicer cleavage. Without limitation, a Dicer substrate aptamer substrate identified by this method may be formed from one or two polynucleotide strands.

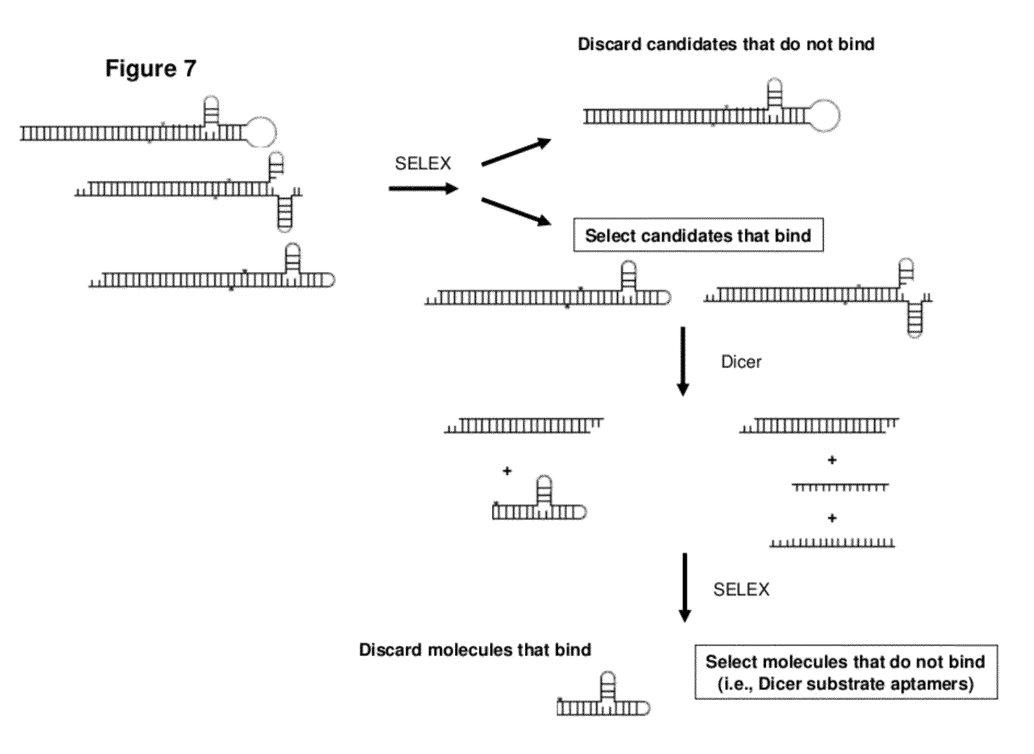

### DETAILED DESCRIPTION

It is appreciated that nucleic acid molecules containing a region that binds to a receptor are useful for attaching double-stranded ribonucleic acids (dsRNAs), including Dicer substrate siRNAs (DsiRNAs). Double stranded nucleic acid molecules having strand lengths in the range of 25-35 nucleotides in length that additionally have a receptor binding region either at or near the 3′ terminus of the sense strand of the antisense strand and at or near the 5′ terminus of the sense strand of the antisense strand are effective RNA interference molecules. That is, the strands of the dsRNA and the strand or strands forming the receptor binding region share a backbone (e.g., a 5′-3′ phosphodiester backbone). Indeed, the instant invention relates to the inclusion of a Dicer substrate siRNA (“DsiRNAs”) that is excised via Dicer enzyme cleavage from a nucleic acid region that binds a receptor, resulting in an effective inhibitory molecule.

The present invention is directed to nucleic acid compositions that contain double stranded RNA (“dsRNA”) and a receptor binding region capable of enhancing the delivery and/or biodistribution or targeting of a dsRNA to a cell and adding further functionality and/or enhancing, e.g. pharmacokinetics or pharmacodynamics of such molecules as compared to dsRNA molecules that do not comprise a receptor binding region as described herein. The present invention is also directed to methods of preparing a nucleic acid molecule comprising a dsRNA and a receptor binding region that is capable of reducing the level and/or expression of genes in vivo or in vitro. In one aspect, the nucleic acid molecules of the invention are useful for delivering Dicer substrate RNAs (“DsiRNAs”). The compositions and methods involve contacting a cell with a nucleic acid molecule of the invention in an amount effective to reduce expression of a target gene in a cell.

The nucleic acid molecules of the invention adopt conformations allowing them to bind to other molecules, such as polypeptides, specifically and with high affinity via non-Watson-Crick interactions. The nucleic acid molecules comprising a dsRNA and a receptor binding region are prepared from one or two polynucleotide strands. It is appreciated that this structure facilitates high throughput, small scale synthesis to meet research needs as well as large scale manufacturing for therapeutic applications. Specifically, nucleic acid molecules comprising a polynucleotide strand 53-142 nucleotides in length, that forms a double-stranded region of at least 21-25 base pairs, which contains at least 19 nucleotides complementary to a target RNA, are effective RNA interference molecules. Additionally, nucleic acid molecules comprising two polynucleotide strand 33-121 nucleotides in length, that form a double-stranded region of at least 21-25 base pairs, which contains at least 19 nucleotides complementary to a target RNA, are effective RNA interference molecules. The Dicer substrate siRNA (“DsiRNAs”) is excised from the nucleic acid molecule of the invention via Dicer enzyme cleavage, resulting in an effective inhibitory molecule. In certain embodiments of the invention, the strand(s) comprising a dsRNA are covalently attached to a nucleic acid aptamer via a nucleic acid backbone (e.g., a 5′-3′ phosphodiester backbone).

Nucleic molecules of the invention containing a Dicer substrate and a receptor-binding region impart certain advantages to the use of the Dicer substrate molecule, including, e.g., enhanced efficacy (including enhanced potency and/or improved duration of effect), receptor binding, and other attributes associated with a nucleic acid aptamer of a given function. The receptor binding property is useful at least for targeting a Dicer substrate to a cell having a receptor on its surface. Such receptors include polypeptide and carbohydrate molecules present on the cell surface either normally or as a result of a pathological condition. It is further contemplated that a cell surface receptor bound to the nucleic acid molecule of the invention is internalized into the cell, thus delivering the Dicer substrate across the plasma membrane and into the cell.

Among the additional advantages of the instant invention, nucleic acid molecules suitable for systemic use in vivo normally require very high levels of chemical modification and are highly nuclease resistant. They can accumulate and potentially cause detrimental effects due to the receptor binding function. Dicer processing results in the degradation or inactivity of the receptor binding region of the nucleic acid molecule after internalization, thus minimizing off target effects. Another advantage of the nucleic acid molecules of the invention is the creation of a molecule with a significantly lower total molecular weight than a “conventional” Dicer substrate (DsiRNA) conjugated to an aptamer by other means. A potential advantage of the invention is an increase the nuclease resistance of the aptamer and the Dicer substrate. Increased nuclease resistance allows a reduction in the extent of unnatural chemical modifications normally required on an aptamer.

Thus, in certain aspects, the instant invention allows for design of RNA inhibitory molecules possessing new properties and enhanced efficacies compared to previously described RNA inhibitory molecule, thereby allowing for generation of dsRNA-containing aptamer molecules possessing enhanced efficacy, delivery, pharmacokinetic, pharmacodynamic and biodistribution attributes, as well as improved ability.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

As used herein, the term “receptor” refers to a specific cell surface molecule, e.g., a marker. Receptors include without limitation proteins, glycoproteins, channels, cadherins, desmosomes, internal proteins inappropriately expressed on cell surfaces, viral or other pathogen markers expressed or displayed on the cell surfaces. For example, specific receptors include nucleolin, a human epidermal growth factor receptor 2 (HER2), CD20. The invention provides compositions and methods for identifying a nucleic acid molecule containing a dsRNA that binds to any type of molecule or marker displayed on the cell surface, whether the presence of the molecule or marker on the cell surface is normal or a result of a pathological condition.

As used herein, “specifically binds” or “selectively binds” means via hydrogen bonding or electrostatic attraction to a receptor of interest, but not to most other molecules. The secondary and/or tertiary structure of a nucleic acid molecule may contribute to the specific binding of a nucleic acid molecule and a receptor. “Specific binding” is determined by a binding assay known in the art and as defined herein (See for example US20080064092 and US2009004174). The nucleic acid molecule preferably binds the receptor with an affinity in the micromolar range (1-100 μM) and more preferably with an affinity in the nanomolar to picomolar range (1-100 nM affinity and 1-100 pM affinity). In one embodiment, specific binding is determined by comparing the binding of a nucleic acid molecule containing a dsRNA and a receptor binding region to the stated, corresponding receptor to the binding of the nucleic acid molecule containing a dsRNA and a receptor binding region to other receptors, wherein all receptors are present in a mixture. An increase, as defined herein, in binding to the stated receptor, as compared to other receptors, is indicative of specific binding.

A “target cell” means any cell as defined herein, for example a cell derived from or present in any organ including but not limited to the brain, the adrenal or other sites outside the brain (e.g., an extracranial site) such as for example, the kidney, the liver, the pancreas, the heart, the spleen, the gastrointestinal (GI) tract (e.g., stomach, intestine, colon), the eyes, the lungs, skin, adipose, muscle, lymph nodes, bone marrow, the urinary and reproductive systems (ovary, breasts, testis, prostrate), placenta, blood cells and a combination thereof.

“Delivery” of a Dicer substrate aptamer or nucleic acid of the invention is assessed by internalization or uptake assays described hereinbelow.

As used herein, the term “nucleic acid” refers to deoxyribonucleotides, ribonucleotides, or modified nucleotides, and polymers thereof in single- or double-stranded form. The term “polynucleotide” refers to deoxyribonucleotides, ribonucleotides, or modified nucleotides, and polymers thereof in single-stranded form. The terms encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

As used herein, “nucleotide” is used as recognized in the art to include those with natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see, e.g., Usman and McSwiggen, supra; Eckstein, et al., International PCT Publication No. WO 92/07065; Usman et al, International PCT Publication No. WO 93/15187; Uhlman & Peyman, supra, all are hereby incorporated by reference herein). There are several examples of modified nucleic acid bases known in the art as summarized by Limbach, et al, Nucleic Acids Res. 22:2183, 1994. Some of the non-limiting examples of base modifications that can be introduced into nucleic acid molecules include, hypoxanthine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, and others (Burgin, et al., Biochemistry 35:14090, 1996; Uhlman & Peyman, supra). By “modified bases” in this aspect is meant nucleotide bases other than adenine, guanine, cytosine and uracil at 1′ position or their equivalents.

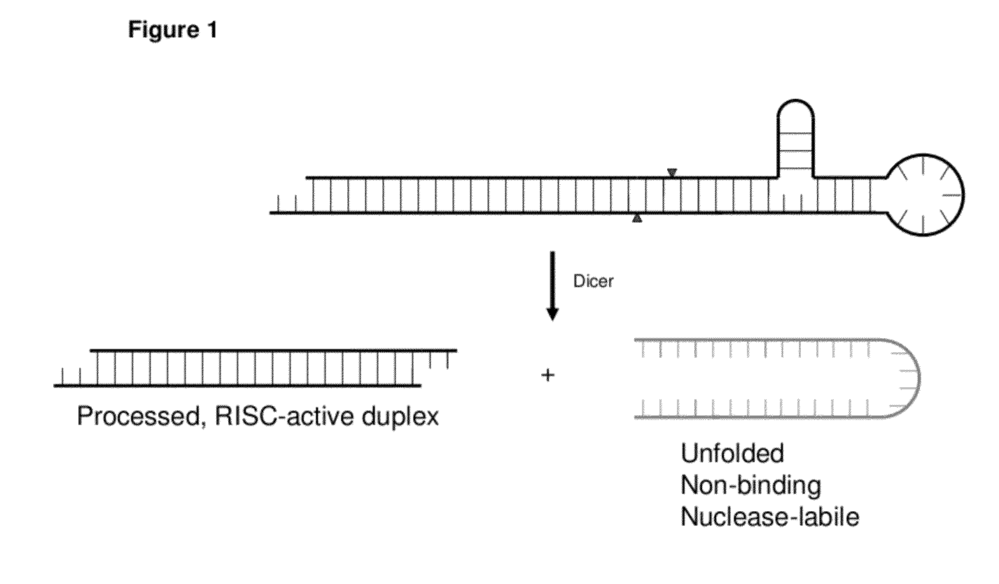

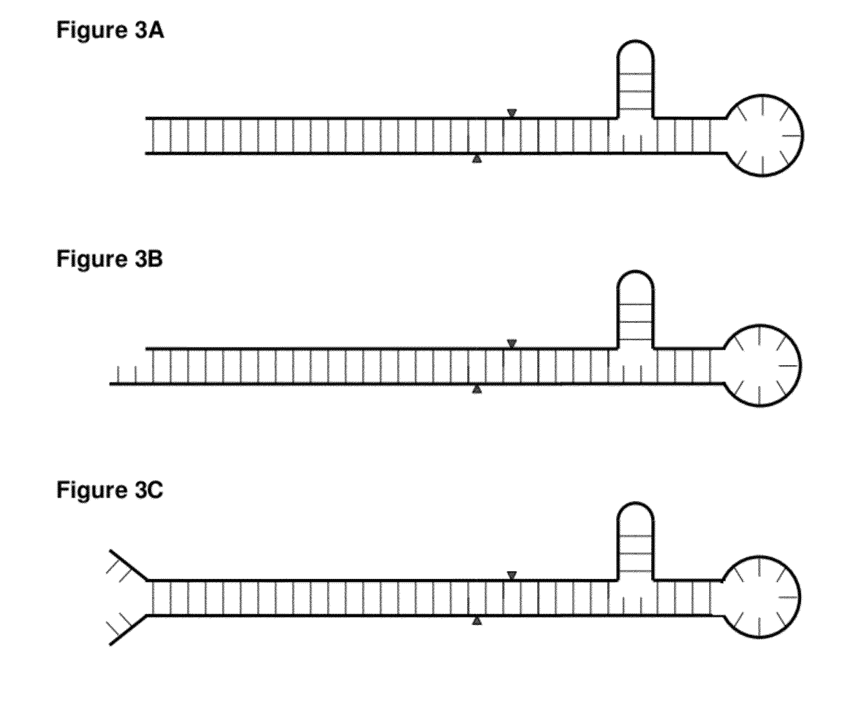

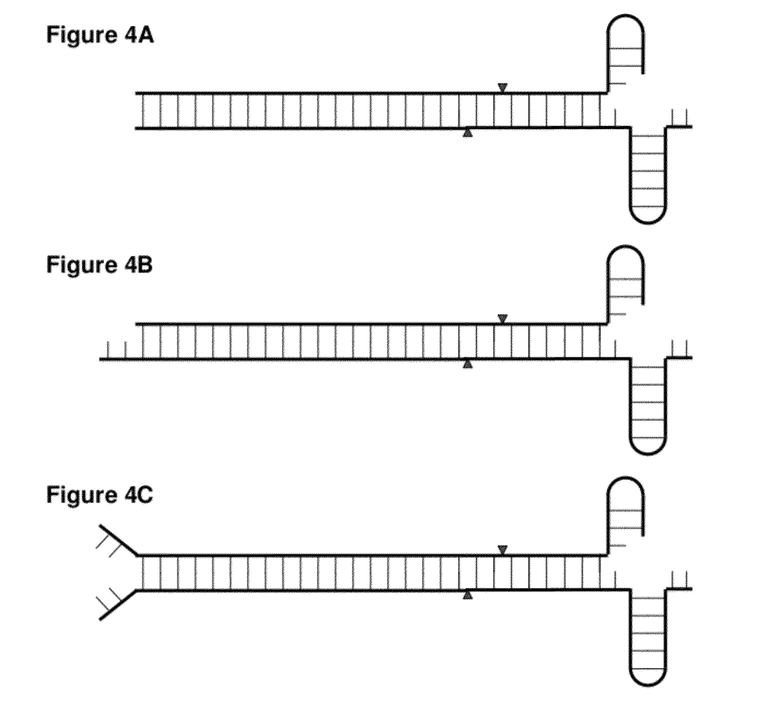

As used herein, a “double-stranded ribonucleic acid” or “dsRNA” is a molecule comprising two oligonucleotide strands which form a duplex and contain at least 4 consecutive ribonucleotides on at least one oligonucleotide strand. A double stranded RNA which is a Dicer substrate (DsiRNA) is of a length and structure sufficient to be susceptible to Dicer cleavage so as to produce a 21-23 bp small inhibitory double stranded RNA. A dsRNA may contain ribonucleotides, deoxyribonucleotides, modified nucleotides, and combinations thereof. The double-stranded NAs of the instant invention are substrates for proteins and protein complexes in the RNA interference pathway, e.g., Dicer and RISC. Exemplary structures of nucleic acid molecules according to the invention containing a dsRNA Dicer substrate and an aptamer are shown in FIGS. 1-4. Such structures characteristically comprise a duplex region comprising RNA residues that is capable of functioning as a Dicer substrate siRNA (DsiRNA) and a receptor binding region, which is located at a position 3′ of the projected Dicer cleavage site of the first strand of the DsiRNA/NA molecule, and is at a position 5′ of the projected Dicer cleavage site of the second strand of the DsiRNA/NA molecule.

As used herein, “Dicer substrate aptamer”, (isolated nucleic acid molecules according to the invention), in the context of the invention, refers to a synthetic nucleic acid molecule comprising at least one double-stranded portion, and at least one single-stranded loop of at least 3 unpaired nucleotides. Functionally, the synthetic nucleic acid molecule comprises a double stranded region which is susceptible to cleavage by Dicer (i.e., a “Dicer substrate portion”) and a region of both paired and unpaired bases which forms a secondary and tertiary structure permitting the entire molecule to selectively bind to a receptor, and which upon cleavage by dicer loses its ability to selectively bind the receptor. A “Dicer substrate aptamer”, by virtue of being a Dicer substrate includes a region which is susceptible to cleavage by Dicer (preferably a mammalian dicer enzyme such as human dicer). The Dicer cleavage susceptible portion (or region) may be at least 21 base pairs and at most 25 base pairs in length containing at least 19 base pairs complementary to a target RNA, i.e., a “small inhibitory RNA”. The aptamer function of the molecule comprises a nucleic acid portion (or region) that specifically binds a receptor. It will be appreciated that it is the entire molecule which provides the two functions of serving as a Dicer substrate and an aptamer, and therefore these distinct functions may not be separable into two distinct regions of the molecule. To the extent that the Dicer substrate and aptamer functions of the molecule are assignable to a given secondary or tertiary structure of the molecule, these structures may overlap and thus participate in more than one of the two functions.

For example, it is known that Dicer enzyme requires a substantially double stranded region having at least one double stranded terminus composed of a 5′ terminal nucleotide and a 3′ terminal nucleotide (which is not required to be co-extensive and thus may be unpaired or may include a 3′ terminal overhang), where the substantially double stranded region must be of a sufficient length for Dicer enzyme cleavage to produce a cleave duplex of at least 19 and preferably 21-23 base pairs. Therefore, the Dicer substrate function of the molecule will be formed from a substantially double stranded structure where each strand is at least 24 nucleotides, and most preferably at least 25 nucleotides.

The aptamer function of the molecule is provided by a nucleic acid having sufficient secondary and/or tertiary structure to specifically bind a receptor with an affinity of at least 1μm. Aptamer structure includes both single stranded and double stranded regions, where the double stranded regions are believed to confer stability on the overall structure of the molecule, particularly with respect to the single stranded regions.

To the extent that the Dicer cleavage susceptible function requires a substantially double stranded (substantially complementary) region and at least one double stranded terminus having a 5′ and a 3′ terminal nucleotide, and the receptor binding function requires secondary and tertiary structure composed of double stranded and single stranded regions, the structures may participate in both functions. For example, the double stranded portion(s) of the molecule may overlap to the extent that some or all of the base pairs participate in thereceptor binding function as well as the Dicer cleavage susceptible function. Alternatively, there may be at least two distinct double stranded regions of the molecule, at least one of which participates in each of the two stated functions.

The Dicer substrate aptamers can be formed by one or by two polynucleotide strands, where the nucleic acid molecule formed by one strand contains a single 5′ terminus and a single 3′ terminus, or the nucleic acid molecule formed by two polynucleotide strands contains a 5′ and a 3′ terminus for each of the two strands.

In various embodiments, the molecule formed by one polynucleotide strand is 53-142 nucleotides in length and the 5′ terminus and 3′ terminus form a dsRNA duplex, i.e., a double-stranded region of at least 21-25 base pairs (see, for example, FIG. 1). The dsRNA duplex comprises at least 19 nucleotides on the antisense strand complementary to a target RNA sense strand.

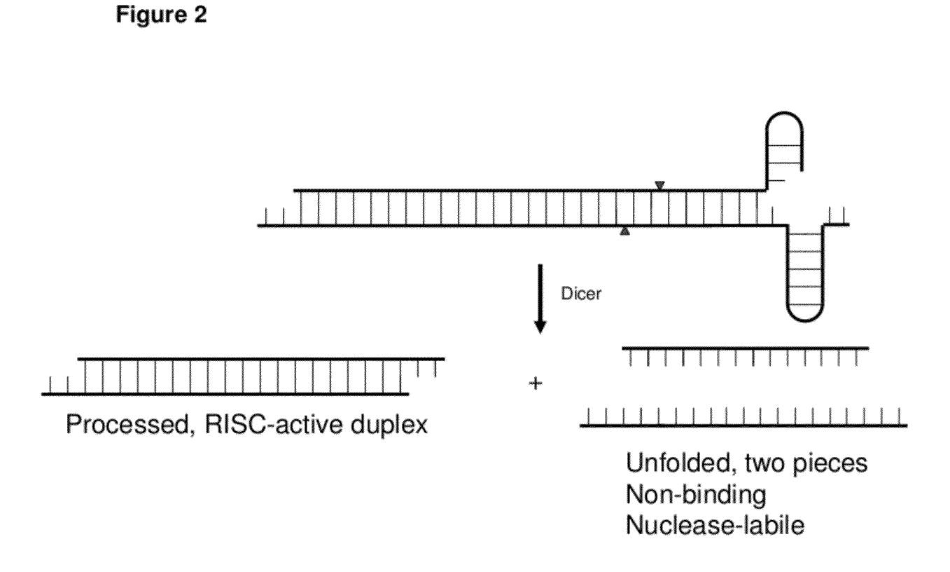

In other embodiments, the nucleic acid molecule comprises two polynucleotide strands, each strand being 33-121 nucleotides in length. See, for example, FIG. 2; where,for example, the molecules depicted in FIG. 2 are oriented such that the top strand of each molecule of FIG. 2 is oriented 5′ to 3′ from left to right (and may be the “sense” strand), and the bottom strand of each molecule of FIG. 2 is oriented 3′ to 5′ from left to right (and may be the “antisense” strand). Thus, the 5′ portion of the polynucleotide strand containing the sense strand and the 3′ portion of the polynucleotide strand containing the antisense strand form a dsRNA or double-stranded region of at least 21-25 base pairs, which contains at least 19 nucleotides complementary to a target RNA.

In the embodiments depicted in FIGS. 1 and 2, the entire molecule comprises at least one substantially double stranded portion and at least one single stranded portion, where the at least one double stranded portion comprises at least 4 consecutive base pairs which are 2′-hydroxyl pentofuranosyl paired nucleosides, preferably paired ribonucleoside residues. The at least 4 consecutive 2′-hydroxyl pentofuranosyl paired nucleosides may be present in any duplex portion of the entire molecule; it is preferred that these consecutive paired 2′-hydroxyl pentofuranosyl nucleosides are present in the double-stranded region which serves as a substrate for Dicer, most preferably they constitute the nucleotide pairs cleaved by Dicer (Dicer cleavage sites depicted as filled arrowheads in FIGS. 1 and 2). The entire molecule also may include at least 5, 6, 7, 8, 9, 10, 11, 12 or up to 21-25, consecutive 2′-hydroxyl pentofuranosyl paired nucleosides; it is preferred that the dicer substrate portion of the molecule comprise consecutive 2′-hydroxyl pentofuranosyl paired nucleosides. It is preferred that the 2′-hydroxyl pentofuranosyl nucleoside are ribonucleotides.

The receptor binding function requires at least one double stranded region and at least one single stranded region, and can form at least one hairpin (stem/loop). The double-stranded region of the molecule that participates in (i.e., is required for) receptor binding (with an affinity of, e.g., at least 100 μm) contains an internally base-paired region comprising at least 4, 5, 6, 7, 8, 9, 10, 11, 12 (preferably no more than 12) consecutive base pairs and a single-stranded region comprising 5, 6, 7, 8, 9, 10, 11, 12, 13, 14, 15, 16, 17, 18, 19, 20 consecutive non-base paired nucleotides, wherein the receptor binding affinity is dependent upon the presence of the at least one double stranded and at least one single stranded region in the nucleic acid.

Dicer cleavage of the Dicer substrate aptamer in the double-stranded region reduces target gene expression in a mammalian cell, and reduces the ability of the nucleic acid molecule to bind specifically to the receptor. A Dicer substrate aptamer also includes a nucleic acid molecule in which two physically distinct molecules, each having a distinct function, are covalently attached. In this embodiment, a Dicer substrate molecule is covalently attached to a known nucleic acid aptamer (i.e., a physically distinct molecule having the ability to bind a defined receptor with high affinity and/or specificity). Such a Dicer substrate aptamer typically contains at least one region comprising at least four ribonucleotides (optionally including modified ribonucleotides) that form a Dicer cleavage susceptible region (as a distinct molecule, referred to as a “DsiRNA”) which, upon cleavage by Dicer, produces a small inhibitory double stranded molecule (“siRNA”). The region serving the Dicer substrate function may be contemplated as covalently attached to a second region comprising a nucleic acid aptamer serving the receptor binding function.

An isolated nucleic acid molecule according to the invention possesses one or more beneficial properties (such as, for example, increased efficacy, e.g., increased potency and/or duration of DsiRNA activity, function as a recognition domain or means of targeting the nucleic acid molecule to a specific location, for example, when administered to cells in culture or to a subject, functioning as an extended region for improved attachment of functional groups, payloads, detection/detectable moieties, functioning as an extended region that allows for more desirable modifications and/or improved spacing of such modifications, etc.). The nucleic acid aptamer may also include modified or synthetic nucleotides and/or modified or synthetic deoxyribonucleotides.

In certain embodiments, a Dicer substrate aptamer of the invention comprises at least one region (“aptamer region”), located (referring to FIGS. 1 and 2) downstream of (or 3′ of) the projected Dicer cleavage site of the top strand (and correspondingly 5′ of the projected Dicer cleavage site of the bottom strand), having a secondary and/or tertiary structure. The structure of the aptamer region may be selected for a functional process by SELEX or another in vitro selection process.

In some embodiments, the first and second strands of the Dicer substrate share the same nucleic acid backbone with the aptamer (e.g., the 3′ end of the first strand of the Dicer substrate portion is connected by a 3′-5′ phosphodiester linkage to the 5′ end of the nucleic acid aptamer portion and the 3′ end of the nucleic acid aptamer portion is connected by a 3′-5′ phosphodiester linkage to the 5′ end of the second strand of the Dicer substrate portion—see, e.g., FIG. 2).

In other embodiments, the first and second strands of Dicer substrate portion of the molecule share a backbone with two polynucleotides which form the aptamer (e.g., the 3′ end of the first strand of the Dicer substrate is connected by a 3′-5′ phosphodiester linkage to the 5′ end of the nucleic acid aptamer, the 3′ end of the nucleic acid aptamer is connected by a 3′-5′ phosphodiester linkage to the 5′ end of the second strand of the Dicer substrate, and the two strands form a duplex in the Dicer substrate region and, although discontinuous, adopt appropriate secondary and/or tertiary structure in the aptamer region).

As used herein, “duplex” refers to a double helical structure formed by the interaction of two single stranded nucleic acids. According to the present invention, a duplex may contain first and second strands which are sense and antisense, or which are target and antisense, or which are simply first and second strands. The duplex may consist of one strand, if the sense and antisense, or target and antisense strand are joined. A duplex is typically formed by the pairwise hydrogen bonding of bases, i.e., “base pairing”, between two single stranded nucleic acids which are oriented antiparallel with respect to each other. Base pairing in duplexes generally occurs by Watson-Crick base pairing, e.g., guanine (G) forms a base pair with cytosine (C) in DNA and RNA (thus, the cognate nucleotide of a guanine deoxyribonucleotide is a cytosine deoxyribonucleotide, and vice versa), adenine (A) forms a base pair with thymine (T) in DNA, and adenine (A) forms a base pair with uracil (U) in RNA. Conditions under which base pairs can form include physiological or biologically relevant conditions (e.g., intracellular: pH 7.2, 140 mM potassium ion; extracellular pH 7.4, 145 mM sodium ion). Furthermore, duplexes are stabilized by stacking interactions between adjacent nucleotides. As used herein, a duplex may be established or maintained by base pairing or by stacking interactions. A duplex is formed by two complementary nucleic acid strands, which may be substantially complementary or fully complementary, or two complementary regions of a single nucleic strand, which may be substantially complementary or fully complementary.

By “complementary” or “complementarity” is meant that a nucleic acid can form hydrogen bond(s) with another nucleic acid sequence by either traditional Watson-Crick or Hoogsteen base pairing. In reference to the nucleic acid molecules of the present disclosure, the binding free energy for a nucleic acid molecule with its complementary sequence is sufficient to allow the relevant function of the nucleic acid to proceed, e.g., RNAi activity, secondary/tertiary aptamer structure formation. Determination of binding free energies for nucleic acid molecules is well known in the art (see, e.g., Turner, et al., CSH Symp. Quant. Biol. LII, pp. 123-133, 1987; Frier, et al., Proc. Nat. Acad. Sci. USA 83:9373-9377, 1986; Turner, et al., J. Am. Chem. Soc. 109:3783-3785, 1987). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, or 10 nucleotides out of a total of 10 nucleotides in the first oligonucleotide being based paired to a second nucleic acid sequence having 10 nucleotides represents 50%, 60%, 70%, 80%, 90%, and 100% complementary, respectively). To determine that a percent complementarity is of at least a certain percentage, the percentage of contiguous residues in a nucleic acid molecule that can form hydrogen bonds (e.g., Watson-Crick base pairing) with a second nucleic acid sequence is calculated and rounded to the nearest whole number (e.g., 12, 13, 14, 15, 16, or 17 nucleotides out of a total of 23 nucleotides in the first oligonucleotide being based paired to a second nucleic acid sequence having 23 nucleotides represents 52%, 57%, 61%, 65%, 70%, and 74%, respectively; and has at least 50%, 50%, 60%, 60%, 70%, and 70% complementarity, respectively). As used herein, “substantially complementary” refers to complementarity between the strands such that they are capable of hybridizing under biological conditions. Substantially complementary sequences have 60%, 70%, 80%, 90%, 95%, or even 100% complementarity. Additionally, techniques to determine if two strands are capable of hybridizing under biological conditions by examining their nucleotide sequences are well known in the art.

The first and second strands of the Dicer substrate region of the nucleic acid molecule of the invention (antisense and sense oligonucleotides) are not required to be completely complementary. In one embodiment, the RNA sequence of the antisense strand contains one or more mismatches or modified nucleotides with base analogs. In an exemplary embodiment, such mismatches occur within the 3′ region of RNA sequence of the antisense strand (e.g., within the RNA sequence of the antisense strand that is complementary to the target RNA sequence that is positioned 5′ of the projected Argonaute 2 (Ago2) cut site within the target RNA). In one aspect, about two mismatches or modified nucleotides with base analogs are incorporated within the RNA sequence of the antisense strand that is 3′ in the antisense strand of the projected Ago2 cleavage site of the target RNA sequence when the target RNA sequence is hybridized.

The use of mismatches or decreased thermodynamic stability (specifically at or near the 3′-terminal residues of sense/5′-terminal residues of the antisense region of siRNAs) has been proposed to facilitate or favor entry of the antisense strand into RISC (Schwarz et al., 2003; Khvorova et al., 2003), presumably by affecting some rate-limiting unwinding steps that occur with entry of the siRNA into RISC. Thus, terminal base composition has been included in design algorithms for selecting active 21 mer siRNA duplexes (Ui-Tei et al., 2004; Reynolds et al., 2004).

In certain embodiments, mismatches (or modified nucleotides with base analogs) can be positioned within a Dicer substrate region of the nucleic acid aptamer at or near the predicted 3′-terminus of the sense strand of the siRNA projected to be formed following Dicer cleavage. In such embodiments, the small end-terminal sequence which contains the mismatch(es) will either be left unpaired with the antisense strand (become part of a 3′-overhang) or be cleaved entirely off the final 21-mer siRNA. In such embodiments, mismatches in the original (non-Dicer-processed) molecule do not persist as mismatches in the final RNA component of RISC. It has been found that base mismatches or destabilization of segments at the 3′-end of the sense strand of Dicer substrate improved the potency of synthetic duplexes in RNAi, presumably by facilitating processing by Dicer (Collingwood et al., 2008).

In some embodiments, one or more mismatches are positioned within a Dicer substrate region of a nucleic acid molecule of the invention at a location within the region of the antisense strand of the Dicer substrate region that hybridizes with the region of the target mRNA that is positioned 5′ of the predicted Ago2 cleavage site within the target mRNA. Optionally, two or more mismatches (“frayed” structure) are positioned within the Dicer substrate region of a nucleic acid molecule of the instant invention within the relatively 3′ region of the antisense strand that hybridizes to a sequence of the target RNA that is positioned 5′ of the projected Ago2 cleavage site of the target RNA (were target RNA cleavage to occur). Inclusion of such mismatches within the Dicer substrate region of a nucleic acid molecule of the instant invention can allow such molecules to exert inhibitory effects that resemble those of naturally-occurring miRNAs, and optionally can be directed against not only naturally-occurring miRNA target RNAs (e.g., 3′ UTR regions of target transcripts) but also against RNA sequences for which no naturally-occurring antagonistic miRNA is known to exist. For example, a nucleic acid molecule of the invention containing a Dicer substrate region possessing mismatched base pairs which are designed to resemble and/or function as miRNAs can be synthesized to target repetitive sequences within genes/transcripts that might not be targeted by naturally-occurring miRNAs (e.g., repeat sequences within the Notch protein can be targeted, where individual repeats within Notch can differ from one another (e.g., be degenerate) at the nucleic acid level, but which can be effectively targeted via a miRNA mechanism that allows for mismatch(es) yet also allows for a more promiscuous inhibitory effect than a corresponding, perfect match siRNA molecule). In such embodiments, target RNA cleavage may or may not be necessary for the mismatch-containing Dicer substrate region of the nucleic acid molecule to exert an inhibitory effect.

Single-stranded nucleic acids that base pair over a number of bases are said to “hybridize.” Hybridization is typically determined under physiological or biologically relevant conditions (e.g., intracellular: pH 7.2, 140 mM potassium ion; extracellular pH 7.4, 145 mM sodium ion). Hybridization conditions generally contain a monovalent cation and biologically acceptable buffer and may or may not contain a divalent cation, complex anions, e.g. gluconate from potassium gluconate, uncharged species such as sucrose, and inert polymers to reduce the activity of water in the sample, e.g. PEG. Such conditions include conditions under which base pairs can form.

Hybridization is measured by the temperature required to dissociate single stranded nucleic acids forming a duplex, i.e., (the melting temperature; Tm). Hybridization conditions are also conditions under which base pairs can form. Various conditions of stringency can be used to determine hybridization (see, e.g., Wahl, G. M. and S. L. Berger (1987) Methods Enzymol. 152:399; Kimmel, A. R. (1987) Methods Enzymol. 152:507). Stringent temperature conditions will ordinarily include temperatures of at least about 30° C., more preferably of at least about 37° C., and most preferably of at least about 42° C. The hybridization temperature for hybrids anticipated to be less than 50 base pairs in length should be 5-10° C. less than the melting temperature (Tm) of the hybrid, where Tm is determined according to the following equations. For hybrids less than 18 base pairs in length, Tm(° C.)=2(# of A+T bases)+4(# of G+C bases). For hybrids between 18 and 49 base pairs in length, Tm(° C,)=81.5+16.6(log 10[Na+])+0.41 (% G+C)−(600/N), where N is the number of bases in the hybrid, and [Na+] is the concentration of sodium ions in the hybridization buffer ([Na+] for 1×SSC=0.165 M). For example, a hybridization determination buffer is shown in Table 1.

TABLE 1

| | | | | | | | To make | |
|---|---|---|---|---|---|---|---|---|
| | | | | | | | 50 mL | |
| | final conc. | | Vender | Cat# | Lot# | m.w./Stock | solution | |
| NaCl | 100 | mM | Sigma | S-5150 | 41K8934 | 5M | 1 | mL |
| KCl | 80 | mM | Sigma | P-9541 | 70K0002 | 74.55 | 0.298 | g |
| MgCl₂ | 8 | mM | Sigma | M-1028 | 120K8933 | 1M | 0.4 | mL |
| sucrose | 2% | w/v | Fisher | BP220- | 907105 | 342.3 | 1 | g |
| | | | | 212 | | | | |
| Tris-HCl | 16 | mM | Fisher | BP1757- | 12419 | 1M | 0.8 | mL |
| | | | | 500 | | | | |
| NaH₂PO₄ | 1 | mM | Sigma | S-3193 | 52H- | 120.0 | 0.006 | g |
| | | | | | 029515 | | | |
| EDTA | 0.02 | mM | Sigma | E-7889 | 110K89271 | 0.5M | 2 | μL |
| H₂O | | | Sigma | W-4502 | 51K2359 | | to 50 | mL |

| | |
|---|---|
| pH = 7.0 | adjust with |
| at 20° C. | HCl |

Useful variations on hybridization conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Antisense to Molecular Cloning Techniques, 1987, Academic Press, New York); and Sambrook et al., Molecular Cloning: A Laboratory Manual, Cold Spring Harbor Laboratory Press, New York.

As used herein, “oligonucleotide strand” or “polynucleotide strand” is a single stranded nucleic acid molecule. An oligonucleotide may comprise ribonucleotides, deoxyribonucleotides, modified nucleotides (e.g., nucleotides with 2′ modifications, synthetic base analogs, etc.) or combinations thereof. Such modified oligonucleotides can be preferred over native forms because of properties such as, for example, enhanced cellular uptake and increased stability in the presence of nucleases.

As used herein, the term “ribonucleotide” encompasses natural and synthetic, unmodified and modified ribonucleotides. Modifications include changes to the sugar moiety, to the base moiety and/or to the linkages between ribonucleotides in the oligonucleotide. As used herein, the term “ribonucleotide” specifically excludes a deoxyribonucleotide, which is a nucleotide possessing a single proton group at the 2′ ribose ring position.

As used herein, the term “deoxyribonucleotide” encompasses natural and synthetic, unmodified and modified deoxyribonucleotides. Modifications include changes to the sugar moiety, to the base moiety and/or to the linkages between deoxyribonucleotide in the oligonucleotide. As used herein, the term “deoxyribonucleotide” also includes a modified ribonucleotide that does not permit Dicer cleavage of a dsRNA molecule, e.g., a 2′-O-methyl ribonucleotide, a phosphorothioate-modified ribonucleotide residue, etc., that does not permit Dicer cleavage to occur at a bond of such a residue.

As used herein, the term “PS-NA” refers to a phosphorothioate-modified nucleotide residue. The term “PS-NA” therefore encompasses both phosphorothioate-modified ribonucleotides (“PS-RNAs”) and phosphorothioate-modified deoxyribonucleotides (“PS-DNAs”).

In certain embodiments, a nucleic acid molecule of the invention comprises at least one duplex region of at least 23 nucleotides in length, within which at least 50% of all nucleotides are unmodified ribonucleotides. As used herein, the term “unmodified ribonucleotide” refers to a ribonucleotide possessing a hydroxyl (—OH) group at the 2′ position of the ribose sugar.

As used herein, “antisense strand” refers to a single stranded nucleic acid molecule which has a sequence complementary to that of a target RNA. When the antisense strand contains modified nucleotides with base analogs, it is not necessarily complementary over its entire length, but must at least hybridize with a target RNA.

As used herein, “sense strand” refers to a single stranded nucleic acid molecule which has a sequence complementary to that of an antisense strand. When the antisense strand contains modified nucleotides with base analogs, the sense strand need not be complementary over the entire length of the antisense strand, but must at least duplex with the antisense strand.

As used herein, “guide strand” refers to a single stranded nucleic acid molecule of a dsRNA or dsRNA-containing molecule, which has a sequence sufficiently complementary to that of a target RNA to result in RNA interference. After cleavage of the dsRNA or dsRNA-containing molecule by Dicer, a fragment of the guide strand remains associated with RISC, binds a target RNA as a component of the RISC complex, and promotes cleavage of a target RNA by RISC. As used herein, the guide strand does not necessarily refer to a continuous single stranded nucleic acid and may comprise a discontinuity, preferably at a site that is cleaved by Dicer. A guide strand is an antisense strand.

As used herein, “target RNA” refers to an RNA that would be subject to modulation guided by the antisense strand, such as targeted cleavage or steric blockage. The target RNA could be, for example genomic viral RNA, mRNA, a pre-mRNA, or a non-coding RNA. The preferred target is mRNA, such as the mRNA encoding a disease associated protein, such as ApoB, Bcl2, Hif-1alpha, Survivin or a p21 ras, such as Ha. ras, K-ras or N-ras.

As used herein, “passenger strand” refers to an oligonucleotide strand of a dsRNA or dsRNA-containing molecule, which has a sequence that is complementary to that of the guide strand. As used herein, the passenger strand does not necessarily refer to a continuous single stranded nucleic acid and may comprise a discontinuity, preferably at a site that is cleaved by Dicer. A passenger strand is a sense strand.

As used herein, “Dicer” refers to an endoribonuclease in the RNase III family that cleaves a dsRNA or dsRNA-containing molecule, e.g., double-stranded RNA (dsRNA) or pre-microRNA (miRNA), into double-stranded nucleic acid fragments about 19-25 nucleotides long, usually with a two-base overhang on the 3′ end. With respect to the nucleic acid molecule of the invention, the duplex formed by a dsRNA region is recognized by Dicer and is a Dicer substrate on at least one strand of the duplex. Dicer catalyzes the first step in the RNA interference pathway, which consequently results in the degradation of a target RNA. The protein sequence of human Dicer is provided at the NCBI database under accession number NP_{—}085124, hereby incorporated by reference.

Dicer “cleavage” is determined as follows (e.g., see Collingwood et al., Oligonucleotides 18:187-200 (2008)). In a Dicer cleavage assay, Dicer substrate aptamers or RNA duplexes (100 μmol) are incubated in 20 μL of 20 mM Tris pH 8.0, 200 mM NaCl, 2.5 mM MgCl₂ with or without 1 unit of recombinant human Dicer (Stratagene, La Jolla, Calif.) at 37° C. for 18-24 hours. Samples are desalted using a Performa SR 96-well plate (Edge Biosystems, Gaithersburg, Md.). Electrospray-ionization liquid chromatography mass spectroscopy (ESI-LCMS) of duplex RNAs pre- and post-treatment with Dicer is done using an Oligo HTCS system (Novatia, Princeton, N.J.; Hail et al., 2004), which consists of a ThermoFinnigan TSQ7000, Xcalibur data system, ProMass data processing software and Paradigm MS4 HPLC (Michrom BioResources, Auburn, Calif.). In this assay, Dicer cleavage occurs where at least 5%, 10%, 20%, 30%, 40%, 50%, 60%, 70%, 80%, 90%, 95%, or even 100% of the Dicer substrate dsRNA, (e.g., as described herein) is cleaved to a shorter dsRNA (e.g., 19-23 bp dsRNA, preferably, 21-23 bp dsRNA). Cleavage is detected by intially labelling one strand via 5′-32P-end labelling using T4 polynucleotide kinase enzyme. Assays are performed as described above and target RNA and the specific RNA cleavage products generated by RNAi are visualized on an autoradiograph of a gel. The percentage of cleavage is determined by PHOSPHOR IMAGER® (autoradiography) quantitation of bands representing intact control RNA or RNA from control reactions without the receptor binding region and the cleavage products generated by the assay.

As used herein, “Dicer cleavage site” refers to the sites at which Dicer cleaves a dsRNA (e.g., the dsRNA region of a nucleic acid molecule of the invention). Dicer contains two RNase III domains which typically cleave both the sense and antisense strands of a dsRNA. The average distance between the RNase III domains and the PAZ domain determines the length of the short double-stranded nucleic acid fragments it produces and this distance can vary (Macrae I, et al. (2006). “Structural basis for double-stranded RNA processing by Dicer”. Science 311 (5758): 195-8.). As shown in FIG. 1, Dicer is projected to cleave certain double-stranded nucleic acids of the instant invention that possess an antisense strand having a 2 nucleotide 3′ overhang at a site between the 21^{st} and 22^{nd} nucleotides removed from the 3′ terminus of the antisense strand, and at a corresponding site between the 21^{st} and 22^{nd} nucleotides removed from the 5′ terminus of the sense strand. The projected and/or prevalent Dicer cleavage site(s) for Dicer substrate aptamer molecules distinct from those depicted in FIGS. 1-4 may be similarly identified via art-recognized methods, including those described in Macrae et al. While the Dicer cleavage event depicted in FIG. 1 generates a 21 nucleotide siRNA, it is noted that Dicer cleavage of a dsRNA (e.g., Dicer substrate) can result in generation of Dicer-processed siRNA lengths of 19 to 23 nucleotides in length. Indeed, in one aspect of the invention that is described in greater detail below, a double stranded DNA region is included within a dsRNA for purpose of directing prevalent Dicer excision of a typically non-preferred 19 mer siRNA.

As used herein, “overhang” refers to unpaired nucleotides, in the context of a duplex having two or four free ends at either the 5′ terminus or 3′ terminus of a dsRNA. In certain embodiments, the overhang is a 3′ or 5′ overhang on the antisense strand or sense strand.

As used herein, “target” refers to any nucleic acid sequence whose expression or activity is to be modulated. In particular embodiments, the target refers to an RNA which duplexes to a single stranded nucleic acid that is an antisense strand in a RISC complex. Hybridization of the target RNA to the antisense strand results in processing by the RISC complex. Consequently, expression of the RNA or proteins encoded by the RNA, e.g., mRNA, is reduced.

As used herein, the term “RNA processing” refers to processing activities performed by components of the siRNA, miRNA or RNase H pathways (e.g., Drosha, Dicer, Argonaute2 or other RISC endoribonucleases, and RNaseH), which are described in greater detail below (see “RNA Processing” section below). The term is explicitly distinguished from the post-transcriptional processes of 5′ capping of RNA and degradation of RNA via non-RISC- or non-RNase H-mediated processes. Such “degradation” of an RNA can take several forms, e.g. deadenylation (removal of a 3′ poly(A) tail), and/or nuclease digestion of part or all of the body of the RNA by any of several endo- or exo-nucleases (e.g., RNase III, RNase P, RNase T1, RNase A (1, 2, 3, 4/5), oligonucleotidase, etc.).

As used herein, “reference” is meant a standard or control. As is apparent to one skilled in the art, an appropriate reference is where only one element is changed in order to determine the effect of the one element.

As used herein, “modified nucleotide” refers to a nucleotide that has one or more modifications to the nucleoside, the nucleobase, pentose ring, or phosphate group. For example, modified nucleotides exclude ribonucleotides containing adenosine monophosphate, guanosine monophosphate, uridine monophosphate, and cytidine monophosphate and deoxyribonucleotides containing deoxyadenosine monophosphate, deoxyguanosine monophosphate, deoxythymidine monophosphate, and deoxycytidine monophosphate. Modifications include those naturally occuring that result from modification by enzymes that modify nucleotides, such as methyltransferases. Modified nucleotides also include synthetic or non-naturally occurring nucleotides. Synthetic or non-naturally occurring modifications in nucleotides include those with 2′ modifications, e.g., 2′-methoxyethoxy, 2′-fluoro, 2′-allyl, 2′-O-[2-(methylamino)-2-oxoethyl], 4′-thio, 4′-CH₂—O-2′-bridge, 4′-(CH₂)₂—O-2′-bridge, 2′-LNA, and 2′-O-(N-methylcarbamate) or those comprising base analogs. In connection with 2′-modified nucleotides as described for the present disclosure, by “amino” is meant 2′-NH₂ or 2′-O—NH₂, which can be modified or unmodified. Such modified groups are described, e.g., in Eckstein et al., U.S. Pat. No. 5,672,695 and Matulic-Adamic et al., U.S. Pat. No. 6,248,878.

In reference to the nucleic acid molecules of the present disclosure, the modifications may exist in patterns on a strand of the region of a nucleic acid molecule of the invention comprising a Dicer substrate. As used herein, “alternating positions” refers to a pattern where every other nucleotide is a modified nucleotide or there is an unmodified nucleotide (e.g., an unmodified ribonucleotide) between every modified nucleotide over a defined length of a strand of the nucleic acid molecule (e.g., 5′-MNMNMN-3′; 3′-MNMNMN-5′; where M is a modified nucleotide and N is an unmodified nucleotide). The modification pattern starts from the first nucleotide position at either the 5′ or 3′ terminus according to any of the position numbering conventions described herein (in certain embodiments, position 1 is designated in reference to the terminal residue of a strand following a projected Dicer cleavage event of a Dicer substrate-containing aptamer of the invention; thus, position 1 does not always constitute a 3′ terminal or 5′ terminal residue of a pre-processed molecule of the invention). The pattern of modified nucleotides at alternating positions may run the full length of the strand, but in certain embodiments includes at least 4, 6, 8, 10, 12, 14 nucleotides containing at least 2, 3, 4, 5, 6 or 7 modified nucleotides, respectively. As used herein, “alternating pairs of positions” refers to a pattern where two consecutive modified nucleotides are separated by two consecutive unmodified nucleotides over a defined length of a strand of the nucleic acid molecule (e.g., 5′-MMNNMMNNMMNN-3′; 3′-MMNNMMNNMMNN-5′; where M is a modified nucleotide and N is an unmodified nucleotide). The modification pattern starts from the first nucleotide position at either the 5′ or 3′ terminus according to any of the position numbering conventions described herein. The pattern of modified nucleotides at alternating positions may run the full length of the strand, but preferably includes at least 8, 12, 16, 20, 24, 28 nucleotides containing at least 4, 6, 8, 10, 12 or 14 modified nucleotides, respectively. It is emphasized that the above modification patterns are exemplary and are not intended as limitations on the scope of the invention.

As used herein, “base analog” refers to a heterocyclic moiety which is located at the 1′ position of a nucleotide sugar moiety in a modified nucleotide that can be incorporated into a nucleic acid duplex (or the equivalent position in a nucleotide sugar moiety substitution that can be incorporated into a nucleic acid duplex). In the nucleic acid molecules of the invention, a base analog is generally either a purine or pyrimidine base excluding the common bases guanine (G), cytosine (C), adenine (A), thymine (T), and uracil (U). Base analogs can duplex with other bases or base analogs in dsRNAs. Base analogs include those useful in the compounds and methods of the invention., e.g., those disclosed in U.S. Pat. Nos. 5,432,272 and 6,001,983 to Benner and US Patent Publication No. 20080213891 to Manoharan, which are herein incorporated by reference. Non-limiting examples of bases include hypoxanthine (I), xanthine (X), 3β-D-ribofuranosyl-(2,6-diaminopyrimidine) (K), 3-β-D-ribofuranosyl-(1-methyl-pyrazolo[4,3-d]pyrimidine-5,7(4H,6H)-dione) (P), iso-cytosine (iso-C), iso-guanine (iso-G), 1-β-D-ribofuranosyl-(5-nitroindole), 1-β-D-ribofuranosyl-(3-nitropyrrole), 5-bromouracil, 2-aminopurine, 4-thio-dT, 7-(2-thienyl)-imidazo[4,5-b]pyridine (Ds) and pyrrole-2-carbaldehyde (Pa), 2-amino-6-(2-thienyl)purine (S), 2-oxopyridine (Y), difluorotolyl, 4-fluoro-6-methylbenzimidazole, 4-methylbenzimidazole, 3-methyl isocarbostyrilyl, 5-methyl isocarbostyrilyl, and 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylindolyl, 4,6-dimethylindolyl, phenyl, napthalenyl, anthracenyl, phenanthracenyl, pyrenyl, stilbenzyl, tetracenyl, pentacenyl, and structural derivates thereof (Schweitzer et al., J. Org. Chem., 59:7238-7242 (1994); Berger et al., Nucleic Acids Research, 28(15):2911-2914 (2000); Moran et al., J. Am. Chem. Soc., 119:2056-2057 (1997); Morales et al., J. Am. Chem. Soc., 121:2323-2324 (1999); Guckian et al., J. Am. Chem. Soc., 118:8182-8183 (1996); Morales et al., J. Am. Chem. Soc., 122(6):1001-1007 (2000); McMinn et al., J. Am. Chem. Soc., 121:11585-11586 (1999); Guckian et al., J. Org. Chem., 63:9652-9656 (1998); Moran et al., Proc. Natl. Acad. Sci., 94:10506-10511 (1997); Das et al., J. Chem. Soc., Perkin Trans., 1:197-206 (2002); Shibata et al., J. Chem. Soc., Perkin Trans., 1: 1605-1611 (2001); Wu et al., J. Am. Chem. Soc., 122(32):7621-7632 (2000); O'Neill et al., J. Org. Chem., 67:5869-5875 (2002); Chaudhuri et al., J. Am. Chem. Soc., 117:10434-10442 (1995); and U.S. Pat. No. 6,218,108.). Base analogs may also be a universal base.

As used herein, “universal base” refers to a heterocyclic moiety located at the 1′ position of a nucleotide sugar moiety in a modified nucleotide, or the equivalent position in a nucleotide sugar moiety substitution, that, when present in a nucleic acid duplex, can be positioned opposite more than one type of base without altering the double helical structure (e.g., the structure of the phosphate backbone). Additionally, the universal base does not destroy the ability of the single stranded nucleic acid in which it resides to duplex to a target nucleic acid. The ability of a single stranded nucleic acid containing a universal base to duplex a target nucleic can be assayed by methods apparent to one in the art (e.g., UV absorbance, circular dichroism, gel shift, single stranded nuclease sensitivity, etc.). Additionally, conditions under which duplex formation is observed may be varied to determine duplex stability or formation, e.g., temperature, as melting temperature (Tm) correlates with the stability of nucleic acid duplexes. Compared to a reference single stranded nucleic acid that is exactly complementary to a target nucleic acid, the single stranded nucleic acid containing a universal base forms a duplex with the target nucleic acid that has a lower Tm than a duplex formed with the complementary nucleic acid. However, compared to a reference single stranded nucleic acid in which the universal base has been replaced with a base to generate a single mismatch, the single stranded nucleic acid containing the universal base forms a duplex with the target nucleic acid that has a higher Tm than a duplex formed with the nucleic acid having the mismatched base.

Some universal bases are capable of base pairing by forming hydrogen bonds between the universal base and all of the bases guanine (G), cytosine (C), adenine (A), thymine (T), and uracil (U) under base pair forming conditions. A universal base is not a base that forms a base pair with only one single complementary base. In a duplex, a universal base may form no hydrogen bonds, one hydrogen bond, or more than one hydrogen bond with each of G, C, A, T, and U opposite to it on the opposite strand of a duplex. Preferably, the universal bases does not interact with the base opposite to it on the opposite strand of a duplex. In a duplex, base pairing between a universal base occurs without altering the double helical structure of the phosphate backbone. A universal base may also interact with bases in adjacent nucleotides on the same nucleic acid strand by stacking interactions. Such stacking interactions stabilize the duplex, especially in situations where the universal base does not form any hydrogen bonds with the base positioned opposite to it on the opposite strand of the duplex. Non-limiting examples of universal-binding nucleotides include inosine, 1-β-D-ribofuranosyl-5-nitroindole, and/or 1-β-D-ribofuranosyl-3-nitropyrrole (US Pat. Appl. Publ. No. 20070254362 to Quay et al.; Van Aerschot et al., An acyclic 5-nitroindazole nucleoside analogue as ambiguous nucleoside. Nucleic Acids Res. 1995 Nov. 11; 23(21):4363-70; Loakes et al., 3-Nitropyrrole and 5-nitroindole as universal bases in primers for DNA sequencing and PCR. Nucleic Acids Res. 1995 Jul. 11; 23(13):2361-6; Loakes and Brown, 5-Nitroindole as an universal base analogue. Nucleic Acids Res. 1994 Oct. 11; 22(20):4039-43).

As used herein, “stem” or “stem structure” refers to a region of internal base pairing comprising 1, 2, 3, 4, 5, 6, 7, or 8 base pairs. The stem may be formed by base pairing of substantially or fully complementary polynucleotide strands or by base pairing of substantially or fully complementary regions of a single polynucleotide strand.

As used herein, “loop” refers to a structure formed by a single strand of a nucleic acid, in which complementary regions that flank a particular single stranded nucleotide region hybridize in a way that the single stranded nucleotide region between the complementary regions is excluded from duplex formation or Watson-Crick base pairing. A loop is a single stranded nucleotide region of any length. Examples of loops include the unpaired nucleotides present in such structures as hairpins, stem loops, or extended loops.

As used herein, “extended loop” in the context of the invention refers to a single stranded loop and in addition 1, 2, 3, 4, 5, 6 or up to 20 base pairs or duplexes flanking the loop. In an extended loop, nucleotides that flank the loop on the 5′ side form a duplex with nucleotides that flank the loop on the 3′ side. An extended loop may participate in a hairpin or stem loop.

As used herein, “tetraloop” in the context of the invention refers to a loop (a single stranded region) consisting of four nucleotides that forms a stable secondary structure that contributes to the stability of an adjacent Watson-Crick hybridized nucleotides. Without being limited to theory, a tetraloop may stabilize an adjacent Watson-Crick base pair by stacking interactions. In addition, interactions among the four nucleotides in a tetraloop include but are not limited to non-Watson-Crick base pairing, stacking interactions, hydrogen bonding, and contact interactions (Cheong et al., Nature 1990 Aug. 16; 346(6285):680-2; Heus and Pardi, Science 1991 Jul. 12; 253(5016):191-4). A tetraloop confers an increase in the melting temperature (Tm) of an adjacent duplex that is higher than expected from a simple model loop sequence consisting of four randomized bases. For example, a tetraloop can confer a melting temperature of at least 55° C. in 10 mM NaHPO₄ to a hairpin comprising a duplex of at least 2 base pairs in length. A tetraloop may contain ribonucleotides, deoxyribonucleotides, modified nucleotides, and combinations thereof. Examples of RNA tetraloops include the UNCG family of tetraloops (e.g., UUCG), the GNRA family of tetraloops (e.g., GAAA), and the CUUG tetraloop. (Woese et al., Proc Natl Acad Sci USA. 1990 November; 87(21):8467-71; Antao et al., Nucleic Acids Res. 1991 Nov. 11; 19(21):5901-5). Examples of DNA tetraloops include the d(GNNA) family of tetraloops (e.g., d(GTTA), the d(GNRA)) family of tetraloops, the d(GNAB) family of tetraloops, the d(CNNG) family of tetraloops, the d(TNCG) family of tetraloops (e.g., d(TTCG)). (Nakano et al. Biochemistry, 41 (48), 14281 -14292, 2002; SHINJI et al. Nippon Kagakkai Koen Yokoshu VOL.78th; NO.2; PAGE. 731 (2000).)

As used herein, “increase” or “enhance” is meant to alter positively by at least 5% compared to a reference in an assay. An alteration may be by 5, 10, 15, 20, 25, 30, 35, 40, 45, 50, 55, 60, 65, 70, 75, 80, 85, 90, 95, 99 or 100% compared to a reference in an assay. An alteration may be by 1, 2, 3, 4, 5, 10, 15, 20, 25, 40, 35, 40, 45, 50, 100, 1000 or 10,000-fold or more compared to a reference in an assay. By “enhance Dicer cleavage,” it is meant that the processing of a quantity of a dsRNA or dsRNA-containing molecule by Dicer results in more Dicer cleaved dsRNA products, that Dicer cleavage reaction occurs more quickly compared to the processing of the same quantity of a reference dsRNA or dsRNA-containing molecule in an in vivo or in vitro assay of this disclosure, or that Dicer cleavage is directed to cleave at a specific, preferred site within a dsRNA and/or generate higher prevalence of a preferred population of cleavage products (e.g., by inclusion of DNA residues as described herein). In one embodiment, enhanced or increased Dicer cleavage of a dsRNA molecule is above the level of that observed with an appropriate reference dsRNA molecule. In another embodiment, enhanced or increased Dicer cleavage of a dsRNA molecule is above the level of that observed with an inactive or attenuated molecule.

As used herein “reduce” is meant to alter negatively by at least 5% compared to a reference in an assay. An alteration may be by 5, 10, 15, 20, 25, 30, 35, 40, 45, 50, 55, 60, 65, 70, 75, 80, 85, 90, 95, 99 or 100% compared to a reference in an assay. By “reduce expression,” it is meant that the expression of the gene, or level of RNA molecules or equivalent RNA molecules encoding one or more proteins or protein subunits, or level or activity of one or more proteins or protein subunits encoded by a target gene, is reduced below that observed in the absence of the nucleic acid molecules (e.g., dsRNA molecule or dsRNA-containing molecule) in an in vivo or in vitro assay of this disclosure. In one embodiment, inhibition, down-regulation or reduction with a dsRNA molecule is below that level observed in the presence of an inactive or attenuated molecule. In another embodiment, inhibition, down-regulation, or reduction with dsRNA molecules is below that level observed in the presence of, e.g., a dsRNA molecule with scrambled sequence or with mismatches. In another embodiment, inhibition, down-regulation, or reduction of gene expression with a nucleic acid molecule of the instant disclosure is greater in the presence of the nucleic acid molecule than in its absence.

As used herein,“cell” is meant to include both prokaryotic (e.g., bacterial) and eukaryotic (e.g., mammalian or plant) cells. Cells may be of somatic or germ line origin, may be totipotent or pluripotent, and may be dividing or non-dividing. Cells can also be derived from or can comprise a gamete or an embryo, a stem cell, or a fully differentiated cell. Thus, the term “cell” is meant to retain its usual biological meaning and can be present in any organism such as, for example, a bird, a plant, and a mammal, including, for example, a human, a cow, a sheep, an ape, a monkey, a pig, a dog, and a cat. Within certain aspects, the term “cell” refers specifically to mammalian cells, such as human cells, that contain one or more isolated nucleic acid molecules of the present disclosure. In particular aspects, a cell processes dsRNAs or dsRNA-containing molecules resulting in RNA intereference of target nucleic acids, and contains proteins and protein complexes required for RNAi, e.g., Dicer and RISC.

As used herein,“animal” is meant a multicellular, eukaryotic organism, including a mammal, particularly a human. The methods of the invention in general comprise administration of an effective amount of the molecules herein, such as an molecule of the structures of formulae herein, to a subject (e.g., animal, human) in need thereof, including a mammal, particularly a human. Such treatment will be suitably administered to subjects, particularly humans, suffering from, having, susceptible to, or at risk for a disease, or a symptom thereof.

By “pharmaceutically acceptable carrier” is meant, a composition or formulation that allows for the effective distribution of the nucleic acid molecules of the instant disclosure in the physical location most suitable for their desired activity.

The present invention is directed to isolated nucleic acid molecules and compositions comprising such molecules, which comprise both a stem-containing and -dependent aptamer and a double stranded RNA (“dsRNA”) duplex, and methods for preparing them, that are capable of reducing the expression of target genes in eukaryotic cells into which they are introduced. One of the strands of the Dicer cleavage susceptible region, i.e., which serves as the antisense strand of the molecule, contains a nucleotide sequence that has a length that ranges from about 15 to about 22 nucleotides that can direct the destruction of the target RNA (i.e., RNA transcribed from the target gene). The aptamer region of the molecule, may be chemically modified; however, whether chemically modified or not, it does not serve as a substrate for Dicer cleavage.

The nucleic acid molecules according to the invention, which contain a Dicer substrate can enhance the following attributes of such molecules relative to Dicer substrates lacking an aptamer region: in vitro efficacy (e.g., potency and duration of effect), in vivo efficacy (e.g., potency, duration of effect, pharmacokinetics, pharmacodynamics, intracellular uptake, reduced toxicity) due to the additional function which nucleic acid molecules of the invention possess, i.e., the ability to specifically bind a given receptor. In certain embodiments, the nucleic acid molecule of the instant invention provides a binding site (e.g., a cell surface receptor binding site) for a native or exogenously introduced moiety capable of binding to the nucleic acid molecule of the invention aptamer(e.g., the aptamer region can be designed to provide a sequence-specific recognition domain for a probe, marker, etc.).

As used herein, the term “pharmacokinetics” refers to the process by which a drug is absorbed, distributed, metabolized, and eliminated by the body. In certain embodiments of the instant invention, enhanced pharmacokinetics of a nucleic acid molecule containing a dsRNA and a receptor-binding region relative to an appropriate control Dicer substrate refers to increased absorption and/or distribution of such an molecule, and/or slowed metabolism and/or elimination of such a nucleic acid molecule containing a dsRNA and a receptor-binding region from a subject administered such an molecule.

As used herein, the term “pharmacodynamics” refers to the action or effect of a drug on a living organism. In certain embodiments of the instant invention, enhanced pharmacodynamics of a nucleic acid molecule containing a dsRNA and a receptor-binding region relative to an appropriate control Dicer substrate refers to an increased (e.g., more potent or more prolonged) action or effect of a nucleic acid molecule containing a dsRNA and a receptor-binding region upon a subject administered such molecule, relative to an appropriate control Dicer substrate.

As used herein, the term “stabilization” refers to a state of enhanced persistence of an molecule in a selected environment (e.g., in a cell or organism). In certain embodiments, the dsRNA-containing aptamers of the instant invention exhibit enhanced stability relative to appropriate control dsRNAs or control Dicer substrates. Such enhanced stability can be achieved via enhanced resistance of such molecules to degrading enzymes (e.g., nucleases) or other molecules.

### Preparation of Nucleic Acid Molecules According to the Invention

### Dicer Substrate Aptamers

The invention encompasses nucleic acid molecules containing a double-stranded region and a receptor binding region (“Dicer substrate aptamers”). The nucleic acid molecules can be formed by one or two polynucleotide strands. In various embodiments, the polynucleotide strand is 53-142 nucleotides in length and the 5′ terminus and 3′ terminus form a double-stranded region of at least 21-25 base pairs. The double-stranded region comprises at least 19 nucleotides complementary to a target RNA, (on the antisense strand). In other embodiments, the nucleic acid molecule has two polynucleotide strands, each 33-121 nucleotides in length, and the 5′ terminus of the polynucleotide strand containing the sense strand and the 3′ terminus of the polynucleotide strand containing the antisense strand form a double-stranded region of at least 21-25 base pairs, which contains at least 19 nucleotides complementary to a target RNA. The double-stranded region comprises ribonucleotides. The region external to the double-stranded region is a receptor binding region that specifically binds a receptor (with an affinity of at least 100 μm). This region contains an internally base-paired region comprising 4, 5, 6, 7, 8, 9, 10, 11, 12 consecutive base pairs and a single-stranded region forming a hairpin comprising 5, 6, 7, 8, 9, 10, 11, 12, 13, 14, 15, 16, 17, 18, 19, 20 consecutive non-base paired nucleotides, wherein the receptor binding affinity is dependent upon the presence of the hairpin in the nucleic acid. Dicer cleavage of the nucleic acid molecule in the double-stranded region reduces target gene expression in a mammalian cell, and reduces the ability of the nucleic acid molecule to bind selectively to the receptor.

Dicer Substrate siRNA (DsiRNA)

It has been found that longer dsRNA species of from 25 to about 30 nucleotides (DsiRNAs), and especially from 25 to 30 nucleotides yield unexpectedly effective results on RNA inhibition in terms of potency and duration of action, as compared to 19-23mer siRNA molecules. Without wishing to be bound by the underlying theory of the dsRNA processing mechanism, it is thought that the longer dsRNA species serve as a substrate for the Dicer enzyme in the cytoplasm of a cell. In addition to cleaving the dsRNA of the invention into shorter segments, Dicer is thought to facilitate the incorporation of a single-stranded cleavage product derived from the cleaved dsRNA into the RISC complex that is responsible for the destruction of the cytoplasmic RNA of or derived from the target gene. Prior studies (Rossi et al., U.S. Patent Application No. 2007/0265220) have shown that the cleavability of a dsRNA species (specifically, a DsiRNA molecule) by Dicer corresponds with increased potency and duration of action of the dsRNA species.

A schematic of the substrates and products of Dicer substrate processing is presented (e.g., in FIG. 1). Dicer enzyme processes a Dicer substrate molecule, resulting in cleavage of the Dicer substrate at a position 19-23 nucleotides removed from a Dicer PAZ domain-associated 3′ overhang sequence of the antisense strand of the Dicer substrate molecule. This Dicer cleavage event results in excision of those duplexed nucleic acids previously located at the 3′ end of the passenger (sense) strand and 5′ end of the guide (antisense) strand. (Cleavage of the Dicer substrate shown in FIG. 1 typically yields a 19 mer duplex with 2-base overhangs at each end.) As presently modeled in FIG. 1, this Dicer cleavage event generates a 21-23 nucleotide guide (antisense) strand capable of directing sequence-specific inhibition of target mRNA as a RISC component.

Design of molecules according to the invention, including DsiRNAs, can optionally involve use of predictive scoring algorithms that perform in silico assessments of the projected activity/efficacy of a number of possible Dicer substrate molecules spanning a region of sequence. Information regarding the design of such scoring algorithms can be found, e.g., in Gong et al. (BMC Bioinformatics 2006, 7:516), though a more recent “v3” algorithm represents a theoretically improved algorithm relative to siRNA scoring algorithms previously available in the art. (The “v3” scoring algorithm is a machine learning algorithm that is not reliant upon any biases in human sequence. In addition, the “v3” algorithm derives from a data set that is approximately three-fold larger than that from which an older “v2” algorithm such as that described in Gong et al. derives.)

The first and second oligonucleotides of the Dicer substrate region of the nucleic acid molecules of the instant invention are not required to be completely complementary. In fact, in one embodiment, the 3′-terminus of the sense strand contains one or more mismatches. In one aspect, about two mismatches are incorporated at the 3′ terminus of the sense strand. In another embodiment, the Dicer substrate of the invention is a double stranded RNA molecule containing two RNA oligonucleotides each of which is an identical number of nucleotides in the range of 27-35 nucleotides in length and, when annealed to each other, have blunt ends and a two nucleotide mismatch on the 3′-terminus of the sense strand (the 5′-terminus of the antisense strand). The use of mismatches or decreased thermodynamic stability (specifically at the 3′-sense/5′-antisense position) has been proposed to facilitate or favor entry of the antisense strand into RISC (Schwarz et al., 2003; Khvorova et al., 2003), presumably by affecting some rate-limiting unwinding steps that occur with entry of the siRNA into RISC. Thus, terminal base composition has been included in design algorithms for selecting active 21mer siRNA duplexes (Ui-Tei et al., 2004; Reynolds et al., 2004). With Dicer cleavage of the dsRNA region of this embodiment, the small end-terminal sequence which contains the mismatches will either be left unpaired with the antisense strand (become part of a 3′-overhang) or be cleaved entirely off the final 21-mer siRNA. These specific forms of “mismatches”, therefore, do not persist as mismatches in the final RNA component of RISC. The finding that base mismatches or destabilization of segments at the 3′-end of the sense strand of Dicer substrate improved the potency of synthetic duplexes in RNAi, presumably by facilitating processing by Dicer, was a surprising finding of past works describing the design and use of 25-30mer dsRNAs (also termed “DsiRNAs” herein; Rossi et al., U.S. Patent Application Nos. 2005/0277610, 2005/0244858 and 2007/0265220).

### Dicer Substrate Aptamers Design/Synthesis

Nucleic acid molecules of the invention can be made by providing one or two polynucleotides that have a sequence encoding a dsRNA directed to reducing the expression of a target gene and a randomized sequence. The nucleic acid molecules containing sequences for the dsRNA and the randomized sequence are selected for a desired function (e.g., receptor binding, Dicer cleavage) using a selection method (e.g., SELEX). In various embodiments, a polynucleotide strand 53-142 nucleotides in length is synthesized with the dsRNA sequences at the 5′ terminus and 3′ terminus, flanking the region having the randomized sequence, which is selected for a desired property (e.g., receptor binding, Dicer cleavage). Thus, the dsRNA formed by the 5′ terminus and the 3′ terminus of the polynucleotide strand is 21-25 base-pairs in length. The polynucleotide is selected for the desired property under conditions that the double-stranded region at least forms. Conditions for hybridization of nucleic acids, and thus formation of the double-stranded region, are known in the art and described herein. Nucleic acid molecules of the invention formed by a polynucleotide strand are isolated and identified in this manner In other embodiments, two polynucleotide strands are synthesized. In these embodiments, the first polynucleotide strand is 33-121 nucleotides in length and has a sequence encoding the sense strand of a dsRNA starting at the 5′ terminus The dsRNA sense sequence is followed by a region of randomized sequence continuing to the 3′ terminus of the first strand. The second polynucleotide strand is 33-121 nucleotides in length and has a sequence encoding a sequence complementary to the sense strand sequence (i.e., an antisense strand sequence) at the 3′ terminus The 5′ terminus of the first polynucleotide strand and the 3′ terminus of the second polynucleotide strand are hybridized under conditions known in the art and described herein to form a double-stranded region of at least 21-25 base pairs. The nucleic acid molecule formed by the two polynucleotide strands is selected for a desired property (e.g., receptor binding, Dicer cleavage) under conditions that the double-stranded region at least forms. Nucleic acid molecules of the invention formed by two polynucleotide strands are isolated and identified in this manner.

Additionally, nucleic acid molecules of the invention can be made by providing one or two polynucleotides that have a sequence encoding a dsRNA directed to reducing the expression of a target gene covalently attached to a nucleic acid aptamer. These Dicer substrate aptamers typically contain at least one region primarily comprising ribonucleotides (optionally including modified ribonucleotides) that form a Dicer substrate siRNA (“DsiRNA”) molecule. This Dicer substrate region is covalently attached to a second region comprising a nucleic acid aptamer, which confers one or more beneficial properties (such as, for example, increased efficacy, e.g., increased potency and/or duration of Dicer substrate activity, function as a recognition domain or means of targeting a chimeric dsRNA to a specific location, for example, when administered to cells in culture or to a subject, functioning as an extended region for improved attachment of functional groups, payloads, detection/detectable moieties, functioning as an extended region that allows for more desirable modifications and/or improved spacing of such modifications, etc.). This second region comprising a nucleic acid aptamer may also include modified or synthetic nucleotides and/or modified or synthetic deoxyribonucleotides. In certain embodiments, a chimeric Dicer substrate/ nucleic acid aptamer of the invention comprises at least one region (“aptamer region”), located 3′ of the projected Dicer cleavage site of the first strand and 5′ of the projected Dicer cleavage site of the second strand, having a secondary and/or tertiary structure. The structure of the aptamer region may be selected for a functional process by SELEX or another in vitro selection process. In some embodiments, the first and second strands of the Dicer substrate share the same backbone with the stem dependent aptamer (e.g., the 3′ end of the first strand of the Dicer substrate is connected by a 3′-5′ phosphodiester linkage to the 5′ end of the nucleic acid aptamer and the 3′ end of the nucleic acid aptamer is connected by a 3′-5′ phosphodiester linkage to the 5′ end of the second strand of the Dicer substrate—see, e.g., FIG. 2). In other embodiments, the first and second strands of the Dicer substrate share a backbone with two polynucleotides which form a stem dependent aptamer (e.g., the 3′ end of the first strand of the Dicer substrate is connected by a 3′-5′ phosphodiester linkage to the 5′ end of the nucleic acid aptamer, the 3′ end of the nucleic acid aptamer is connected by a 3′-5′ phosphodiester linkage to the 5′ end of the second strand of the Dicer substrate, and the two strands form a duplex in the Dicer substrate region and, although discontinuous, adopt appropriate secondary and/or tertiary structure in the aptamer region).

### Systematic Evolution of Ligands by Exponential Enrichment (SELEX)

A method for the in vitro evolution of nucleic acid molecules with highly specific binding to target molecules has been developed. This method, Systematic Evolution of Ligands by EXponential enrichment, termed SELEX, is described in U.S. patent application Ser. No. 07/536, 428, entitled “Systematic Evolution of Ligands by Exponential Enrichment, ” now abandoned, U.S. patent application Ser. No. 07/714,131, filed Jun. 10, 1991, entitled “Nucleic Acid Ligands,” now U.S. Pat. No. 5, 475,096, U.S. patent application Ser. No. 07/931,473, filed Aug. 17, 1992, entitled “Methods for Identifying Nucleic Acid Ligands,” now U.S. Pat. No. 5,270,163 (see also WO 91/19813), each of which is herein specifically incorporated by reference. Each of these applications, collectively referred to herein as the SELEX Patent Applications, describes a method for making a nucleic acid ligand to any desired target molecule. Additionally, SELEX may be performed against whole cells rather than purified cell surface markers (Hicke et al. Biol Chem. 2001 Dec. 28; 276(52):48644-54, Daniels et al., Anal Biochem. 2002 Jun. 15; 305(2):214-26, and Daniels et al. Proc Natl Acad Sci USA. 2003 Dec. 23; 100(26):15416-212003, which are herein incorporated by reference).

The SELEX method involves selection from a mixture of candidate oligonucleotides and step-wise iterations of binding, partitioning and amplification, using the same general selection scheme, to achieve virtually any desired criterion of binding affinity and selectivity. Starting from a mixture of nucleic acids, preferably comprising a segment of randomized sequence, the SELEX method includes steps of contacting the mixture with the target under conditions favorable for binding, partitioning unbound nucleic acids from those nucleic acids which have bound specifically to target molecules, dissociating the nucleic acid-target complexes, amplifying the nucleic acids dissociated from the nucleic acid-target complexes to yield a ligand-enriched mixture of nucleic acids, then reiterating the steps of binding, partitioning, dissociating and amplifying through as many cycles as desired to yield highly specific, high affinity nucleic acid ligands to the target molecule.

The basic SELEX method has been modified to achieve a number of specific objectives. For example, U.S. patent application Ser. No. 07/960, 093, filed Oct. 14, 1992, entitled “Method for Selecting Nucleic Acids on the Basis of Structure,” describes the use of SELEX in conjunction with gel electrophoresis to select nucleic acid molecules with specific structural characteristics, such as bent DNA. U.S. patent application Ser. No. 08/123,935, filed Sep. 17, 1993, entitled “Photoselection of Nucleic Acid Ligands” describes a SELEX based method for selecting nucleic acid ligands containing photoreactive groups capable of binding and/or photocrosslinking to and/or photoinactivating a target molecule. U.S. patent application Ser. No. 08/134,028, filed Oct. 7, 1993, entitled “High-Affinity Nucleic Acid Ligands That Discriminate Between Theophylline and Caffeine,” describes a method for identifying highly specific nucleic acid ligands able to discriminate between closely related molecules, termed “Counter-SELEX.” U.S. patent application Ser. No. 08/143,564, filed Oct. 25, 1993, entitled “Systematic Evolution of Ligands by EXponential Enrichment: Solution SELEX,” now abandoned (see U.S. Pat. No. 5,567,588), describes a SELEX-based method which achieves highly efficient partitioning between oligonucleotides having high and low affinity for a target molecule. U.S. patent application Ser. No. 07/964,624, filed Oct. 21, 1992, entitled “Nucleic Acid Ligands to HIV-RT and HIV-1 Rev,” now issued as U.S. Pat. No. 5,496,938, describes methods for obtaining improved nucleic acid ligands after SELEX has been performed. U.S. patent application Ser. No. 08/400,440, filed Mar. 8, 1995, entitled “Systematic Evolution of Ligands by EXponential Enrichment: Chemi-SELEX,” now issued as U.S. Pat. No. 5,705,337, describes methods for covalently linking a ligand to its target.

The SELEX method encompasses the identification of high-affinity nucleic acid ligands containing modified nucleotides conferring improved characteristics on the ligand, such as improved in vivo stability or improved delivery characteristics. Examples of such modifications include chemical substitutions at the ribose and/or phosphate and/or base positions. SELEX-identified nucleic acid ligands containing modified nucleotides are described in U.S. patent application Ser. No. 08/117,991, filed Sep. 8, 1993, entitled “High Affinity Nucleic Acid Ligands Containing Modified Nucleotides,” now abandoned (see, U.S. Pat. No. 5,660,985), that describes oligonucleotides containing nucleotide derivatives chemically modified at the 5- and 2′-positions of pyrimidines. U.S. patent application Ser. No. 08/134,028, supra, describes highly specific nucleic acid ligands containing one or more nucleotides modified with 2′-amino (2′-NH ₂), 2′-fluoro (2′-F), and/or 2′-O-methyl (2′-O-Me). U.S. patent application Ser. No. 08/264,029, filed Jun. 22, 1994, entitled “Novel Method of Preparation of Known and Novel 2′-Modified Nucleosides by Intramolecular Nucleophilic Displacement,” describes oligonucleotides containing various 2′-modified pyrimidines.

The SELEX method encompasses combining selected oligonucleotides with other selected oligonucleotides and non-oligonucleotide functional units as described in U.S. patent application Ser. No. 08/284,063, filed Aug. 2, 1994, entitled “Systematic Evolution of Ligands by Exponential Enrichment Chimeric SELEX”, now U.S. Pat. No. 5,637,459, and U.S. patent application Ser. No. 08/234,997, filed Apr. 28, 1994, entitled “Systematic Evolution of Ligands by Exponential Enrichment: Blended SELEX,” now U.S. Pat. No. 5,683,867, respectively. These applications allow the combination of the broad array of shapes and other properties, and the efficient amplification and replication properties, of oligonucleotides with the desirable properties of other molecules. Each of the above described patent applications which describe modifications of the basic SELEX procedure are specifically incorporated by reference herein in their entirety.

Methods of the invention for making a Dicer substrate aptamer involve contacting a Dicer substrate aptamer with a receptor, isolating the Dicer substrate aptamer bound to the receptor, and contacting the isolated Dicer substrate aptamer with Dicer enzyme. As is apparent to one of skill in the art, the methods of the invention can be adapted to select, identify, and/or isolate Dicer substrate aptamers using systematic evolution of ligands by exponential enrichment (SELEX). A Dicer substrate aptamer has the property that Dicer cleavage of the nucleic acid molecule in the double-stranded region reduces the ability of the aptamer to bind selectively to the receptor. A Dicer substrate aptamer is capable of being processed by Dicer, including in the presence of the receptor (i.e., receptor binding does not interfere with the ability of Dicer to cleave the Dicer substrate aptamer). Methods described herein or known in the art, including biological and biochemical assays, high-throughput methods, polymerase chain reaction (PCR), nucleic acid sequencing may be employed in systematic evolution of ligands by exponential enrichment (SELEX) to make the Dicer substrate aptamers of the invention. Additionally, one or more SELEX selection procedures may be used to make the Dicer substrate aptamers of the invention For example, in one embodiment of the invention, an additional step analyzes the products of the Dicer cleavage (i.e., the receptor binding region or aptamer) for receptor binding of a putative Dicer substrate aptamer. Based on this assay, a Dicer substrate aptamer generates Dicer cleavage products that do not substantially bind the receptor.

### Binding Assay

Any assay known in the art may be used for measuring binding, including measurements for association rate (‘on rate’, vₒₙ), dissociation rate (‘off rate’, v_{off}), Such assays without limitation include standard biochemical or physical chemicstry methods, e.g., surface plasmon resonance (SPR) on BIACORE (BIAcore AB, Uppsala, Sweden). A receptor or fragment thereof is immobilized on the dextran surface of the SPR crystal. Through a microflow system, a solution with the Dicer substrate aptamer is injected over the immobilized receptor. As the Dicer substrate aptamer binds the receptor, an increase in SPR signal (expressed in response units, RU) is observed. After a desired association time, a solution without the Dicer substrate aptamer (usually the buffer) is injected that dissociates the bound complex between the receptor and the Dicer substrate aptamer. As the Dicer substrate aptamer dissociates from the receptor, a decrease in SPR signal (expressed in response units, RU) is observed. Without being bound to a particular theory, the SPR signal is explained by the electromagnetic ‘coupling’ of the incident light with the surface plasmon of the gold layer. This plasmon is influenced by the layer just a few nanometer across the gold-solution interface i.e. the receptor and possibly the Dicer substrate aptamer. Binding makes the reflection angle change. From these observations, association (‘on rate’, vₒₙ) and dissociation rates (‘off rate’, v_{off}), and the binding constant can be calculated.

A binding assay may also be performed using whole cells. Hicke et al. Biol Chem. 2001 Dec. 28; 276(52):48644-54, Daniels et al., Anal Biochem. 2002 Jun. 15; 305(2):214-26, and Daniels et al. Proc Natl Acad Sci USA. 2003 Dec. 23; 100(26):15416-212003, which are herein incorporated by reference describe binding assays on whole cells.

Binding affinities for Dicer substrate aptamers (i.e., not Dicer cleaved) is at least 100 μm, preferably 1-100 μm, more preferably 1-100 nm, and even more preferably 1-100 μm. Table 2 lists exemplary affinities for nucleic acid aptamers isolated by SELEX (adapted from Table 17.1, The Aptamer Handbook WILEY-VCH 2006).

TABLE 2
Aptamers to Adhesion molecules, receptors, and other cell surface
proteins

| Protein | Affinity | | Reference |
|---|---|---|---|
| L-Selectin | 60 | pmol/L | Watson et al, 2000 |
| P-Selectin | 29 | pmol/L | Jenison et al, 1998 |
| LFA-1 (CDI 8) | 500 | nmol/L | Blind et al., 1999 |
| | −1 | μmol/L | |
| PSMA | 2 | nmol/L | Lupold et al. 2002 |
| HER3 | 45 | nmol/L | Chen et al., 2003 |
| CD4 | ND | | Kraus et al.., 1998 |
| CTLA-4 | ~50 | nmol/L | Santulli-Marotto et al., |
| | | | 2003 |
| TenascimC | 5 | nmol/L | Hicke et al., 2001 |

| | | |
|---|---|---|
| Pigpen | ND | Blank et al., 2001 |

| | | | |
|---|---|---|---|
| Trypanosma brucei | 160 | pmol/L | Lorger et al., 2003 |
| VSD protein | | | |
| Trypanosma brucei | 60 | nmoI/L | Homann et al., 1999 |
| (flagellar pocket protein) | | | |
| Tryoanosma cruzi | ~100 | nmol/L | Ulrich et al., 2002 |
| cell surface receptor | | | |

### Dicer Assay

A cell-free reaction to assay Dicer cleavage may be performed in vitro involving contacting the Dicer substrate aptamer with a purified Dicer protein. Dicer cleavage is determined as follows (e.g., see Collingwood et al., Oligonucleotides 18:187-200 (2008)). In a Dicer cleavage assay, Dicer substrate aptamers or RNA duplexes (100 μmol) are incubated in 20 μL of 20 mM Tris pH 8.0, 200 mM NaCl, 2.5 mM MgCl₂ with or without 1 unit of recombinant human Dicer (Stratagene, La Jolla, Calif.) at 37° C. for 18-24 hours. Samples are desalted using a Performa SR 96-well plate (Edge Biosystems, Gaithersburg, Md.). Electrospray-ionization liquid chromatography mass spectroscopy (ESI-LCMS) of duplex RNAs pre- and post-treatment with Dicer is done using an Oligo HTCS system (Novatia, Princeton, N.J.; Hail et al., 2004), which consists of a ThermoFinnigan TSQ7000, Xcalibur data system, ProMass data processing software and Paradigm MS4 HPLC (Michrom BioResources, Auburn, Calif.). In this assay, Dicer cleavage occurs where at least 5%, 10%, 20%, 30%, 40%, 50%, 60%, 70%, 80%, 90%, 95%, or even 100% of the Dicer substrate aptamer (e.g., as described herein) is cleaved to a shorter dsRNA (e.g., 19-23 bp dsRNA, preferably, 21-23 bp dsRNA).

A cell-free reaction to assay Dicer cleavage may be combined with either a subsequent cell-free measurement of aptamer-binding, or nuclease degradation assay using electrophoresis to separate and visualize whole Dicer substrate-aptamers from processed siRNA and free aptamer to confirm Dicer cleavage, and then to measure the extent of degradation in the presence of nucleases (using cell lysates or plasma as the general nuclease source) could do this. Dicer assays may be performed intracellularly.

### Aptamers

An “aptamer” may be a nucleic acid molecule, such as RNA or DNA that is capable of binding to a specific molecule with high affinity and/or specificity (Ellington et al., Nature 346, 818-22 (1990); and Tuerk et al., Science 249, 505-10 (1990)). Exemplary ligands that bind to an aptamer include, without limitation, small molecules, such as drugs, metabolites, intermediates, cofactors, transition state analogs, ions, metals, nucleic acids, and toxins. Aptamers may also bind natural and synthetic polymers, including proteins, peptides, nucleic acids, polysaccharides, glycoproteins, hormones, receptors and cell surfaces such as cell walls and cell membranes. An aptamer will most typically have been obtained by in vitro selection for binding of a target molecule. However, in vivo selection of an aptamer is also possible. Aptamers have specific binding regions which are capable of forming complexes with an intended target molecule in an environment wherein other substances in the same environment are not complexed to the nucleic acid. An aptamer comprises at least one loop. The secondary and/or tertiary structure of the aptamer may contribute to the selective binding of an aptamer and target ligand (e.g., a ligand that is not a nucleic acid). A nucleic acid aptamer in the invention forms structures which are not cleaved by Dicer enzyme. For example, RNA aptamers are highly chemically modified and not cleaved by Dicer enzyme.

The specificity of the binding is defined in terms of the comparative dissociation constants (Kd) of the aptamer for its ligand as compared to the dissociation constant of the aptamer for other materials in the environment or unrelated molecules in general. A ligand is one which binds to the aptamer with greater affinity than to unrelated material. Typically, the dissociation constant (Kd) for the aptamer with respect to its ligand will be at least about 10-fold less than the Kd for the aptamer with unrelated material or accompanying material in the environment. Even more preferably, the Kd will be at least about 50-fold less, more preferably at least about 100-fold less, and most preferably at least about 200-fold less. An aptamer will typically be between about 10 and about 400 nucleotides in length. More commonly, an aptamer will be between about 30 and about 100 nucleotides in length, more preferably 20-100 nucleotides, and most preferably 25-50 nucleotides.

Aptamers are readily made that bind to a wide variety of molecules. Each of these molecules can be used as a modulator of gene expression using the methods of the invention. For example, organic molecules, nucleotides, amino acids, polypeptides, target features on cell surfaces, ions, metals, salts, saccharides, have all been shown to be suitable for isolating aptamers that can specifically bind to the respective ligand. For instance, organic dyes such as Hoechst 33258 have been successfully used as target ligands in vitro aptamer selections (Werstuck and Green, Science 282:296-298 (1998)). Other small organic molecules like dopamine, theophylline, sulforhodamine B, and cellobiose have also been used as ligands in the isolation of aptamers. Aptamers have also been isolated for antibiotics such as kanamycin A, lividomycin, tobramycin, neomycin B, viomycin, chloramphenicol and streptomycin. For a review of aptamers that recognize small molecules, see Famulok, Science 9:324-9 (1999).

In certain embodiments, the receptor of a nucleic acid molecule of the invention is a cell surface molecule. Cell surface receptors that are internalized are preferred. Receptors include without limitation proteins, glycoproteins, channels, cadherins, desmosomes, internal proteins inappropriately expressed on cell surfaces, viral or other pathogen markers expressed or displayed on the cell surfaces. For example, specific receptors include nucleolin, a human epidermal growth factor receptor 2 (HER2), CD20. The cell surface molecule preferably also exhibits in vivo persistence sufficient for achieving the desired level of inhibition of translation. The molecules also can be screened to identify those that are bioavailable after, for example, oral administration. In certain embodiments of the invention, the ligand is nontoxic. The ligand may optionally be a drug, including, for example, a steroid. However, in some of the methods of controlling gene expression, it is preferable that the ligand be pharmacologically inert. In some embodiments, the ligand is a polypeptide whose presence in the cell is indicative of a disease or pathological condition.

Aptamers are typically developed to bind particular ligands by employing known in vivo or in vitro (most typically, in vitro) selection techniques known as systematic evolution of ligands by exponential enrichment (SELEX) (Ellington et al., Nature 346, 818-22 (1990); and Tuerk et al., Science 249, 505-10 (1990)). In systematic evolution of ligands by exponential enrichment (SELEX), nucleic acid species are engineered through repeated rounds of in vitro selection to generate aptamers. Methods of making aptamers are also described in, for example, U.S. Pat. No. 5,582,981, PCT Publication No. WO 00/20040, U.S. Pat. No. 5,270,163, Lorsch and Szostak, Biochemistry, 33:973 (1994), Mannironi et al., Biochemistry 36:9726 (1997), Blind, Proc. Nat'l. Acad. Sci. USA 96:3606-3610 (1999), Huizenga and Szostak, Biochemistry, 34:656-665 (1995), PCT Publication Nos. WO 99/54506, WO 99/27133, WO 97/42317 and U.S. Pat. No. 5,756,291.

Generally, in their most basic form, in vitro selection techniques for identifying aptamers involve first preparing a large pool of DNA molecules of the desired length that contain at least some region that is randomized or mutagenized. For instance, a common oligonucleotide pool for aptamer selection might contain a region of 20-100 randomized nucleotides flanked on both ends by an about 15-25 nucleotide long region of defined sequence useful for the binding of PCR primers. In the methods of the invention, the flanking regions may comprise the strands of a dsRNA. The oligonucleotide pool is amplified using standard PCR techniques, although any means that will allow faithful, efficient amplification of selected nucleic acid sequences can be employed. The DNA pool is then in vitro transcribed to produce RNA transcripts. The RNA transcripts may then be subjected to affinity chromatography, although any protocol which will allow selection of nucleic acids based on their ability to bind specifically to another molecule (e.g., a protein or any target molecule) may be used. In the case of affinity chromatography, the transcripts are most typically passed through a column or contacted with magnetic beads or the like on which the target ligand has been immobilized. RNA molecules in the pool which bind to the ligand are retained on the column or bead, while nonbinding sequences are washed away. The RNA molecules which bind the ligand are then reverse transcribed and amplified again by PCR (usually after elution). The selected pool sequences are then put through another round of the same type of selection. Typically, the pool sequences are put through a total of about three to ten iterative rounds of the selection procedure. The cDNA is then amplified, cloned, and sequenced using standard procedures to identify the sequence of the RNA molecules which are capable of acting as aptamers for the target ligand. Once an aptamer sequence has been successfully identified, the aptamer may be further optimized by performing additional rounds of selection starting from a pool of oligonucleotides comprising the mutagenized aptamer sequence. For use in the present invention, the aptamer is preferably selected for ligand binding in the presence of salt concentrations and temperatures which mimic normal physiological conditions.

One can generally choose a suitable ligand without reference to whether an aptamer is yet available. In most cases, an aptamer can be obtained which binds the ligand of choice by someone of ordinary skill in the art. The unique nature of the in vitro selection process allows for the isolation of a suitable aptamer that binds a desired ligand despite a complete dearth of prior knowledge as to what type of structure might bind the desired ligand.

For an aptamer to be suitable for use in the present invention, the binding affinity of the aptamer for the ligand must be sufficiently strong. The aptamer preferably binds the target ligand with an affinity in the micromolar range (1-100 μM) and more preferably with an affinity in the nanomolar to picomolar range (1-100 nM affinity and 1-100 pM affinity). That is, the aptamer will selectively bind to the target molecule or cell with an affinity that is at least 10-fold greater affinity than the affinity with which the aptamer binds to a non-target molecule.

The association constant for the aptamer and associated ligand is preferably such that the ligand functions to bind to the aptamer and have the desired effect at the concentration of ligand obtained upon administration of the ligand. For in vivo use, for example, the association constant should be such that binding occurs well below the concentration of ligand that can be achieved in the serum or other tissue. Preferably, the required ligand concentration for in vivo use is also below that which could have undesired effects on the organism.

### RNA Processing

siRNA

The process of siRNA-mediated RNAi is triggered by the presence of long, dsRNA molecules in a cell. In the invention, the receptor-binding nucleic acid molecules contain a Dicer substrate siRNA (“DsiRNAs”). During the initiation step of RNAi, these dsRNA molecules are cleaved into 21-23 nucleotide (nt) small-interfering RNA duplexes (siRNAs) by Dicer, a conserved family of enzymes containing two RNase III-like domains (Bernstein et al. 2001; Elbashir et al. 2001). The siRNAs are characterized by a 19-21 base pair duplex region and 2 nucleotide 3′ overhangs on each strand. During the effector step of RNAi, the siRNAs become incorporated into a multimeric protein complex called RNA-induced silencing complex (RISC), where they serve as guides to select fully complementary mRNA substrates for degradation. Degradation is initiated by endonucleolytic cleavage of the mRNA within the region complementary to the siRNA. More precisely, the mRNA is cleaved at a position 10 nucleotides from the 5′ end of the guiding siRNA (Elbashir et al. 2001 Genes & Dev. 15: 188-200; Nykanen et al. 2001 Cell 107: 309-321; Martinez et al. 2002 Cell 110: 563-574). An endonuclease responsible for this cleavage was identified as Argonaute2 (Ago2; Liu et al. Science, 305: 1437-41).

### RNase H

RNase H is a ribonuclease that cleaves the 3′-O—P bond of RNA in a DNA/RNA duplex to produce 3′-hydroxyl and 5′-phosphate terminated products. RNase H is a non-specific endonuclease and catalyzes cleavage of RNA via a hydrolytic mechanism, aided by an enzyme-bound divalent metal ion. Members of the RNase H family are found in nearly all organisms, from archaea and prokaryotes to eukaryotes. During DNA replication, RNase H is believed to cut the RNA primers responsible for priming generation of Okazaki fragments; however, the RNase H enzyme may be more generally employed to cleave any DNA:RNA hybrid sequence of sufficient length (e.g., typically DNA:RNA hybrid sequences of 4 or more base pairs in length in mammals).

### Modification of Dicer Substrate Aptamers

One major factor that inhibits the effect of nucleic acid molecules is the degradation of nucleic acid (e.g., Dicer substrate aptamers, DsiRNAs) by nucleases. A 3′-exonuclease is the primary nuclease activity present in serum and modification of the 3′-ends of antisense DNA oligonucleotides is crucial to prevent degradation (Eder et al., 1991). An RNase-T family nuclease has been identified called ERI-1 which has 3′ to 5′ exonuclease activity that is involved in regulation and degradation of siRNAs (Kennedy et al., 2004; Hong et al., 2005). This gene is also known as Thex1 (NM_{—}02067) in mice or THEX1 (NM_{—}153332) in humans and is involved in degradation of histone mRNA; it also mediates degradation of 3′-overhangs in siRNAs, but does not degrade duplex RNA (Yang et al., 2006). It is therefore reasonable to expect that 3′-end-stabilization of dsRNAs, including the Dicer substrates of the instant invention, will improve stability.

XRN1 (NM_{—}019001) is a 5′ to 3′ exonuclease that resides in P-bodies and has been implicated in degradation of mRNA targeted by miRNA (Rehwinkel et al., 2005) and may also be responsible for completing degradation initiated by internal cleavage as directed by a siRNA. XRN2 (NM _{—}012255) is a distinct 5′ to 3′ exonuclease that is involved in nuclear RNA processing. Although not currently implicated in degradation or processing of siRNAs and miRNAs, these both are known nucleases that can degrade RNAs and may also be important to consider.

RNase A is a major endonuclease activity in mammals that degrades RNAs. It is specific for ssRNA and cleaves at the 3′-end of pyrimidine bases. SiRNA degradation products consistent with RNase A cleavage can be detected by mass spectrometry after incubation in serum (Turner et al., 2007). The 3′-overhangs enhance the susceptibility of siRNAs to RNase degradation. Depletion of RNase A from serum reduces degradation of siRNAs; this degradation does show some sequence preference and is worse for sequences having poly A/U sequence on the ends (Haupenthal et al., 2006). This suggests the possibility that lower stability regions of the duplex may “breathe” and offer transient single-stranded species available for degradation by RNase A. RNase A inhibitors can be added to serum and improve siRNA longevity and potency (Haupenthal et al., 2007).

Nucleic acids of the invention suitable for systemic use in vivo typically have high levels of chemical modification. Such chemical modifications may contribute to the binding interactions with a receptor. Because of the chemical modifications, the nucleic acid molecules of the invention are highly nuclease resistant. In systemic delivery methods, this nuclease resistance results in an increase in half-life in serum. Within a cell, nuclease resistance reduces off target effects caused by the activity of nucleases (e.g., Dicer) on the nucleic acid molecules of the invention. However, accumulation of chemically modified nucleic acid molecules of the invention has the potential to cause detrimental effects due to their ability to bind proteins and should be minimized

In dsRNA regions of the nucleic acids of the invention, phosphorothioate or boranophosphate modifications directly stabilize the internucleoside phosphate linkage. Boranophosphate modified RNAs are highly nuclease resistant, potent as silencing molecules, and are relatively non-toxic. Boranophosphate modified RNAs cannot be manufactured using standard chemical synthesis methods and instead are made by in vitro transcription (IVT) (Hall et al., 2004 and Hall et al., 2006). Phosphorothioate (PS) modifications can be readily placed in an RNA duplex at any desired position and can be made using standard chemical synthesis methods, though the ability to use such modifications within an RNA duplex that retains RNA silencing activity can be limited. It is noted, however, that the PS modification shows dose-dependent toxicity, so most investigators have recommended limited incorporation in siRNAs, historically favoring the 3′-ends where protection from nucleases is most important (Harborth et al., 2003; Chiu and Rana, 2003; Braasch et al., 2003; Amarzguioui et al., 2003). More extensive PS modification can be compatible with potent RNAi activity; however, use of sugar modifications (such as 2′-O-methyl RNA) may be superior (Choung et al., 2006).

A variety of substitutions can be placed at the 2′-position of the ribose in nucleic acids of the invention. In Dicer substrate regions these substitutions generally increases duplex stability (Tₘ) and can greatly improve nuclease resistance. 2′-O-methyl RNA is a naturally occurring modification found in mammalian ribosomal RNAs and transfer RNAs. 2′-O-methyl modification in siRNAs is known, but the precise position of modified bases within the duplex of the stem structure is important to retain potency and complete substitution of 2′-O-methyl RNA for RNA will inactivate the Dicer substrate. For example, a pattern that employs alternating 2′-O-methyl bases can have potency equivalent to unmodified RNA and is quite stable in serum (Choung et al., 2006; Czauderna et al., 2003). Nuclease resistance assays may be utilized to determine stability of a given isolated nucleic acid according to the invention, as is know in the prior art; e.g., Choung et al., 2006 and Czauderna et al., 2003).

The 2′-fluoro (2′-F) modification can be used to modify nucleic acids of the invention and is also compatible with dsRNA (e.g., siRNA and DsiRNA) function. In Dicer substrate regions it is most commonly placed at pyrimidine sites (due to remolecule cost and availability) and can be combined with 2′-O-methyl modification at purine positions; 2′-F purines are available and can also be used. Heavily modified duplexes of this kind can be potent triggers of RNAi in vitro (Allerson et al., 2005; Prakash et al., 2005; Kraynack and Baker, 2006) and can improve performance and extend duration of action when used in vivo (Morrissey et al., 2005a; Morrissey et al., 2005b). A highly potent, nuclease stable, blunt 19mer duplex containing alternative 2′-F and 2′-O-Me bases is taught by Allerson. In this design, alternating 2′-O-Me residues are positioned in an identical pattern to that employed by Czauderna, however the remaining RNA residues are converted to 2′-F modified bases. A highly potent, nuclease resistant siRNA employed by Morrissey employed a highly potent, nuclease resistant siRNA in vivo. In addition to 2′-O-Me RNA and 2′-F RNA, this duplex includes DNA, RNA, inverted abasic residues, and a 3′-terminal PS internucleoside linkage. While extensive modification has certain benefits, more limited modification of the duplex can also improve in vivo performance and is both simpler and less costly to manufacture. Soutschek et al. (2004) employed a duplex in vivo and was mostly RNA with two 2′-O-Me RNA bases and limited 3′-terminal PS internucleoside linkages.

Locked nucleic acids (LNAs) are a different class of 2′-modification that can be used to stabilize nucleic acids of the invention and dsRNAs (e.g., siRNA and DsiRNA). In Dicer substrate RNAs, patterns of LNA incorporation that retain potency are more restricted than 2′-O-methyl or 2′-F bases, so limited modification is preferred (Braasch et al., 2003; Grunweller et al., 2003; Elmen et al., 2005). Even with limited incorporation, the use of LNA modifications can improve dsRNA performance in vivo and may also alter or improve off target effect profiles (Mook et al., 2007).

Synthetic nucleic acids introduced into cells or live animals can be recognized as “foreign” and trigger an immune response Immune stimulation constitutes a major class of off-target effects which can dramatically change experimental results and even lead to cell death. The innate immune system includes a collection of receptor molecules that specifically interact with DNA and RNA that mediate these responses, some of which are located in the cytoplasm and some of which reside in endosomes (Marques and Williams, 2005; Schlee et al., 2006). Delivery of siRNAs by cationic lipids or liposomes exposes the siRNA to both cytoplasmic and endosomal compartments, maximizing the risk for triggering a type 1 interferon (IFN) response both in vitro and in vivo (Morrissey et al., 2005b; Sioud and Sorensen, 2003; Sioud, 2005; Ma et al., 2005). RNAs transcribed within the cell are less immunogenic (Robbins et al., 2006) and synthetic RNAs that are immunogenic when delivered using lipid-based methods can evade immune stimulation when introduced unto cells by mechanical means, even in vivo (Heidel et al., 2004). However, lipid based delivery methods are convenient, effective, and widely used. Some general strategy to prevent immune responses is needed, especially for in vivo application where all cell types are present and the risk of generating an immune response is highest. Use of chemically modified RNAs may solve most or even all of these problems.

Although certain sequence motifs are clearly more immunogenic than others, it appears that the receptors of the innate immune system in general distinguish the presence or absence of certain base modifications which are more commonly found in mammalian RNAs than in prokaryotic RNAs. For example, pseudouridine, N6-methyl-A, and 2′-O-methyl modified bases are recognized as “self” and inclusion of these residues in a synthetic RNA can help evade immune detection (Kariko et al., 2005). Extensive 2′-modification of a sequence that is strongly immunostimulatory as unmodified RNA can block an immune response when administered to mice intravenously (Morrissey et al., 2005b). However, extensive modification is not needed to escape immune detection and substitution of as few as two 2′-O-methyl bases in a single strand of a siRNA duplex can be sufficient to block a type 1 IFN response both in vitro and in vivo; modified U and G bases are most effective (Judge et al., 2006). As an added benefit, selective incorporation of 2′-O-methyl bases can reduce the magnitude of off-target effects (Jackson et al., 2006). Use of 2′-O-methyl bases should therefore be considered for all dsRNAs intended for in vivo applications as a means of blocking immune responses and has the added benefit of improving nuclease stability and reducing the likelihood of off-target effects.

Although cell death can result from immune stimulation, assessing cell viability is not an adequate method to monitor induction of IFN responses. IFN responses can be present without cell death, and cell death can result from target knockdown in the absence of IFN triggering (for example, if the targeted gene is essential for cell viability). Relevant cytokines can be directly measured in culture medium and a variety of commercial kits exist which make performing such assays routine. While a large number of different immune effector molecules can be measured, testing levels of IFN-α, TNF-α, and IL-6 at 4 and 24 hours post transfection is usually sufficient for screening purposes. It is important to include a “transfection remolecule only control” as cationic lipids can trigger immune responses in certain cells in the absence of any nucleic acid cargo. Including controls for IFN pathway induction should be considered for cell culture work. It is essential to test for immune stimulation whenever administering nucleic acids in vivo, where the risk of triggering IFN responses is highest.

Modifications can be included in the nucleic acid molecules of the present invention so long as the modification does not prevent the Dicer substrate molecule from serving as a substrate for Dicer. In one embodiment, one or more modifications are made that enhance Dicer processing of the Dicer substrate molecule. In a second embodiment, one or more modifications are made that result in more effective RNAi generation. In a third embodiment, one or more modifications are made that support a greater RNAi effect. In a fourth embodiment, one or more modifications are made that result in greater potency per each Dicer substrate molecule to be delivered to the cell. Modifications can be incorporated in the 3′-terminal region, the 5′-terminal region, in both the 3′-terminal and 5′-terminal region or in some instances in various positions within the sequence. With the restrictions noted above in mind, any number and combination of modifications can be incorporated into the Dicer substrate portion of the molecule. Where multiple modifications are present, they may be the same or different. Modifications to bases, sugar moieties, the phosphate backbone, and their combinations are contemplated. Either 5′-terminus can be phosphorylated.

Examples of modifications contemplated for the phosphate backbone include phosphonates, including methylphosphonate, phosphorothioate, and phosphotriester modifications such as alkylphosphotriesters, and the like. Examples of modifications contemplated for the sugar moiety include 2′-alkyl pyrimidine, such as 2′-O-methyl, 2′-fluoro, amino, and deoxy modifications and the like (see, e.g., Amarzguioui et al., 2003). Examples of modifications contemplated for the base groups include abasic sugars, 2-O-alkyl modified pyrimidines, 4-thiouracil, 5-bromouracil, 5-iodouracil, and 5-(3-aminoallyl)-uracil and the like. Locked nucleic acids, or LNA's, could also be incorporated. Many other modifications are known and can be used so long as the above criteria are satisfied. Examples of modifications are also disclosed in U.S. Pat. Nos. 5,684,143, 5,858,988 and 6,291,438 and in U.S. published patent application No. 2004/0203145 A1. Other modifications are disclosed in Herdewijn (2000), Eckstein (2000), Rusckowski et al. (2000), Stein et al. (2001); Vorobjev et al. (2001).

One or more modifications contemplated can be incorporated into a nucleic acid strand of the molecules of the invention. The placement of the modifications in the DsiRNA molecule can greatly affect the characteristics of the DsiRNA molecule, including conferring greater potency and stability, reducing toxicity, enhance Dicer processing, and minimizing an immune response. In one embodiment, the antisense strand or the sense strand or both strands have one or more 2′-O-methyl modified nucleotides. In another embodiment, the antisense strand contains 2′-O-methyl modified nucleotides. In another embodiment, the antisense stand contains a 3′ overhang that is comprised of 2′-O-methyl modified nucleotides. The antisense strand could also include additional 2′-O-methyl modified nucleotides.

In certain embodiments of the present invention, the dsRNA region of the nucleic acid molecules of the invention has one or more properties which enhance its processing by Dicer. According to these embodiments, the Dicer substrate molecule has a length sufficient such that it is processed by Dicer to produce an active siRNA and at least one of the following properties: (i) the Dicer substrate molecule is asymmetric, e.g., has a 3′ overhang on the antisense strand and (ii) the Dicer substrate molecule has a modified 5′ end on the sense strand and a modified 3′ end on the antisense strand to direct orientation of Dicer binding and processing of the dsRNA region to an active siRNA. In certain such embodiments, the presence of the aptamer region itself can also serve to orient such a molecule for appropriate directionality of Dicer enzyme cleavage.

In some embodiments, the longest strand in the dsRNA region comprises 21-25, 25-30 nucleotides. In one embodiment, the Dicer substrate forms a structure such that the 3′ end of the antisense strand overhangs the 5′ end of the sense strand. In certain embodiments, the 3′ overhang of the antisense strand is 1-10 nucleotides, and optionally is 1-4 nucleotides, for example 2 nucleotides. Both the sense and the antisense strand may also have a 5′ phosphate.

In certain embodiments, the nucleic acid molecule of the invention may be modified by nucleotides such as deoxyribonucleotides, dideoxyribonucleotides, acyclonucleotides and the like and sterically hindered molecules, such as fluorescent molecules and the like. Acyclonucleotides substitute a 2-hydroxyethoxymethyl group for the 2′-deoxyribofuranosyl sugar normally present in dNMPs. Other nucleotide modifiers could include 3′-deoxyadenosine (cordycepin), 3′-azido-3′-deoxythymidine (AZT), 2′,3′-dideoxyinosine (ddI), 2′,3′-dideoxy-3′-thiacytidine (3TC), 2′,3′-didehydro-2′,3′-dideoxythymidine (d4T) and the monophosphate nucleotides of 3′-azido-3′-deoxythymidine (AZT), 2′,3′-dideoxy-3′-thiacytidine (3TC) and 2′,3′-didehydro-2′,3′-dideoxythymidine (d4T). In one embodiment, deoxynucleotides are used as the modifiers. When nucleotide modifiers are utilized, 1-3 nucleotide modifiers, or 2 nucleotide modifiers are substituted for the ribonucleotides on the 3′ end of the sense strand. When sterically hindered molecules are utilized, they are attached to the ribonucleotide at the 3′ end of the antisense strand. Thus, the length of the strand does not change with the incorporation of the modifiers. In another embodiment, the invention contemplates substituting two DNA bases in the Dicer substrate molecule to direct the orientation of Dicer processing of the antisense strand. In a further embodiment of the present invention, two terminal DNA bases are substituted for two ribonucleotides on the 3′-end of the sense strand forming a blunt end of the duplex on the 3′ end of the sense strand and the 5′ end of the antisense strand, and a two-nucleotide RNA overhang is located on the 3′-end of the antisense strand. This is an asymmetric composition with DNA on the blunt end and RNA bases on the overhanging end. In certain embodiments of the instant invention, the modified nucleotides (e.g., deoxyribonucleotides) of the penultimate and ultimate positions of the 3′ terminus of the antisense strand base pair with corresponding modified nucleotides (e.g., deoxyribonucleotides) of the sense strand (optionally, the penultimate and ultimate residues of the 5′ end of the antisense strand in those Dicer substrate molecules of the instant invention possessing a blunt end at the 3′ terminus of the sense strand/5′ terminus of the antisense strand).

The strand(s) of a nucleic acid molecule of the instant invention can anneal or adopt secondary/tertiary structure under biological conditions, such as the conditions found in the cytoplasm of a cell. The sense and antisense strands of the Dicer substrate in the nucleic acid molecule of the instant invention anneal under biological conditions, such as the conditions found in the cytoplasm of a cell. In addition, a region of one of the sequences, particularly of the antisense strand, of the Dicer substrate molecule has a sequence length of at least 19 nucleotides, wherein these nucleotides are in the 21-nucleotide region adjacent to the 3′ end of the antisense strand and are sufficiently complementary to a nucleotide sequence of the RNA produced from the target gene to anneal with and/or decrease levels of such a target RNA.

The Dicer substrate portion of the nucleic acid molecule of the invention may also have one or more of the following additional properties: (a) the antisense strand has a right or left shift from the typical 21 mer, (b) the strands may not be completely complementary, i.e., the strands may contain simple mismatch pairings and (c) base modifications such as locked nucleic acid(s) may be included in the 5′ end of the sense strand. A “typical” 21 mer siRNA is designed using conventional techniques. In one technique, a variety of sites are commonly tested in parallel or pools containing several distinct siRNA duplexes specific to the same target with the hope that one of the remolecules will be effective (Ji et al., 2003). Other techniques use design rules and algorithms to increase the likelihood of obtaining active RNAi effector molecules (Schwarz et al., 2003; Khvorova et al., 2003; Ui-Tei et al., 2004; Reynolds et al., 2004; Krol et al., 2004; Yuan et al., 2004; Boese et al., 2005). High throughput selection of siRNA has also been developed (U.S. published patent application No. 2005/0042641 A1). Potential target sites can also be analyzed by secondary structure predictions (Heale et al., 2005). This 21 mer is then used to design a right shift to include 3-9 additional nucleotides on the 5′ end of the 21 mer. The sequence of these additional nucleotides may have any sequence. In one embodiment, the added ribonucleotides are based on the sequence of the target gene. Even in this embodiment, full complementarity between the target sequence and the antisense siRNA is not required.

The first and second oligonucleotides of a Dicer substrate portion of the nucleic acid molecule of the instant invention are not required to be completely complementary. They only need to be substantially complementary to anneal under biological conditions and to provide a substrate for Dicer that produces a siRNA sufficiently complementary to the target sequence. Locked nucleic acids, or LNA's, are well known to a skilled artisan (Elman et al., 2005; Kurreck et al., 2002; Crinelli et al., 2002; Braasch and Corey, 2001; Bondensgaard et al., 2000; Wahlestedt et al., 2000). In one embodiment, an LNA is incorporated at the 5′ terminus of the sense strand. In another embodiment, an LNA is incorporated at the 5′ terminus of the sense strand in duplexes designed to include a 3′ overhang on the antisense strand.

Certain Dicer substrate molecule compositions of the invention contain two separate oligonucleotides can be linked by a third structure (e.g., an aptamer). The third structure will not block Dicer activity on the Dicer substrate molecule and will not interfere with the directed destruction of the RNA transcribed from the target gene. In one embodiment, the third structure is a nucleic acid aptamer. The nucleic acid aptamer links the two oligonucleotides of the Dicer substrate molecule in a manner such that a, e.g., hairpin, structure is produced upon annealing of the two oligonucleotides making up the dsRNA composition. Many suitable chemical linking groups are known in the art and can be used. Preferably, the Dicer substrate molecule of the invention is connected to the aptamer by a backbone (e.g., a phosphodiester backbone). Alternatively, the third structure may be a polypeptide aptamer. The polypeptide aptamer will not block Dicer activity on the Dicer substrate molecule and may itself be processed by Dicer.

In Dicer substrate compositions of the invention, the sense and antisense strands anneal under biological conditions, such as the conditions found in the cytoplasm of a cell. In addition, a region of one of the sequences, particularly of the antisense strand, of the dsRNA region has a sequence length of at least 19 nucleotides, wherein these nucleotides are adjacent to the 3′ end of antisense strand and are sufficiently complementary to a nucleotide sequence of the target RNA to direct RNA interference.

Additionally, the Dicer substrate aptamer of the invention can be optimized to ensure that the oligonucleotide segment generated from Dicer's cleavage will be the portion of the oligonucleotide that is most effective in inhibiting gene expression. For example, in one embodiment of the invention, a 27-35-bp oligonucleotide of the Dicer substrate is incorporated into the design of the stem wherein the anticipated 21 to 22-bp segment that will inhibit gene expression is located on the 3′-end of the antisense strand. The remaining bases located on the 5′-end of the antisense strand will be cleaved by Dicer and will be discarded. This cleaved portion can be homologous (i.e., based on the sequence of the target sequence) or non-homologous and added to extend the nucleic acid strand.

US 2007/0265220 discloses that 27mer DsiRNAs show improved stability in serum over comparable 21 mer siRNA compositions, even absent chemical modification. Modifications of the Dicer substrate portion of the aptamer molecules of the invention, such as inclusion of 2′-O-methyl RNA in the antisense strand, in patterns such as detailed in US 2007/0265220 and in the instant Examples, when coupled with addition of a 5′ Phosphate, can improve stability of DsiRNA molecules. Addition of 5′-phosphate to all strands in synthetic RNA duplexes may be an inexpensive and physiological method to confer some limited degree of nuclease stability.

The chemical modification patterns in the receptor binding region of the nucleic acid molecules of the instant invention are designed to enhance the efficacy of such molecules. Accordingly, such modifications are designed to avoid reducing potency of Dicer substrates; to avoid interfering with Dicer processing of DsiRNAs; to improve stability in biological fluids (reduce nuclease sensitivity) of DsiRNAs; or to block or evade detection by the innate immune system. Such modifications are also designed to avoid being toxic and to avoid increasing the cost or impact the ease of manufacturing the instant DsiRNA molecules of the invention.

In certain specific embodiments, the receptor binding Dicer substrate molecules of the invention can have the following structures:

In one such embodiment, the receptor binding Dicer substrate molecule comprises:

5′ -XXXXXXXXXXXXXXXXXXXXXXXXX (Rb) -3′
3′ -YXXXXXXXXXXXXXXXXXXXXXXXXX (Rb) -5′

wherein “X”=RNA, “p”=a phosphate group and “Y” is an overhang domain comprised of 1-4 RNA monomers that are optionally 2′-O-methyl RNA monomers. In a related embodiment, the Dicer substrate comprises:

5′ -XXXXXXXXXXXXXXXXXXXXXXXDD (Rb) -3′
3′ -YXXXXXXXXXXXXXXXXXXXXXXXXX (Rb) -5′

wherein “X”=RNA, “Y” is an overhang domain comprised of 1-4 RNA monomers that are optionally 2′-O-methyl RNA monomers, “D”=DNA, and “(Rb)” denotes additional nucleotides capable of conferring receptor binding properties to the receptor binding Dicer substrate as a composition. Optionally, the residues denoted as “(Rb)” residues form a continuous stretch of residues such that the single 5′ terminus of the receptor binding Dicer substrate molecule is the 5′ terminus of the top strand above and the single 3′ terminus of the receptor binding Dicer substrate molecule is the 3′ terminus of the bottom strand above. In one embodiment, the top strand is the sense strand, and the bottom strand is the antisense strand. Alternatively, the bottom strand is the sense strand and the top strand is the antisense strand.

In additional such embodiments, the receptor binding Dicer substrate molecule comprises:

5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -YXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -YXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -YXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -YXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -YXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -YXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -YXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -YXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -YXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -YXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -YXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -YXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -YXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -YXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -YXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -YXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -YXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -YXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -YXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -YXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -YXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -YXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -YXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -YXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -YXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -YXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -YXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -YXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -YXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -YXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -YXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -YXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -YXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -YXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -YXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -YXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -YXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -YXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -YXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -YXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -YXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -YXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -YXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -YXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -YXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -YXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXXX (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -YXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′
or
5′ -XXXXXXXXXXXXXXXXXXXXXXXDD (Rb) -3′
3′ -XXXXXXXXXXXXXXXXXXXXXXXXXXX (Rb) -5′

wherein “X”=RNA, “Y” is an overhang domain comprised of 1-4 RNA monomers that are optionally 2′-O-methyl RNA monomers, “D”=DNA, and “(Rb)” denotes additional nucleotides capable of conferring receptor binding properties to the receptor binding Dicer substrate as a composition. Optionally, the residues denoted as “(Rb)” residues form a continuous stretch of residues such that the single 5′ terminus of the receptor binding Dicer substrate molecule is the 5′ terminus of the top strand above and the single 3′ terminus of the receptor binding Dicer substrate molecule is the 3′ terminus of the bottom strand above. In one embodiment, the top strand is the sense strand, and the bottom strand is the antisense strand. Alternatively, the bottom strand is the sense strand and the top strand is the antisense strand.

### Use of Nucleic Acid Molecules According to the Invention

### Targeting and Delivery of Dicer Substrate Molecules

In certain embodiments, the present invention relates to a method for treating a subject having or at risk of developing a disease or disorder. In such embodiments, the nucleic acid molecule of the invention can act as a novel therapeutic molecule for controlling the disease or disorder. The method comprises administering a pharmaceutical composition of the invention to the patient (e.g., human), such that the expression, level and/or activity a target RNA is reduced. The expression, level and/or activity of a polypeptide encoded by the target RNA might also be reduced by a nucleic acid molecule of the instant invention containing a Dicer substrate and a nucleic acid aptamer that binds a receptor.

In the treatment of a disease or disorder, the nucleic acid molecule of the invention can be brought into contact with the cells or tissue exhibiting or associated with a disease or disorder. For example, nucleic acid molecule of the invention containing a Dicer substrate substantially identical to all or part of a target RNA sequence, may be brought into contact with or introduced into a diseased, disease-associated or infected cell, either in vivo or in vitro. Similarly, nucleic acid molecules of the invention containing a Dicer substrate substantially identical to all or part of a target RNA sequence may administered directly to a subject having or at risk of developing a disease or disorder.

Therapeutic use of the nucleic acid molecules of the instant invention can involve use of formulations of nucleic acid molecules comprising multiple different antisense sequences. For example, two or more, three or more, four or more, five or more, etc. of the presently described molecules can be combined to produce a formulation that, e.g., targets multiple different regions of one or more target RNA(s). A nucleic acid molecule of the instant invention containing a Dicer substrate may also be constructed such that either strand of the Dicer substrate molecule independently targets two or more regions of a target RNA. Use of such a multifunctional Dicer substrate that targets more then one region of a target nucleic acid molecule is expected to provide potent inhibition of RNA levels and expression. For example, a nucleic acid molecule containing a single multifunctional Dicer substrate construct of the invention can target both conserved and variable regions of a target nucleic acid molecule, thereby allowing down regulation or inhibition of, e.g., different strain variants of a virus, or splice variants encoded by a single target gene.

A nucleic acid molecule of the invention can be unconjugated or conjugated (e.g., at its 5′ or 3′ terminus of its sense or antisense strand) to another moiety (e.g. a non-nucleic acid moiety such as a peptide), an organic compound (e.g., a dye, cholesterol, or the like). Modifying nucleic acid molecules in this way may improve cellular uptake or enhance cellular targeting activities of the resulting nucleic acid molecule derivative as compared to the corresponding unconjugated nucleic acid molecule, are useful for tracing the nucleic acid molecule derivative in the cell, or improve the stability of the nucleic acid molecule derivative compared to the corresponding unconjugated nucleic acid molecule. Without being bound to any one mechanism, the nucleic acid molecule of the invention crosses the plasma membrane and is internalized. An assay to measure internalization involves contacting a fluorescently labeled nucleic acid molecule of the invention with a receptor on the surface of a cell and observing the presence of the fluorescent label in the cell relative to an appropriate reference. Preferably, the label is attached to the molecule that does not interfere with receptor binding. To measure internalization of a nucleic acid molecule of the invention, a cell that does not have Dicer processing activity may be used. Such internalization assays can be performed in a SELEX method involving receptors on whole cells (Hicke et al. Biol Chem. 2001 Dec. 28; 276(52):48644-54, Daniels et al., Anal Biochem. 2002 Jun. 15; 305(2):214-26, and Daniels et al. Proc Natl Acad Sci USA. 2003 Dec. 23; 100(26):15416-212003, which are herein incorporated by reference).

### RNAi In Vitro Assay to Assess Dicer Substrate Activity

An in vitro assay that recapitulates RNAi in a cell-free system can optionally be used to evaluate nucleic acid molecules of the invention containing a Dicer substrate molecule and a receptor-binding region. For example, such an assay comprises a system described by Tuschl et al., 1999, Genes and Development, 13, 3191-3197 and Zamore et al., 2000, Cell, 101, 25-33, adapted for use with nucleic acid molecules of the invention containing Dicer substrate molecules directed against target RNA. A Drosophila extract derived from syncytial blastoderm is used to reconstitute RNAi activity in vitro. Target RNA is generated via in vitro transcription from an appropriate plasmid using T7 RNA polymerase or via chemical synthesis. If the aptamer is formed by a single polynucleotide strand, the polynucleotide strand (for example 20 uM each) is incubated in buffer (such as 100 mM potassium acetate, 30 mM HEPES-KOH, pH 7.4, 2 mM magnesium acetate) for 1 minute at 90° C. followed by 1 hour at 37° C., then diluted in lysis buffer (for example 100 mM potassium acetate, 30 mM HEPES-KOH at pH 7.4, 2 mM magnesium acetate). If the nucleic acid molecule is formed by two polynucleotide strands, the two strands (for example 20 uM each) are annealed by incubation in buffer (such as 100 mM potassium acetate, 30 mM HEPES-KOH, pH 7.4, 2 mM magnesium acetate) for 1 minute at 90° C. followed by 1 hour at 37° C., then diluted in lysis buffer (for example 100 mM potassium acetate, 30 mM HEPES-KOH at pH 7.4, 2 mM magnesium acetate). Annealing can be monitored by gel electrophoresis on an agarose gel in TBE buffer and stained with ethidium bromide. The Drosophila lysate is prepared using zero to two-hour-old embryos from Oregon R flies collected on yeasted molasses agar that are dechorionated and lysed. The lysate is centrifuged and the supernatant isolated. The assay comprises a reaction mixture containing 50% lysate [vol/vol], RNA (10-50 pM final concentration), and 10% [vol/vol] lysis buffer containing Dicer substrate (10 nM final concentration). The reaction mixture also contains 10 mM creatine phosphate, 10 ug/ml creatine phosphokinase, 100 μM GTP, 100 μM UTP, 100 μM CTP, 500 μM ATP, 5 mM DTT, 0.1 U/uL RNasin (Promega), and 100 uM of each amino acid. The final concentration of potassium acetate is adjusted to 100 mM. The reactions are pre-assembled on ice and preincubated at 25° C. for 10 minutes before adding the nucleic acid components, then incubated at 25° C. for an additional 60 minutes. Reactions are quenched with 4 volumes of 1.25× Passive Lysis Buffer (Promega). Target RNA cleavage is assayed by RT-PCR analysis or other methods known in the art and are compared to control reactions, e.g., in which the receptor binding region is omitted from the reaction.

Alternately, internally-labeled target RNA for the assay is prepared by in vitro transcription in the presence of [alpha-32P] CTP, passed over a G50 Sephadex column by spin chromatography and used as target RNA without further purification. Optionally, target RNA is 5′-32P-end labeled using T4 polynucleotide kinase enzyme. Assays are performed as described above and target RNA and the specific RNA cleavage products generated by RNAi are visualized on an autoradiograph of a gel. The percentage of cleavage is determined by PHOSPHOR IMAGER® (autoradiography) quantitation of bands representing intact control RNA or RNA from control reactions without the receptor binding region and the cleavage products generated by the assay.

### Methods of Introducing Nucleic Acids, Vectors, and Host Cells

Nucleic acid molecules of the invention containing a Dicer substrate and a receptor-binding region may be directly introduced into a cell (i.e., intracellularly); or introduced extracellularly into a cavity, interstitial space, into the circulation of an organism, introduced orally, or may be introduced by bathing a cell or organism in a solution containing the nucleic acid. Vascular or extravascular circulation, the blood or lymph system, and the cerebrospinal fluid are sites where the nucleic acid may be introduced.

Nucleic acid molecules of the invention containing a Dicer substrate and a receptor-binding region can be introduced using nucleic acid delivery methods known in art including injection of a solution containing the nucleic acid. An advantage of the invention is that the receptor binding region of the nucleic acid molecule may be designed to bind a cell surface receptor which is internalized into the cell, thereby simplifying delivery formulations. Alternatively, delivery of the nucleic acid molecules of the invention include bombardment by particles covered by the nucleic acid, soaking the cell or organism in a solution of the nucleic acid, or electroporation of cell membranes in the presence of the nucleic acid. Other methods known in the art for introducing nucleic acids to cells may be used, such as lipid-mediated carrier transport, chemical-mediated transport, and cationic liposome transfection such as calcium phosphate, and the like. The nucleic acid may be introduced along with other components that perform one or more of the following activities: enhance nucleic acid uptake by the cell or otherwise increase inhibition of the target RNA.

A cell having a target RNA may be from the germ line or somatic, totipotent or pluripotent, dividing or non-dividing, parenchyma or epithelium, immortalized or transformed, or the like. The cell may be a stem cell or a differentiated cell. Cell types that are differentiated include adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes, and cells of the endocrine or exocrine glands.

Depending on the particular target RNA sequence and the dose of the nucleic acid molecule of the invention delivered, this process may provide partial or complete loss of function for the target RNA. A reduction or loss of RNA levels or expression (either RNA expression or encoded polypeptide expression) in at least 50%, 60%, 70%, 80%, 90%, 95% or 99% or more of targeted cells is exemplary Inhibition of target RNA levels or expression refers to the absence (or observable decrease) in the level of RNA or RNA-encoded protein. Specificity refers to the ability to inhibit the target RNA without manifest effects on other genes of the cell. The consequences of inhibition can be confirmed by examination of the outward properties of the cell or organism (as presented below in the examples) or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). Inhibition of target RNA sequence(s) by the nucleic acid molecules of the invention also can be measured based upon the effect of administration of such aptamer molecules upon measurable phenotypes such as tumor size for cancer treatment, viral load/titer for viral infectious diseases, etc. either in vivo or in vitro. For viral infectious diseases, reductions in viral load or titer can include reductions of, e.g., 50%, 60%, 70%, 80%, 90%, 95% or 99% or more, and are often measured in logarithmic terms, e.g., 10-fold, 100-fold, 1000-fold, 10⁵-fold, 10⁶-fold, 10⁷-fold reduction in viral load or titer can be achieved via administration of the nucleic acid moleculess of the invention to cells, a tissue, or a subject.

For RNA-mediated inhibition in a cell line or whole organism, expression of a reporter or drug resistance gene whose protein product is easily assayed can be measured. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracyclin. Depending on the assay, quantitation of the amount of gene expression allows one to determine a degree of inhibition which is greater than 10%, 33%, 50%, 90%, 95% or 99% as compared to a cell not treated according to the present invention.

Lower doses of injected material and longer times after administration of an RNA silencing molecule of the invention may result in inhibition in a smaller fraction of cells (e.g., at least 10%, 20%, 50%, 75%, 90%, or 95% of targeted cells). Quantitation of gene expression in a cell may show similar amounts of inhibition at the level of accumulation of target RNA or translation of target protein. As an example, the efficiency of inhibition may be determined by assessing the amount of gene product in the cell; RNA may be detected with a hybridization probe having a nucleotide sequence outside the region used for the inhibitory Dicer substrate, or translated polypeptide may be detected with an antibody raised against the polypeptide sequence of that region.

Nucleic acid molecules of the invention containing a Dicer substrate and a receptor-binding region may be introduced in an amount which allows delivery of at least one copy per cell. Higher doses (e.g., at least 5, 10, 100, 500 or 1000 copies per cell) of material may yield more effective inhibition; lower doses may also be useful for specific applications.

### RNA Interference Based Therapy

As is known, RNAi methods are applicable to a wide variety of genes in a wide variety of organisms and the disclosed compositions and methods can be utilized in each of these contexts. Examples of genes which can be targeted by the disclosed compositions and methods include endogenous genes which are genes that are native to the cell or to genes that are not normally native to the cell. Without limitation, these genes include oncogenes, cytokine genes, idiotype (Id) protein genes, prion genes, genes that expresses molecules that induce angiogenesis, genes for adhesion molecules, cell surface receptors, proteins involved in metastasis, proteases, apoptosis genes, cell cycle control genes, genes that express EGF and the EGF receptor, multi-drug resistance genes, such as the MDR1 gene.

More specifically, a target mRNA of the invention can specify the amino acid sequence of a cellular protein (e.g., a nuclear, cytoplasmic, transmembrane, or membrane-associated protein). In another embodiment, the target mRNA of the invention can specify the amino acid sequence of an extracellular protein (e.g., an extracellular matrix protein or secreted protein). As used herein, the phrase “specifies the amino acid sequence” of a protein means that the mRNA sequence is translated into the amino acid sequence according to the rules of the genetic code. The following classes of proteins are listed for illustrative purposes: developmental proteins (e.g., adhesion molecules, cyclin kinase inhibitors, Wnt family members, Pax family members, Winged helix family members, Hox family members, cytokines/lymphokines and their receptors, growth/differentiation factors and their receptors, neurotransmitters and their receptors); oncogene-encoded proteins (e.g., ABLI, BCLI, BCL2, BCL6, CBFA2, CBL, CSFIR, ERBA, ERBB, EBRB2, ETSI, ETSI, ETV6, FGR, FOS, FYN, HCR, HRAS, JUN, KRAS, LCK, LYN, MDM2, MLL, MYB, MYC, MYCLI, MYCN, NRAS, PIM I, PML, RET, SRC, TALI, TCL3, and YES); tumor suppressor proteins (e.g., APC, BRCA1, BRCA2, MADH4, MCC, NFI, NF2, RB I, TP53, and WTI); and enzymes (e.g., ACC synthases and oxidases, ACP desaturases and hydroxylases, ADP-glucose pyrophorylases, ATPases, alcohol dehydrogenases, amylases, amyloglucosidases, catalases, cellulases, chalcone synthases, chitinases, cyclooxygenases, decarboxylases, dextriinases, DNA and RNA polymerases, galactosidases, glucanases, glucose oxidases, granule-bound starch synthases, GTPases, helicases, hernicellulases, integrases, inulinases, invertases, isomerases, kinases, lactases, lipases, lipoxygenases, lysozymes, nopaline synthases, octopine synthases, pectinesterases, peroxidases, phosphatases, phospholipases, phosphorylases, phytases, plant growth regulator synthases, polygalacturonases, proteinases and peptidases, pullanases, recombinases, reverse transcriptases, RUBISCOs, topoisomerases, and xylanases).

In one aspect, the target mRNA molecule of the invention specifies the amino acid sequence of a protein associated with a pathological condition. For example, the protein may be a pathogen-associated protein (e.g., a viral protein involved in immunosuppression of the host, replication of the pathogen, transmission of the pathogen, or maintenance of the infection), or a host protein which facilitates entry of the pathogen into the host, drug metabolism by the pathogen or host, replication or integration of the pathogen's genome, establishment or spread of infection in the host, or assembly of the next generation of pathogen. Pathogens include RNA viruses such as flaviviruses, picornaviruses, rhabdoviruses, filoviruses, retroviruses, including lentiviruses, or DNA viruses such as adenoviruses, poxviruses, herpes viruses, cytomegaloviruses, hepadnaviruses or others. Additional pathogens include bacteria, fungi, helminths, schistosomes and trypanosomes. Other kinds of pathogens can include mammalian transposable elements. Alternatively, the protein may be a tumor-associated protein or an autoimmune disease-associated protein.

The target gene may be derived from or contained in any organism. The organism may be a plant, animal, protozoa, bacterium, virus or fungus. See e.g., U.S. Pat. No. 6,506,559, incorporated herein by reference.

### Pharmaceutical Compositions

In certain embodiments, the present invention provides for a pharmaceutical composition comprising the nucleic acid molecule of the present invention. The nucleic acid molecule sample can be suitably formulated and introduced into the environment of the cell by any means that allows for a sufficient portion of the sample to enter the cell to induce gene silencing, if it is to occur. Many formulations for introducing polynucleotides are known in the art and can be used so long as the nucleic acid molecule of the invention gains entry to the target cells so that it can act. See, e.g., U.S. published patent application Nos. 2004/0203145 A1 and 2005/0054598 A1. For example, the nucleic acid molecule of the instant invention can be formulated in buffer solutions such as phosphate buffered saline solutions, liposomes, micellar structures, and capsids. Because of the receptor binding properties of the nucleic acid molecule of the invention, the nucleic acid molecule of the invention can be formulated into a pharmaceutically acceptable carrier (e.g., a suitable buffer solution) without the need for further delivery molecules (e.g., cationic lipids). Nevertheless, formulations of the nucleic acid molecule of the invention with cationic lipids can be used to facilitate transfection of the nucleic acid molecule into cells. For example, cationic lipids, such as lipofectin (U.S. Pat. No. 5,705,188), cationic glycerol derivatives, and polycationic molecules, such as polylysine (published PCT International Application WO 97/30731), can be used. Suitable lipids include Oligofectamine, Lipofectamine (Life Technologies), NC388 (Ribozyme Pharmaceuticals, Inc., Boulder, Colo.), or FuGene 6 (Roche) all of which can be used according to the manufacturer's instructions.

Such compositions typically include the nucleic acid molecule of the invention and a pharmaceutically acceptable carrier. As used herein the language “pharmaceutically acceptable carrier” includes saline, solvents, dispersion media, coatings, antibacterial and antifungal molecules, isotonic and absorption delaying molecules, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial molecules such as benzyl alcohol or methyl parabens; antioxidants such as ascorbic acid or sodium bisulfite; chelating molecules such as ethylenediaminetetraacetic acid; buffers such as acetates, citrates or phosphates and molecules for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal molecules, for example, parabens, chlorobutanol, phenol, ascorbic acid, thimerosal, and the like. In many cases, it will be preferable to include isotonic molecules, for example, sugars, polyalcohols such as manitol, sorbitol, sodium chloride in the composition. Prolonged absorption of the injectable compositions can be brought about by including in the composition an molecule which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof. Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding molecules, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating molecule such as alginic acid, Primogel, or corn starch; a lubricant such as magnesium stearate or Sterotes; a glidant such as colloidal silicon dioxide; a sweetening molecule such as sucrose or saccharin; or a flavoring molecule such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer. Such methods include those described in U.S. Pat. No. 6,468,798.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

The compounds can also be administered by transfection or infection using methods known in the art, including but not limited to the methods described in McCaffrey et al. (2002), Nature, 418(6893), 38-9 (hydrodynamic transfection); Xia et al. (2002), Nature Biotechnol., 20(10), 1006-10 (viral-mediated delivery); or Putnam (1996), Am. J. Health Syst. Pharm. 53(2), 151-160, erratum at Am. J. Health Syst. Pharm. 53(3), 325 (1996).

The compounds can also be administered by any method suitable for administration of nucleic acid molecules, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al. (1998), Clin. Immunol. Immunopathol., 88(2), 205-10. Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of a nucleic acid molecule (i.e., an effective dosage) depends on the nucleic acid selected. For instance, if a plasmid encoding a nucleic acid molecule of the invention is selected, single dose amounts in the range of approximately 1 pg to 1000 mg may be administered; in some embodiments, 10, 30, 100, or 1000 pg, or 10, 30, 100, or 1000 ng, or 10, 30, 100, or 1000 μg, or 10, 30, 100, or 1000 mg may be administered. In some embodiments, 1-5 g of the compositions can be administered. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

It can be appreciated that the method of introducing nucleic acid molecule of the invention into the environment of the cell will depend on the type of cell and the make up of its environment. For example, when the cells are found within a liquid, one preferable formulation is with an aqueous formulation containing the nucleic acid molecule of the invention which is added directly to the liquid environment of the cells. Aqueous formulations can also be administered to animals such as by intravenous, intramuscular, or intraperitoneal injection, or orally or by inhalation or other methods as are known in the art. Another preferable formulation is with a lipid formulation such as in lipofectamine and the Dicer substrate molecules can be added directly to the liquid environment of the cells. Lipid formulations can also be administered to animals such as by intravenous, intramuscular, or intraperitoneal injection, or orally or by inhalation or other methods as are known in the art. When the formulation is suitable for administration into animals such as mammals and more specifically humans, the formulation is also pharmaceutically acceptable. Pharmaceutically acceptable formulations for administering oligonucleotides are known and can be used. In some instances, it may be preferable to formulate nucleic acid molecules of the invention in a buffer or saline solution and directly inject the formulated nucleic acid molecules of the invention into cells. The direct injection of nucleic acid molecules of the invention may also be done. For suitable methods of introducing nucleic acid molecules (e.g., nucleic acid molecule of the invention), see U.S. published patent application No. 2004/0203145 A1.

Suitable amounts of a nucleic acid molecule of the invention must be introduced and these amounts can be empirically determined using standard methods. Typically, effective concentrations of individual nucleic acid molecule species in the environment of a cell will be about 50 nanomolar or less, 10 nanomolar or less, or compositions in which concentrations of about 1 nanomolar or less can be used. In another embodiment, methods utilizing a concentration of about 200 picomolar or less, and even a concentration of about 50 picomolar or less, about 20 picomolar or less, about 10 picomolar or less, or about 5 picomolar or less can be used in many circumstances.

The method can be carried out by addition of the nucleic acid molecule compositions to any extracellular matrix in which cells can live provided that the nucleic acid molecule composition is formulated so that a sufficient amount of the Dicer substrate molecule can enter the cell to exert its effect. For example, the method is amenable for use with cells present in a liquid such as a liquid culture or cell growth media, in tissue explants, or in whole organisms, including animals, such as mammals and especially humans.

The level or activity of a target RNA can be determined by any suitable method now known in the art or that is later developed. It can be appreciated that the method used to measure a target RNA and/or the expression of a target RNA can depend upon the nature of the target RNA. For example, if the target RNA encodes a protein, the term “expression” can refer to a protein or the RNA/transcript derived from the target RNA. In such instances, the expression of a target RNA can be determined by measuring the amount of RNA corresponding to the target RNA or by measuring the amount of that protein. Protein can be measured in protein assays such as by staining or immunoblotting or, if the protein catalyzes a reaction that can be measured, by measuring reaction rates. All such methods are known in the art and can be used. Where target RNA levels are to be measured, any art-recognized methods for detecting RNA levels can be used (e.g., RT-PCR, Northern Blotting, etc.). In targeting viral RNAs with the nucleic acid molecule of the instant invention, it is also anticipated that measurement of the efficacy of a nucleic acid molecule of the invention in reducing levels of a target virus in a subject, tissue, in cells, either in vitro or in vivo, or in cell extracts can also be used to determine the extent of reduction of target viral RNA level(s). Any of the above measurements can be made on cells, cell extracts, tissues, tissue extracts or any other suitable source material.

The determination of whether the expression of a target RNA has been reduced can be by any suitable method that can reliably detect changes in RNA levels. Typically, the determination is made by introducing into the environment of a cell an undigested nucleic acid molecule of the invention such that at least a portion of that nucleic acid molecule of the invention enters the cytoplasm, and then measuring the level of the target RNA. The same measurement is made on identical untreated cells and the results obtained from each measurement are compared.

The nucleic acid molecule of the invention can be formulated as a pharmaceutical composition which comprises a pharmacologically effective amount of a nucleic acid molecule of the invention and pharmaceutically acceptable carrier. A pharmacologically or therapeutically effective amount refers to that amount of a nucleic acid molecule of the invention effective to produce the intended pharmacological, therapeutic or preventive result. The phrases “pharmacologically effective amount” and “therapeutically effective amount” or simply “effective amount” refer to that amount of an nucleic acid molecule effective to produce the intended pharmacological, therapeutic or preventive result. For example, if a given clinical treatment is considered effective when there is at least a 20% reduction in a measurable parameter associated with a disease or disorder, a therapeutically effective amount of a drug for the treatment of that disease or disorder is the amount necessary to effect at least a 20% reduction in that parameter.

Suitably formulated pharmaceutical compositions of this invention can be administered by any means known in the art such as by parenteral routes, including intravenous, intramuscular, intraperitoneal, subcutaneous, transdermal, airway (aerosol), rectal, vaginal and topical (including buccal and sublingual) administration. In some embodiments, the pharmaceutical compositions are administered by intravenous or intraparenteral infusion or injection.

In general, a suitable dosage unit of a nucleic acid molecule of the invention will be in the range of 0.001 to 0.25 milligrams per kilogram body weight of the recipient per day, or in the range of 0.01 to 20 micrograms per kilogram body weight per day, or in the range of 0.01 to 10 micrograms per kilogram body weight per day, or in the range of 0.10 to 5 micrograms per kilogram body weight per day, or in the range of 0.1 to 2.5 micrograms per kilogram body weight per day. Pharmaceutical composition comprising the nucleic acid molecule of the invention can be administered once daily. However, the therapeutic molecule may also be dosed in dosage units containing two, three, four, five, six or more sub-doses administered at appropriate intervals throughout the day. In that case, the nucleic acid molecule of the invention contained in each sub-dose must be correspondingly smaller in order to achieve the total daily dosage unit. The dosage unit can also be compounded for a single dose over several days, e.g., using a conventional sustained release formulation which provides sustained and consistent release of the nucleic acid molecule of the invention over a several day period. Sustained release formulations are well known in the art. In this embodiment, the dosage unit contains a corresponding multiple of the daily dose. Regardless of the formulation, the pharmaceutical composition must contain the nucleic acid molecule of the invention in a quantity sufficient to inhibit expression of the target gene in the animal or human being treated. The composition can be compounded in such a way that the sum of the multiple units of the nucleic acid molecule of the invention together contain a sufficient dose.

Data can be obtained from cell culture assays and animal studies to formulate a suitable dosage range for humans. The dosage of compositions of the invention lies within a range of circulating concentrations that include the ED₅₀ (as determined by known methods) with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range of the compound that includes the IC₅₀ (i.e., the concentration of the test compound which achieves a half-maximal inhibition of symptoms) as determined in cell culture. Such information can be used to more accurately determine useful doses in humans. Levels of a nucleic acid molecule of the invention in plasma may be measured by standard methods, for example, by high performance liquid chromatography.

The pharmaceutical compositions can be included in a kit, container, pack, or dispenser together with instructions for administration.

### Methods of Treatment

The present invention provides for both prophylactic and therapeutic methods of treating a subject at risk of (or susceptible to) a disease or disorder caused, in whole or in part, by the expression of a target RNA and/or the presence of such target RNA (e.g., in the context of a viral infection, the presence of a target RNA of the viral genome, capsid, host cell component, etc.).

“Treatment”, or “treating” as used herein, is defined as the application or administration of a therapeutic molecule (e.g., a nucleic acid molecule of the invention or vector or transgene encoding same) to a patient, or application or administration of a therapeutic molecule to an isolated tissue or cell line from a patient, who has the disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

In one aspect, the invention provides a method for preventing in a subject, a disease or disorder as described above, by administering to the subject a therapeutic molecule (e.g., a nucleic acid molecule of the invention or vector or transgene encoding same). Subjects at risk for the disease can be identified by, for example, any or a combination of diagnostic or prognostic assays as described herein. Administration of a prophylactic molecule can occur prior to the detection of, e.g., viral particles in a subject, or the manifestation of symptoms characteristic of the disease or disorder, such that the disease or disorder is prevented or, alternatively, delayed in its progression.

Another aspect of the invention pertains to methods of treating subjects therapeutically, i.e., alter onset of symptoms of the disease or disorder. These methods can be performed in vitro (e.g., by culturing the cell with the nucleic molecule of the invention) or, alternatively, in vivo (e.g., by administering the nucleic acid molecule of the invention to a subject).

With regard to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment with either the target RNA molecules of the present invention or target RNA modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

Therapeutic molecules can be tested in an appropriate animal model. For example, a nucleic acid molecule (or expression vector or transgene encoding same) as described herein can be used in an animal model to determine the efficacy, toxicity, or side effects of treatment with said molecule. Alternatively, a therapeutic molecule can be used in an animal model to determine the mechanism of action of such an molecule. For example, an molecule can be used in an animal model to determine the efficacy, toxicity, or side effects of treatment with such an molecule. Alternatively, an molecule can be used in an animal model to determine the mechanism of action of such an molecule.

Models Useful to Evaluate the Down-Regulation of mRNA Levels and Expression

### Cell Culture

The nucleic acid molecules of the invention can be tested for cleavage activity in vivo, for example, using the following procedure.

The Dicer substrate aptamers of the invention can be tested in cell culture using HeLa or other mammalian cells to determine the extent of target RNA and target protein inhibition. Dicer substrate aptamers (e.g., see FIGS. 1-4) are selected against the target as described herein. Target RNA inhibition is measured after delivery of these remolecules by a suitable transfection molecule to, for example, cultured HeLa cells or other transformed or non-transformed mammalian cells in culture. Relative amounts of target RNA are measured versus actin or other appropriate control using real-time PCR monitoring of amplification (e.g., ABI 7700 TAQMAN®). A comparison is made to a mixture of oligonucleotide sequences made to unrelated targets or to a randomized Dicer substrate control with the same overall length and chemistry, but randomly substituted at each position, or simply to appropriate vehicle-treated or untreated controls. Primary and secondary lead remolecules are chosen for the target and optimization performed. After an optimal transfection molecule concentration is chosen, a RNA time-course of inhibition is performed with the lead Dicer substrate molecule.

TAQMAN® (Real-Time PCR Monitoring of Amplification) and Lightcycler Quantification of mRNA

Total RNA is prepared from cells following Dicer substrate aptamer delivery, for example, using Ambion Rnaqueous 4-PCR purification kit for large scale extractions, or Ambion Rnaqueous-96 purification kit for 96-well assays. For Taqman analysis, dual-labeled probes are synthesized with, for example, the reporter dyes FAM or VIC covalently linked at the 5′-end and the quencher dye TAMARA conjugated to the 3′-end. One-step RT-PCR amplifications are performed on, for example, an ABI PRISM 7700 Sequence detector using 50 μL reactions consisting of 10 μL total RNA, 100 nM forward primer, 100 mM reverse primer, 100 nM probe, 1× TaqMan PCR reaction buffer (PE-Applied Biosystems), 5.5 mM MgCl2, 100 uM each dATP, dCTP, dGTP and dTTP, 0.2U RNase Inhibitor (Promega), 0.025 U AmpliTaq Gold (PE-Applied Biosystems) and 0.2 U M-MLV Reverse Transcriptase (Promega). The thermal cycling conditions can consist of 30 minutes at 48° C., 10 minutes at 95° C., followed by 40 cycles of 15 seconds at 95° C. and 1 minute at 60° C. Quantitation of target KRAS mRNA level is determined relative to standards generated from serially diluted total cellular RNA (300, 100, 30, 10 ng/rxn) and normalizing to, for example, 36B4 mRNA in either parallel or same tube TaqMan reactions.

Target gene expression levels (gene knockdown measurements by qRT-PCR) can be used to functionally confirm cellular entry, cytoplasmic delivery, and proper Dicing. This can also be supplemented with measuring the precise Ago2 cleavage point in the target RNA using 5′-RACE (e.g., Zhou et al., Nucleic Acids Res. 2009 May; 37(9):3094-109).

### Western Blotting

Nuclear extracts can be prepared using a standard micro preparation technique (see for example Andrews and Faller, 1991, Nucleic Acids Research, 19, 2499). Protein extracts from supernatants are prepared, for example using TCA precipitation. An equal volume of 20% TCA is added to the cell supernatant, incubated on ice for 1 hour and pelleted by centrifugation for 5 minutes. Pellets are washed in acetone, dried and resuspended in water. Cellular protein extracts are run on a 10% Bis-Tris NuPage (nuclear extracts) or 4-12% Tris-Glycine (supernatant extracts) polyacrylamide gel and transferred onto nitro-cellulose membranes. Non-specific binding can be blocked by incubation, for example, with 5% non-fat milk for 1 hour followed by primary antibody for 16 hours at 4° C. Following washes, the secondary antibody is applied, for example (1:10,000 dilution) for 1 hour at room temperature and the signal detected with SuperSignal remolecule (Pierce).

In several cell culture systems, cationic lipids have been shown to enhance the bioavailability of oligonucleotides to cells in culture (Bennet, et al., 1992, Mol. Pharmacology, 41, 1023-1033). In one embodiment, Dicer substrate molecules of the invention are complexed with cationic lipids for cell culture experiments. Dicer substrate and cationic lipid mixtures are prepared in serum-free DMEM immediately prior to addition to the cells. DMEM plus additives are warmed to room temperature (about 20-25° C.) and cationic lipid is added to the final desired concentration and the solution is vortexed briefly. DsiRNA molecules are added to the final desired concentration and the solution is again vortexed briefly and incubated for 10 minutes at room temperature. In dose response experiments, the RNA/lipid complex is serially diluted into DMEM following the 10 minute incubation.

### Animal Models

Evaluating the efficacy of dsRNA-peptide molecules in animal models is an important prerequisite to human clinical trials. Various animal models of cancer and/or proliferative diseases, conditions, or disorders as are known in the art can be adapted for use for pre-clinical evaluation of the efficacy of Dicer substrate compositions of the invention in modulating target gene expression toward therapeutic use.

For example, if the target is KRAS, as in cell culture models, the most Ras sensitive mouse tumor xenografts are those derived from cancer cells that express mutant Ras proteins. Nude mice bearing H-Ras transformed bladder cancer cell xenografts were sensitive to an anti-Ras antisense nucleic acid, resulting in an 80% inhibition of tumor growth after a 31 day treatment period (Wickstrom, 2001, Mol. Biotechnol., 18, 35-35). Zhang et al., 2000, Gene Ther., 7, 2041, describes an anti-KRAS ribozyme adenoviral vector (KRbz-ADV) targeting a KRAS mutant (KRAS codon 12 GGT to GTT; H441 and H1725 cells respectively). Non-small cell lung cancer cells (NSCLC H441 and H1725 cells) that express the mutant KRas protein were used in nude mouse xenografts compared to NSCLC H1650 cells that lack the relevant mutation. Pre-treatment with KRbz-ADV completely abrogated engraftment of both H441 and H1725 cells and compared to 100% engraftment and tumor growth in animals that received untreated tumor cells or a control vector. Additional mouse models of KRAS misregulation/mutation have also been described (e.g., in Kim et al. Cell 121: 823-835, which identified a role of KRAS in promoting lung adenocarcinomas). The above studies provide proof that inhibition of Ras expression (e.g., KRAS expression) by anti-Ras molecules causes inhibition of tumor growth in animals.

As such, these models can be used in evaluating the efficacy of Dicer substrate molecules of the invention in inhibiting KRAS levels, expression, tumor/cancer formation, growth, spread, development of other KRAS-associated phenotypes, diseases or disorders, etc. These models and others can similarly be used to evaluate the safety/toxicity and efficacy of Dicer substrate molecules of the invention in a pre-clinical setting.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

The present invention is described by reference to the following Examples, which are offered by way of illustration and are not intended to limit the invention in any manner. Standard techniques well known in the art or the techniques specifically described below were utilized.

### Example 1

### Preparation of a Dicer Substrate Aptamer

### Oligonucleotide Synthesis

Individual RNA strands are synthesized and HPLC purified according to standard methods (Integrated DNA Technologies, Coralville, Iowa). All oligonucleotides are quality control released on the basis of chemical purity by HPLC analysis and full length strand purity by mass spectrometry analysis. Dicer substrate aptamers formed by two polynucleotide strands are prepared before use by mixing equal quantities of each strand, briefly heating to 100° C. in RNA buffer (IDT) and then allowing the mixtures to cool to room temperature.

### Example 2

### Use of a Dicer Substrate Aptamer to Reduce Expression of a Target Gene in a Cell

Cell culture and RNA transfection

HeLa, Hep3B, HepG2, HT29, LS174T, and Neuro2a are obtained from ATCC and maintained in the recommended basal medium with 10% heat-inactivated FBS at 37° C. under 5% CO₂. For dsRNA and dsRNA-targeting peptide conjugate transfections, cells are transfected with the unconjugated or conjugated Dicer substrates as indicated at a final concentration of 1 nM or 0.1 nM. Lipofectamine™ RNAiMAX (Invitrogen). DsiRNAs are used as positive controls. Briefly, 2.5 μL of a 0.2 μM or 0.02 μM stock solution of each DsiRNAs is mixed with 47.5 μL of Opti-MEM I (Invitrogen). For Lipofectamine™ control, 2.5 μL of a 0.2 μM or 0.02 μM stock solution of each DsiRNAs is mixed with 46.5 μL of Opti-MEM I (Invitrogen) and 1 μL, of Lipofectamine™ RNAiMAX. The resulting 50 μL mix is added into individual wells of 12 well plates and incubated for 20 min at RT to allow DsiRNA:Lipofectamine™ RNAiMAX complexes to form. Meanwhile, cells are trypsinized and resuspended in medium at a final concentration of about 367 cells/μL. Finally, 450 μL of the cell suspension are added to each well (final volume 500 μL) and plates are placed into the incubator for 24 hours. For dose response study, the concentrations of Dicer substrates are varied from initially 1 pM to 1 nM. For time course studies, incubation times of about 4 hours to about 72 hours are studied.

### RNA Isolation and Analysis

Cells are washed once with 2 mL of PBS, and total RNA is extracted using RNeasy Mini Kit™ (Qiagen) and eluted in a final volume of 30 μL. 1 ng of total RNA is reverse-transcribed using Transcriptor 1^{st} Strand cDNA Kit™ (Roche) and randomized hexamers following manufacturer's instructions. One-thirtieth (0.66 μL) of the resulting cDNA is mixed with 5 μL of IQ Multiplex Powermix (Bio-Rad) together with 3.33 μL of H₂O and 1 μL of a 3 μM mix containing 2 sets of primers and probes specific for human genes HPRT-1 (accession number NM_{—}000194) KRAS and SFRS9 (accession number NM_{—}003769) genes:

Hu HPRT forward primer
F517 GACTTTGCTTTCCTTGGTCAG
Hu HPRT reverse primer
R591 GGCTTATATCCAACACTTCGTGGG
Hu HPRT probe
P554 Cy5-ATGGTCAAGGTCGCAAGCTTGCTGGT-IBFQ
Hu SFRS9 forward primer
F569 TGTGCAGAAGGATGGAGT
Hu SFRS9 reverse primer
R712 CTGGTGCTTCTCTCAGGATA
Hu SFRS9 probe
P644 HEX-TGGAATATGCCCTGCGTAAACTGGA-IBFQ

### Quantitative RT-PCR

A CFX96 Real-time System with a C1000 Thermal cycler (Bio-Rad) is used for the amplification reactions. PCR conditions are: 95° C. for 3 min; and then cycling at 95° C., 10 sec; 55° C., 1 min for 40 cycles. Each sample is tested in triplicate. Relative HPRT mRNA levels are normalized to SFRS9 mRNA levels and compared with mRNA levels obtained in control samples treated with the transfection remolecule plus a control mismatch duplex, or untreated. Data is analyzed using Bio-Rad CFX Manager version 1.0 software. Expression data are presented as a comparison of the expression under the treatment of dsRNA alone versus that of a Dicer substrate aptamer.

Dicer substrate aptamers are examined for efficacy of sequence-specific target mRNA inhibition. Specifically, HPRT-targeting Dicer substrate duplexes, HPRT-targeting Dicer substrate duplexes possessing a receptor binding region, and nucleic acid molecules with the receptor binding region and without the HPRT-targeting

Dicer substrate duplexes (if applicable) are transfected into HeLa cells at a fixed concentration of 20 nM and HPRT expression levels are measured 24 hours later. Transfections are performed in duplicate, and each duplicate is assayed in triplicate for HPRT expression by qPCR. Under these conditions (20 nM duplexes, Oligofectamine transfection), HPRT gene expression are reduced by at least 10%, 20%, 30%, 40%, 50%, 60%, 70%, 80%, 90% or more by HPRT-targeting Dicer substrate duplexes and HPRT-targeting Dicer substrate duplexes possessing aptamers, but not aptamers without the HPRT-targeting Dicer substrate duplexes. It is expected that HPRT-targeting Dicer substrate duplexes possessing aptamers have at least or about the same reduction in HPRT gene expression as HPRT-targeting Dicer substrate duplexes. However, the aptamers of HPRT-targeting Dicer substrate duplexes possessing aptamers may confer additional potency or efficacy over HPRT-targeting Dicer substrate duplexes alone. Thus it is shown that HPRT-targeting Dicer substrate duplexes possessing aptamers are effective at reducing target gene expression, and reducing target gene expression is dependent on the Dicer substrate portion of the HPRT-targeting Dicer substrate duplexes possessing aptamers.

### Example 3

### In Vitro Assay to Assess Serum Stability

Serum stability of Dicer substrate aptamers of the invention is assessed via incubation of Dicer substrate aptamer molecules in 50% fetal bovine serum for various periods of time (up to 24 h) at 37° C. Serum is extracted and the nucleic acids are separated on a 20% non-denaturing PAGE and visualized with Gelstar stain. Relative levels of protection from nuclease degradation are assessed for the Dicer substrate aptamers (optionally with and without modifications).

Thus, it can be shown that the Dicer substrate aptamers have increased serum stability and/or reduced degradation in serum. It is expected that the Dicer substrate aptamers have increased serum stability and/or reduced degradation in serum compared to a reference dsRNA (e.g., a Dicer substrate not joined to an aptamer). It can also be shown that the Dicer substrate aptamers of the invention reduce gene expression of a specific target, esp. in comparison to a reference dsRNA.

### Example 4

### In Vivo Efficacy of Dicer Substrate Aptamers

To demonstrate the capability of the Dicer substrate aptamers of the invention to reduce gene expression of specific target genes in vivo, such molecules are administered to mice, either systemically (e.g., by hydrodynamic injection) or via direct injection of a tissue (e.g., injection of the eye, spinal cord/brain/CNS, etc.). Measurement of target RNA levels are performed upon target cells (e.g., RNA levels in liver and/or kidney cells are assayed following hydrodynamic tail-vein injection of mice; eye cells are assayed following ophthalmic injection of subjects; or spinal cord/brain/CNS cells are assayed following direct injection of same of subjects) by standard methods (e.g., Trizol® preparation (guanidinium thiocyanate-phenol-chloroform) followed by qRT-PCR).

Exemplary liver target RNAs include Hypoxanthine-Guanine Phosphoribosyl Transferase (HPRT1; GenBank Accession No. NM_{—}013556); Glyceraldehyde 3-Phosphate Dehydrogenase (GAPDH; GenBank Accession No. NM_{—}008084); Lamin A (LMNA; GenBank Accession No. NM_{—}019390); Heterogeneous Nuclear Ribonucleoprotein A1 (HNRPA1; GenBank Accession No. NM_{—}010447) and ATPase, Na+/K+ Transporting, Beta 3 Polypeptide (ATP1B3; GenBank Accession No. NM_{—}007502). Such target genes are selected from among art-recognized “housekeeping” genes, with housekeeping genes selected as target genes for the double purposes of assuring that target genes possess strong and homogenous expression in mouse liver tissues and of minimizing inter-animal expression level variability. In one set of experiments, mice weighing approximately 25 grams (e.g., CD-1, C57BL/6, A/J or other commercially available strain of mouse) are purchased, housed, treated and sacrificed (with such handling performed in accordance with Institutional Review Board policies). Dicer substrate aptamers are synthesized to target the above-recited liver target RNAs, with two distinct sites targeted within the ATP1B3 transcript. 200 mg doses of Dicer substrate aptamers of the invention are dissolved in phosphate-buffered saline (PBS; 2.5 mL total volume per dose) and are administered to mice as single hydrodynamic injections through the tail vein. Liver samples are then collected from dosed mice at 24 hours after administration. A total of five to ten animals per group are treated with each Dicer substrate aptamer molecule. Target mRNA levels are assessed using quantitative reverse transcriptase-polymerase chain reaction (“qRT-PCR”). cDNAs are synthesized using a mix of oligo-dT and randomized hexamer priming. qPCR reactions are run in triplicate. Absolute quantification is performed by extrapolation against a standard curve run against a cloned linearized amplicon target. Data are normalized, setting the control gene expression level to be the measured target mRNA expression values for all mice not administered target mRNA-specific Dicer substrate aptamers molecules, which are averaged to obtain a 100% control value (e.g., for mice injected with GAPDH targeting Dicer substrate aptamers, the set of HPRT1, LMNA, HNRPA1, ATP1B3-1 and ATP1B3-3 mice are all used as negative controls to yield normalized, basal GAPDH levels. Thus, there are five to ten study mice and 25-50 control mice for each arm of the study). Normalized qRT-PCR results are then assessed to determine Dicer substrate aptamers possessing statistically significant reduction of target RNA levels (RNA interference efficacy).

In any of the above-described in vivo experiments, a Dicer substrate aptamers (e.g., single stranded or double stranded Dicer substrate aptamer) of the invention containing a Dicer substrate molecule can be deemed to be an effective in vivo molecule if a statistically significant reduction in RNA levels is observed when adminstering a Dicer substrate aptamer of the invention, as compared to an appropriate control (e.g., a vehicle alone control, a randomized duplex control, a duplex directed to a different target RNA control, an aptamer control, a dsRNA directed to the same target RNA control etc.). Generally, if the p-value (e.g., generated via 1 tailed, unpaired T-test) assigned to such comparison is less than 0.05, a Dicer substrate aptamer of the invention is deemed to be an effective RNA interference molecule. Alternatively, the p-value threshold below which to classify a Dicer substrate aptamer of the invention as an effective RNA interference molecule can be set, e.g. at 0.01, 0.001, etc., in order to provide more stringent filtering, identify more robust differences, and/or adjust for multiple hypothesis testing, etc. Absolute activity level limits can also be set to distinguish between effective and non-effective Dicer substrate aptamers. For example, in certain embodiments, an effective Dicer substrate aptamer of the invention is one that not only shows a statistically significant reduction of target RNA levels in vivo but also exerts, e.g., at least an approximately 10% reduction, approximately 15% reduction, at least approximately 20% reduction, approximately 25% reduction, approximately 30% reduction, etc. in target RNA levels in the tissue or cell that is examined, as compared to an appropriate control. The in vivo efficacy of the Dicer substrate aptamer of the invention is thereby demonstrated.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications can be made to the invention disclosed herein without departing from the scope and spirit of the invention. Thus, such additional embodiments are within the scope of the present invention and the following claims. The present invention teaches one skilled in the art to test various combinations and/or substitutions of chemical modifications described herein toward generating nucleic acid constructs with improved activity for mediating RNAi activity. Such improved activity can comprise improved stability, improved bioavailability, and/or improved activation of cellular responses mediating RNAi. Therefore, the specific embodiments described herein are not limiting and one skilled in the art can readily appreciate that specific combinations of the modifications described herein can be tested without undue experimentation toward identifying Dicer substrate aptamers.

The invention illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. An isolated nucleic acid molecule comprising:
a polynucleotide strand having a 5′ terminus and a 3′ terminus that is 53-142 nucleotides in length, said 5′ terminus and said 3′ terminus forming a double-stranded region of at least 21-25 base pairs, wherein said double-stranded region comprises at least 19 nucleotides complementary to a target RNA,
wherein said nucleic acid molecule selectively binds a receptor with an affinity of at least 100 μM,
wherein Dicer cleavage of said nucleic acid molecule reduces target gene expression in a mammalian cell, and reduces the ability of said isolated nucleic acid to bind selectively to the receptor.

2. An isolated nucleic acid molecule comprising:
a first polynucleotide strand having a 5′ terminus and a 3′ terminus that is 33-121 nucleotides in length and a second polynucleotide strand having a 5′ terminus and a 3′ terminus that is 33-121 nucleotides in length, said 5′ terminus of said first polynucleotide strand and said 3′ terminus of said second polynucleotide strand forming a double-stranded region of at least 21-25 base pairs, wherein said double-stranded region comprises at least 19 nucleotides complementary to a target RNA,
wherein said nucleic acid molecule selectively binds a receptor with an affinity of at least 100 μM,
wherein Dicer cleavage of said nucleic acid molecule target gene expression in a mammalian cell, and reduces the ability of said isolated nucleic acid to bind selectively to the receptor.

3. The isolated nucleic acid molecule of claim 1, wherein said 5′ terminus of said molecule comprises a terminal 5′ nucleotide and a penultimate 5′ nucleotide and said 3′ terminus of said molecule comprises a 3′ nucleotide and a penultimate 3′ nucleotide, wherein the 5′ nucleotide and 5′ penultimate nucleotide of said 5′ terminus and the 3′ nucleotide and 3′ penultimate nucleotide of said 3′ terminus correspond in a duplex so as to form a complementary base paired blunt end.

4. The isolated nucleic acid molecule of claim 1, wherein said 5′ terminus of said molecule comprises a terminal 5′ nucleotide and said 3′ terminus of said molecule comprises a 3′ nucleotide (position 3′-1), a penultimate 3′ nucleotide (position 3′-2), and two successive consecutive 3′ internal nucleotides (positions 3′-3 and 3′-4), wherein the 5′ nucleotide of said 5′ terminus is paired with its corresponding nucleotide of said 3′ terminus, and wherein 1-4 nucleotides of the 3′ terminus form a 3′ single stranded overhang.

5. The isolated nucleic acid molecule of claim 1, wherein said 5′ terminus of said molecule comprises a terminal 5′ nucleotide and a penultimate 5′ nucleotide and said 3′ terminus of said molecule comprises a 3′ nucleotide and a penultimate 3′ nucleotide, wherein the 5′ nucleotide and 5′ penultimate nucleotide of said 5′ terminus and the 3′ nucleotide and 3′ penultimate nucleotide of said 3′ terminus correspond in a duplex so as to form one or two mismatched base pairs.

6. The isolated nucleic acid molecule of claim 2, wherein the said 5′ terminus of said molecule comprises a terminal 5′ nucleotide and a penultimate 5′ nucleotide and said 3′ terminus of said molecule comprises a 3′ nucleotide and a penultimate 3′ nucleotide, wherein the 5′ nucleotide and 5′ penultimate nucleotide of said 5′ terminus and the 3′ nucleotide and 3′ penultimate nucleotide of said 3′ terminus correspond in a duplex so as to form a complementary base paired blunt end.

7. The isolated nucleic acid molecule of claim 2, wherein said 5′ terminus of first nucleotide strand of said molecule comprises a terminal 5′ nucleotide and said 3′ terminus of said second nucleotide strand of said molecule comprises a 3′ nucleotide (position 3′-1), a penultimate 3′ nucleotide (position 3′-2), and two successive consecutive 3′ internal nucleotides (positions 3′-3 and 3′-4), wherein the 5′ nucleotide of said 5′ terminus is paired with its corresponding nucleotide of said 3′ terminus, and wherein 1-4 nucleotides of the 3′ terminus form a 3′ single stranded overhang.

8. The isolated nucleic acid molecule of claim 2, wherein said 5′ terminus of first polynucleotide strand of said molecule comprises a terminal 5′ nucleotide and a penultimate 5′ nucleotide and said 3′ terminus of said second polynucleotide strand of said molecule comprises a 3′ nucleotide and a penultimate 3′ nucleotide, wherein the 5′ nucleotide and 5′ penultimate nucleotide of said 5′ terminus and the 3′ nucleotide and 3′ penultimate nucleotide of said 3′ terminus correspond in a duplex so as to form one or two mismatched base pairs.

9. The isolated nucleic acid molecule of claim 1, wherein said receptor binding affinity is 1-100 μM.

10. The isolated nucleic acid molecule of claim 1, wherein said receptor binding affinity is 1-100 nm.

11. The isolated nucleic acid molecule of claim 1, wherein said receptor binding affinity is 1-100 pm.

12. The isolated nucleic acid molecule of claim 1, wherein the isolated nucleic acid forms a hairpin comprising an internally base-paired region and a single-stranded region, said internally base-paired region comprising at least 4 consecutive base pairs and said single-stranded region comprising at least 5 consecutive non-base paired nucleotides, wherein said receptor binding affinity is dependent upon the presence of said hairpin in said isolated nucleic acid.

13. The isolated nucleic acid molecule of claim 1, wherein said receptor is expressed on the surface of a cell.

14. The isolated nucleic acid molecule of claim 13, wherein the receptor is selected from the group consisting of: nucleolin, a human epidermal growth factor receptor 2 (HER2), CD20, a transferrin receptor, an asialoglycoprotein receptor, a thyroid-stimulating hormone (TSH) receptor, a fibroblast growth factor (FGF) receptor, CD3, the interleukin 2 (IL-2) receptor, a growth hormone receptor, an insulin receptor, an acetylcholine receptor, an adrenergic receptor, a vascular endothelial growth factor (VEGF) receptor, a protein channel, cadherin, a desmosome, and a viral receptor.

15. The isolated nucleic acid molecule of claim 1, wherein said receptor is internalized into a mammalian cell by an amount (expressed by %) selected from the group consisting of: at least 10%, at least 50% and at least 80-90%.

16. The isolated nucleic acid molecule of claim 1, wherein the isolated nucleic acid molecule is cleaved in a mammalian cell to produce a double-stranded ribonucleic acid (dsRNA) of 19-23 nucleotides in length that reduces target gene expression.

17. The isolated nucleic acid molecule of claim 1, wherein the isolated nucleic acid molecule reduces target gene expression in a mammalian cell in vitro by an amount (expressed by %) selected from the group consisting of: at least 10%, at least 50% and at least 80-90%.

18. The isolated nucleic acid molecule of claim 1, wherein the isolated nucleic acid molecule, when introduced into a mammalian cell, reduces target gene expression in comparison to a reference dsRNA.

19. The isolated nucleic acid molecule of claim 1, wherein the isolated nucleic acid molecule, when introduced into a mammalian cell, reduces target gene expression by at least 70% relative to a negative control when transfected into said cell at a concentration selected from the group consisting of: 1 nM or less, 200 pM or less, 100 pM or less, 50 pM or less, 20 pM or less and 10 pM or less.

20. The isolated nucleic acid molecule of claim 1, wherein Dicer cleavage results in unfolding of said isolated nucleic acid molecule by an amount (expressed by %) selected from the group consisting of: at least 10%, at least 50% and at least 80-90%.

21. The isolated nucleic acid molecule of claim 1, wherein Dicer cleavage decreases the stability of the isolated nucleic acid molecule by an amount (expressed by %) selected from the group consisting of: at least 10%, at least 50% and at least 80-90%.

22. The isolated nucleic acid molecule of claim 1, wherein Dicer cleavage increases the degradation of the isolated nucleic acid molecule by an amount (expressed by %) selected from the group consisting of: at least 10%, at least 50% and at least 80-90%.

23. The isolated nucleic acid of claim 1, comprising a modified nucleotide.

24. The isolated nucleic acid of claim 23, wherein said modified nucleotide 10 residue is selected from the group consisting of: 2′-O-methyl, 2′-methoxyethoxy, 2′-fluoro, 2′-allyl, 2′-O-[2-(methylamino)-2-oxoethyl], 4′-thio, 4′-CH2-O-2′-bridge, 4′(CH2)2-O-2′-bridge, 2′-LNA, 2′-amino and 2′-O-(N-methlycarbamate).

25. The isolated nucleic acid molecule of claim 23, wherein the isolated nucleic acid molecule has increased nuclease resistance relative to a reference dsRNA.

26. The isolated nucleic acid molecule of claim 23, wherein Dicer cleavage decreases the nuclease resistance of the isolated nucleic acid molecule by an amount (expressed by %) selected from the group consisting of: at least 10%, at least 50% and at least 80-90%.

27. The isolated nucleic acid of claim 4, wherein said nucleotides of said 3′ single stranded overhang comprise a modified nucleotide.

28. The isolated nucleic acid of claim 27, wherein said 3′ overhang is two nucleotides in length and wherein said modified nucleotide of said 3′ overhang is a 2′-O-methyl modified ribonucleotide.

29. The isolated nucleic acid molecule of claim 1, wherein the isolated nucleic acid molecule is susceptible to Dicer cleavage, as determined by an in vitro dicer cleavage assay in which at least 10% of the amount of said molecule introduced into the assay is cleaved to produce a 21-23 bp double stranded nucleic acid molecule.

30. The isolated nucleic acid molecule of claim 1, wherein the isolated nucleic acid molecule is identified using systematic evolution of ligands by exponential enrichment (SELEX).

31. A method of making a nucleic acid molecule that selectively binds a receptor and is a Dicer substrate, comprising:
providing a nucleic acid molecule comprising a single polynucleotide strand having a 5′ terminus and a 3′ terminus that is 53-142 nucleotides in length, said 5′ terminus and said 3′ terminus forming a double-stranded region of at least 21-25 base pairs, wherein said double-stranded region comprises at least 19 nucleotides complementary to a target RNA;
contacting the nucleic acid molecule with a receptor;
isolating the nucleic acid molecule bound to the receptor; and
contacting the isolated nucleic acid molecule with Dicer enzyme, wherein Dicer cleavage of the nucleic acid molecule reduces the ability of the isolated nucleic acid molecule to bind selectively to the receptor, thereby making a nucleic acid molecule that selectively binds a receptor and is a substrate for Dicer cleavage.

32. A method of making a nucleic acid molecule that selectively binds a receptor and is a Dicer substrate, comprising:
providing a nucleic acid molecule comprising a first polynucleotide strand having a 5′ terminus and a 3′ terminus that is 33-121 nucleotides in length and a second polynucleotide strand having a 5′ terminus and a 3′ terminus that is 33-121 nucleotides in length, said 5′ terminus of said first polynucleotide strand and said 3′ terminus of said second polynucleotide strand forming a double-stranded region of at least 21-25 base pairs, wherein said double-stranded region comprises at least 19 nucleotides complementary to a target RNA;
contacting the nucleic acid molecule with a receptor;
isolating the nucleic acid molecule bound to the receptor; and
contacting the isolated nucleic acid molecule with Dicer enzyme, wherein Dicer cleavage of the nucleic acid molecule in said double-stranded region reduces the ability of the isolated nucleic acid molecule to bind selectively to the receptor, thereby making a nucleic acid molecule that selectively binds a receptor and is a Dicer substrate.

33. A method of making a nucleic acid molecule that selectively binds a receptor and is a Dicer substrate, comprising:
providing a nucleic acid molecule comprising (a) an aptamer comprising a single polynucleotide strand having a 5′ terminus and a 3′ terminus that is 12-100 nucleotides in length, and (b) a double-stranded RNA (dsRNA) comprising a first strand that is 25-30 nucleotides in length and a second strand that is 25-34 nucleotides in length, wherein the 3′ terminus of said first strand is covalently attached to the 5′ terminus of said aptamer and the 5′ end of said second strand is covalently attached to the 3′ terminus of said aptamer;
contacting the nucleic acid molecule with a receptor;
isolating the nucleic acid molecule bound to the receptor; and
contacting the isolated nucleic acid molecule with Dicer enzyme, wherein Dicer cleavage of said dsRNA reduces the ability of the aptamer to bind selectively to the receptor, thereby making a nucleic acid molecule that selectively binds a receptor and is a Dicer substrate.

34. A method of making a nucleic acid molecule that selectively binds a receptor and is a Dicer substrate, comprising:
providing a nucleic acid molecule comprising (a) an aptamer comprising a first polynucleotide strand having a 5′ terminus and (b) a 3′ terminus that is 12-100 nucleotides in length and a second polynucleotide strand having a 5′ terminus and a 3′ terminus that is 12-100 nucleotides in length, and a double-stranded RNA (dsRNA) comprising a first strand that is 25-30 nucleotides in length and a second strand that is 25-34 nucleotides in length, wherein the 3′ terminus of the first strand of said dsRNA is covalently attached to the 5′ terminus of the first strand of said aptamer and the 5′ end of said second strand of said dsRNA is covalently attached to the 3′ terminus of said aptamer;
contacting the nucleic acid molecule with a receptor;
isolating the nucleic acid molecule bound to the receptor; and
contacting the nucleic acid molecule with Dicer enzyme, wherein Dicer cleavage of said dsRNA reduces the ability of the aptamer to bind selectively to the receptor, thereby making a nucleic acid molecule that selectively binds a receptor and is a Dicer substrate.

35. The method of claim 31, wherein said 5′ terminus and said 3′ terminus form a blunt end.

36. The method of claim 31, wherein said 5′ terminus and said 3′ terminus form a 1-4 nucleotide 3′ overhang.

37. The method of claim 31, wherein the first two nucleotides of said 5′ terminus and the ultimate and penultimate nucleotides of said 3′ terminus form one or two mismatched base pairs.

38. The method of claim 32, wherein the 5′ terminus of said first polynucleotide strand and the 3′ terminus of said second polynucleotide strand form a blunt end.

39. The method of claim 32, wherein the 5′ terminus of said first polynucleotide strand and the 3′ terminus of said second polynucleotide strand form a 1-4 nucleotide 3′ overhang.

40. The method of claim 31, wherein the 5′ terminus of said first polynucleotide strand and the 3′ terminus of said second polynucleotide strand form one or two mismatched base pairs.

41. The method of claim 32, further comprising contacting the isolated nucleic acid molecule Dicer cleaved nucleic acid molecule with the receptor and determining binding to the receptor.

42. The method of claim 32, wherein the method comprises systematic evolution of ligands by exponential enrichment (SELEX).

43. The method of claim 32, wherein said receptor binding affinity is 1-100 μM.

44. The method of claim 32, wherein said receptor binding affinity is 1-100 nm.

45. The method of claim 32, wherein said receptor binding affinity is 1-100 pm.

46. The method of claim 32, wherein the isolated nucleic acid contains an internally base-paired region and a single-stranded region forming a hairpin, said internally base-paired region comprising 4 consecutive base pairs and said single-stranded region comprising 5 consecutive non-base paired nucleotides, wherein said receptor binding affinity is dependent upon the presence of said hairpin in said isolated nucleic acid.

47. The method of claim 32, wherein said receptor is expressed on the surface of a cell.

48. The method of claim 47, wherein the receptor is selected from the list consisting of nucleolin, a human epidermal growth factor receptor 2 (HER2), CD20, a transferrin receptor, an asialoglycoprotein receptor, a thyroid-stimulating hormone (TSH) receptor, a fibroblast growth factor (FGF) receptor, CD3, the interleukin 2 (IL-2) receptor, a growth hormone receptor, an insulin receptor, an acetylcholine receptor, an adrenergic receptor, a vascular endothelial growth factor (VEGF) receptor, a protein channel, cadherin, a desmosome, and a viral receptor.

49. The method of claim 32, wherein said receptor is internalized into a mammalian cell by an amount (expressed by %) selected from the group consisting of: at least 10%, at least 50% and at least 80-90%.

50. The method of claim 32, wherein the isolated nucleic acid molecule is cleaved endogenously in a mammalian cell to produce a double-stranded ribonucleic acid (dsRNA) of 19-23 nucleotides in length that reduces target gene expression.

51. The method of claim 32, wherein the isolated nucleic acid molecule reduces target gene expression in a mammalian cell in vitro by an amount (expressed by %) selected from the group consisting of: at least 10%, at least 50% and at least 80-90%.

52. The method of claim 32, wherein the isolated nucleic acid molecule, when introduced into a mammalian cell, reduces target gene expression in comparison to a reference dsRNA.

53. The method of claim 32, wherein the isolated nucleic acid molecule, when introduced into a mammalian cell, reduces target gene expression by at least 70% when transfected into said cell at a concentration selected from the group consisting of: 1 nM or less, 200 pM or less, 100 pM or less, 50 pM or less, 20 pM or less and 10 pM or less.

54. The method of claim 32, wherein Dicer cleavage results in unfolding of said isolated nucleic acid molecule by an amount (expressed by %) selected from the group consisting of: at least 10%, at least 50% and at least 80-90%.

55. The method of claim 32, wherein Dicer cleavage decreases the stability of the isolated nucleic acid molecule by an amount (expressed by %) selected from the group consisting of: at least 10%, at least 50% and at least 80-90%.

56. The method of claim 32, wherein Dicer cleavage increases the degradation of the isolated nucleic acid molecule by an amount (expressed by %) selected from the group consisting of: at least 10%, at least 50% and at least 80-90%.

57. The method of claim 1, wherein the nucleic acid molecule comprises a modified nucleotide.

58. The method of claim 57, wherein said modified nucleotide residue is selected from the group consisting of: 2′-O-methyl, 2′-methoxyethoxy, 2′-fluoro, 2′-allyl, 2′-O-[2-(methyl amino)-2-oxoethyl], 4′-thio, 4′-CH2-O-2′-bridge, 4′-(CH2)2-O-2′-bridge, 2′-LNA, 2′-amino and 2′-O-(N-methlycarbamate).

59. The method of claim 57, wherein the nucleic acid molecule has increased nuclease resistance relative to a reference dsRNA.

60. The method of claim 57, wherein Dicer cleavage decreases the nuclease resistance of the nucleic acid molecule by an amount (expressed by %) selected from the group consisting of: at least 10%, at least 50% and at least 80-90%.

61. The method of claim 36, wherein said nucleotides of said 3′ overhang comprise a modified nucleotide.

62. The method of claim 61, wherein said 3′ overhang is two nucleotides in length and wherein said modified nucleotide of said 3′ overhang is a 2′-O-methyl modified ribonucleotide.

63. The method of claim 32, wherein the isolated nucleic acid molecule is susceptible to Dicer cleavage, as determined by an in vitro dicer cleavage assay in which at least 10% of the amount of said molecule introduced into the assay is cleaved to produce a 21-23 bp double stranded nucleic acid molecule.

64. An isolated nucleic acid molecule made by the method of claim 32.
